(19) United States
(12) Patent Application Publication
(10) Pub. No.: US 2023/0265083 A1
(43) Pub. Date: Aug. 24, 2023

(54) Heterocycle CDK Inhibitors And Their Use Thereof

(71) Applicant: Prelude Therapeutics Incorporated, Wilmington, DE (US)

(72) Inventors: Liang Lu, Hockessin, DE (US); Andrew P. Combs, Kennett Square, PA (US); Chaofeng Dai, Kennett Square, PA (US); Raul A. Leal, Newark, DE (US)

(21) Appl. No.: 18/041,223

(22) PCT Filed: Aug. 10, 2021
(86) PCT No.: PCT/US2021/045312

§ 371 Date: Feb. 10, 2023

Related U.S. Application Data
(60) Provisional application No. 63/063,569, filed on Aug. 10, 2020.

(51) Int. Cl. C07D 413/04; C07D 401/04; C07D 405/04

(52) U.S. Cl. CPC C07D 413/04; C07D 401/04; C07D 405/04

(57) ABSTRACT

The disclosure is directed to, in part, to heterocycle CDK inhibitors, pharmaceutical compositions comprising the same, as well as methods of their use and preparation.

### TECHNICAL FIELD

The disclosure is directed to heterocycle CDK inhibitors and methods of their use.

### BACKGROUND

Cyclin-dependent kinases (CDK), a family of serine/threonine kinases whose activities are dependent on association and activation by cyclins, play critical roles in regulating cell cycle and gene transcription (Malumbres, M. (2014). “Cyclin-dependent kinases.” Genome Biol 15(6): 122.). While CDK1, CDK2, CDK4, and CDK6 are directly involved in promoting cell division, other members such as CDK7, CDK8, and CDK9 comprise a second subgroup that regulates transcription.

Upon gene activation, transcription activators, co-activators/mediator complex (MC), and RNA polymerase II (RNA Pol II) first assemble on the gene promoter to form the pre-initiation complex (PIC). RNA Pol II is then released from the complex upon PIC activation to start initial transcription, only to be paused by factors like negative elongation factors (NELF) and DRB sensitivity induced factors (DSIF) shortly after. This is termed promoter pausing. Productive elongation does not occur until paused RNA Pol II is released from pausing by positive transcription elongation factor b (p-TEFb) (Harlen, K. M. and L. S. Churchman (2017). “The code and beyond: transcription regulation by the RNA polymerase II carboxy-terminal domain.” Nat Rev Mol Cell Biol 18(4): 263-273.). During this process, CDK8, as a subunit of MC, facilitates PIC formation, while CDK7, a component of transcription factor IIH (TFIIH), phosphorylates Serine-5/7 of RNA Pol II C-terminal domain (CTD) to trigger its escape from the promoter region, and CDK9, the catalytic subunit of p-TEFb, phosphorylates Serine-2 of CTD as well as NEFL, DSIF to release RNA Pol II from pausing, allowing it to elicit productive elongation (Franco, L. C., et al. (2018). “CDK9: A key player in cancer and other diseases.” J Cell Biochem 119(2): 1273-1284; Soutourina, J. (2018). “Transcription regulation by the Mediator complex.” Nat Rev Mol Cell Biol 19(4): 262-274.)).

As the master regulator controlling releasing of paused Pol II from the promoter, CDK9 plays pivotal roles in promoting gene expression. Consistently, inhibition of CDK9 triggers global down-regulation of gene expression (Olson, C. M., et al. (2018). “Pharmacological perturbation of CDK9 using selective CDK9 inhibition or degradation.” Nat Chem Biol 14(2): 163-170.), among which are short-lived transcripts, such as the oncogene, c-Myc, and Mcl-1, a member of pro-survival Bcl-2 family of proteins that promote cancer cell survival (Chen, R., et al. (2005). “Transcription inhibition by flavopiridol: mechanism of chronic lymphocytic leukemia cell death.” Blood 106(7): 2513-2519; Youle, R. J. and A. Strasser (2008). “The BCL-2 protein family: opposing activities that mediate cell death.” Nat Rev Mol Cell Biol 9(1): 47-59.), suggesting an indirect approach to target Mcl-1 to treat cancer (Krystof, V., et al. (2012). “Perspective of cyclin-dependent kinase 9 (CDK9) as a drug target.” Curr Pharm Des 18(20): 2883-2890). Indeed, several CDK9 inhibitors have been developed and showed promising anti-cancer activities in preclinical models and have been advanced into the clinic (Boffo, S., et al. (2018). “CDK9 inhibitors in acute myeloid leukemia.” J Exp Clin Cancer Res 37(1): 36.). Interestingly, a recent study found that CDK9 inhibition also reactivates epigenetically silenced tumor suppressor genes, adding another line of evidence that supports targeting CDK9 for cancer therapy (Zhang, H., et al., (2018). “Targeting CDK9 Reactivates Epigenetically Silenced Genes in Cancer.” Cell 175(5): 1244-1258.e1226).

### SUMMARY

In some embodiments, compounds, or pharmaceutically acceptable salts thereof, are provided that, in part, modulate the activity of the CDK such as CDK9. The compounds can have, for example, a formula as described herein. In some embodiments, the compound is selected from a compound described herein. In some embodiments, methods of treating the conditions described herein are provided. In some embodiments, the condition is cancer and the like.

In some embodiments, the compound is a compound having a formula of

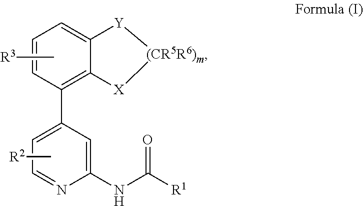

or a pharmaceutically acceptable salt thereof, wherein X, Y, m, R¹, R², R³, R⁵, and R⁶ are as provided for herein and, for example, can be selected from the respective groups of chemical moieties described herein. Also provided are processes for preparing these compounds.

In some embodiments, methods of inhibiting a CDK enzyme are provided, the method comprising: contacting the CDK enzyme with an effective amount of a compound as provided herein, or a pharmaceutically acceptable salt or solvate thereof, or a pharmaceutical composition comprising the same.

In some embodiments, methods of treating a disease or disorder associated with aberrant CDK activity in a subject or a subject in need thereof are provided, the method comprising administering to the subject, a compound as provided herein, or a pharmaceutically acceptable salt or solvate thereof, or a pharmaceutical composition comprising the same.

In some embodiments, methods of treating cancer in a subject or a subject in need thereof are provided, the method comprising administering to the subject, a compound as identified or provided herein, or a pharmaceutically acceptable salt or solvate a pharmaceutical composition comprising the same. In some embodiments, the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer

In some embodiments, pharmaceutical compositions comprising one or more compounds as identified or provided herein, or a pharmaceutically acceptable salt or solvate thereof, or a compound of the various formula provided herein, or a pharmaceutically acceptable salt or solvate thereof, are provided.

Stereoisomers of the compounds of the various formula provided herein, and pharmaceutical salts and solvates thereof, are also contemplated, described, and encompassed herein. Methods of using compounds of the formula provided herein are described, as well as pharmaceutical compositions including the compounds of the formula as identified or provided herein.

### DETAILED DESCRIPTION OF ILLUSTRATIVE EMBODIMENTS

The disclosure may be more fully appreciated by reference to the following description, including the following definitions and examples. Certain features of the disclosed compositions and methods that are described herein in the context of separate aspects may also be provided in combination in a single aspect. Alternatively, various features of the disclosed compositions and methods that are, for brevity, described in the context of a single aspect, may also be provided separately or in any subcombination.

At various places in the present specification, substituents of compounds are disclosed in groups or in ranges. It is specifically intended that the embodiments include each and every individual subcombination of the members of such groups and ranges. For example, the term “C₁₋₆ alkyl” or “C₁-C₆ alkyl” is specifically intended to individually disclose methyl, ethyl, C₃ alkyl, C₄ alkyl, C₅ alkyl, and C₆ alkyl.

It is further appreciated that certain embodiments, which are, for clarity, described in the context of separate embodiments, can also be provided in combination in a single embodiment. Conversely, various features of the embodiments, which are, for brevity, described in the context of a single embodiment, can also be provided separately or in any suitable sub-combination.

All percentages and ratios used herein, unless otherwise indicated, are by weight.

The term “alkyl,” when used alone or as part of a substituent group, refers to a straight- or branched-chain hydrocarbon group, a spirocyclic group, or a fused or bridged bicyclic group, each of which has from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. The term “spirocyclic group” refers to spirocyclic compounds in which the two rings share only one single atom, the spiro atom, which is usually a quaternary carbon. Examples of spirocyclic compounds are spiro[2,3]undecane, spiro[3,3]heptane, and spiro[5,5]undecane. The term “fused bicyclic group” refers to fused bicyclic compounds, in which two rings share two adjacent atoms. Examples of fused bicyclic compounds include bicyclo[4.4.0]decane, α-thujene, and decalin and the like. The term “bridged bicyclic group” refers to bridged bicyclic compounds, in which the two rings share three or more atoms, separating the two bridgehead atoms by a bridge containing at least one atom. Examples of bridged bicyclic compounds include bicyclo[2.2.1]heptane, bicyclo[1,1,1]pentane, bicyclo[2.2.1]heptane, bicyclo[2.2.2]octane, bicyclo-[3.3.1]nonane, bicyclo[3.3.3]undecane, and the like. The term “haloalkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group, wherein one or more of the hydrogen atoms in the group have been replaced by a halogen atom. Examples of haloalkyl groups include trifluoromethyl (—CF₃, C₁haloalkyl), trifluoroethyl (—CH₂CF₃, C₁haloalkyl), and the like.

The term “halo” or “halogen” refers to chloro, fluoro, bromo, or iodo.

The term “oxo” refers to an oxygen atom (i.e., ═O) as a divalent substituent, forming a carbonyl group when attached to a carbon (e.g., C═O), or attached to a nitrogen or sulfur heteroatom forming a nitroso, sulfinyl or sulfonyl.

The term “cycloalkyl” when used alone or as part of a substituent group refers to monocyclic, bicyclic, or tricyclic, non-aromatic hydrocarbon groups having from 3 to 10 carbon atoms (“C₃-C₁₀”), preferably from 3 to 6 carbon atoms (“C₃-C₆”), or from 3 to 7 carbon atoms (“C₃-C₇”). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like.

The term “heterocycloalkyl” when used alone or as part of a substituent group refers to any three to fourteen membered monocyclic, bicyclic, or tricyclic saturated ring structure containing at least one heteroatom selected from the group consisting of 0, N, and S. Heterocycloalkyl groups may be described with respect to the number of atoms in the group, or with respect to the number of carbon atoms in the group. The term “bicyclic” ring structure refers to a spirocyclic, fused bicyclic, or bridged bicyclic ring. For example, the term “4-10 membered heterocycloalkyl” refers to a heterocycloalkyl group containing between 4 and 10 ring atoms. The term —C₄-C₆ heterocycloalkyl, for example, refers to a heterocycloalkyl group containing four to six carbon atoms. The heterocycloalkyl group may be attached at any heteroatom or carbon atom of the ring such that the result is a stable structure. Examples of suitable heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, decahydroquinoline, 2-azaspiro[5.5]undecane, 6-oxa-3-azabicyclo[3.1.1]heptane, and the like.

The term “aryl” when used alone or as part of a substituent group refers to a mono- or bicyclic-aromatic hydrocarbon ring structure having 6 or 10 carbon atoms in the ring system. Examples of aryl groups are phenyl and naphthyl.

The term “heteroaryl” when used alone or as part of a substituent group refers to a mono-, bi-, or tricyclic-aromatic ring structure including carbon atoms as well as up to four heteroatoms selected from nitrogen, oxygen, and sulfur. Heteroaryl rings can include a total of 5, 6, 9, 10, or 14 ring atoms. Heteroaryl groups may be described with respect to the number of atoms in the group, or with respect to the number of carbon atoms in the group. Thus, the term “5-14 membered heteroaryl” refers to a heteroaryl group containing between 5 and 14 ring atoms. The term —C₄-C₆ heteroaryl, for example, refers to a heteroaryl group containing four to six carbon atoms. Examples of heteroaryl groups include but are not limited to pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. When a range of carbon atoms is used herein, for example, C₁-C₆, all ranges as well as individual numbers of carbon atoms are encompassed. For example, “C₁-C₃” includes C₁-C₃, C₁-C₂, C₂-C₃, C₁, C₂, and C₃. The range of carbon atoms may be expressed with alternative expressions. For example, the term “C₁₋₆” is an alternative expression of “C₁-C₆”. When a ring system is described herein as having a range of members, for example, “5-14-membered”, all ranges, as well as individual numbers of atoms, are encompassed. For example, “5-14-membered” includes 5-6-membered, 5-10-membered, 6-9-membered, 5-membered, 6-membered, 7-membered, 8-membered, and the like.

As used herein, “alkoxy” refers to an —O-alkyl group. Examples of alkoxy groups include methoxy, ethoxy, propoxy (e.g., n-propoxy and isopropoxy), t-butoxy, and the like.

The term “alkenyl” when used alone or as part of a substituent group refers to a straight- or branched-chain group having from 2 to 12 carbon atoms (“C₂-C₁₂”), preferably 2 to 6 carbons atoms (“C₂₋₆”), in the group, wherein the group includes at least one carbon-carbon double bond of alkenyl groups include vinyl (—CH═CH₂; C₂alkenyl), allyl (—CH₂— CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (CH═CHCH₂CH₂CH₃ or CH₂═CHCH₂CH₂CH₂—; C₅alkenyl), and the like.

The term “alkynyl” when used alone or as part of a substituent group refers to a straight- or branched-chain group having from 2 to 12 carbon atoms (“C₂-C₁₂”), preferably 2 to 6 carbons atoms (“C₂-C₆”), in the group, wherein the group includes at least one carbon-carbon triple bond. Examples of alkynyl groups include ethynyl (—C≡CH; C₂alkynyl), propargyl (—CH₂—CH≡CH; C₃alkynyl), and the like.

The compounds described herein can be asymmetric (e.g., having one or more stereocenters). All stereoisomers, such as enantiomers and diastereomers, are intended unless otherwise indicated. Compounds provided herein that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present embodiments. Geometric isomers of the compounds of the present embodiments are described and may be isolated as a mixture of isomers or as separated isomeric forms.

Compounds provided herein may also include tautomeric forms. All tautomeric forms are encompassed.

In some embodiments, the compounds may exist as rotational isomers. In some embodiments, the compounds exist as mixtures of rotational isomers in any proportion. In other embodiments, the compounds exist as particular rotational isomers, substantially free of other rotational isomers.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, and salts thereof, are substantially isolated. By “substantially isolated” is meant that the compound is at least partially or substantially separated from the environment in which is formed or detected. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Also provided herein are pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts t include, but are not limited to, the conventional non-toxic salts of the parent compound formed, for example, from non-toxic inorganic or organic acids. The pharmaceutically acceptable salts can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound provided herein with one or more solvent molecules.

Subject” includes humans. The terms “human,” “patient,” and “subject” are used interchangeably herein.

As used herein, the phrase “in need thereof” means that the animal or mammal (subject) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. In some embodiments, the subject in need thereof is suspected of having the condition that needs to be treated.

“Treating” or “treatment” of any disease or disorder refers, in some embodiments, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the subject. In yet another embodiment, “treating” or “treatment” refers to modulating the disease or disorder, either physically (e.g., stabilization of a discernible symptom), physiologically (e.g., stabilization of a physical parameter), or both. In yet another embodiment, “treating” or “treatment” refers to delaying the onset of the disease or disorder.

As used herein, the phrase “integer from X to Y” means any integer that includes the endpoints. For example, the phrase “integer from X to Y or “1-5” or “1 to 5” means 1, 2, 3, 4, or 5 or any value therein if not modified by the term “integer.”

“Compounds of the present disclosure,” and equivalent expressions, are meant to embrace compounds of any formula or structural representation as described herein, as well as their subgenera, which expression includes the stereoisomers (e.g., enantiomers, diastereomers) and constitutional isomers (e.g., tautomers) of the various compounds and formula provided for herein as well as pharmaceutically acceptable salts thereof, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (2H or D), carbon-13 (13C), nitrogen-15 (15N), or the like. It will be understood that, in a compound where such isotopic substitution is made, the following atoms, where present, may vary, so that for example, any hydrogen may be 2H/D, any carbon may be 13C, or any nitrogen may be 15N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well known in the art.

Throughout the description, where compositions are described as having, including, or comprising specific components, or where processes are described as having, including, or comprising specific process steps, it is contemplated that compositions described herein also consist essentially of, or consist of, the recited components, and that the processes described herein also consist essentially of, or consist of, the recited processing steps. Further, it should be understood that the order of steps or order for performing certain actions are immaterial so long as the process remains operable. Moreover, two or more steps or actions can be conducted simultaneously.

In some embodiments, compounds having Formula (I), or pharmaceutically acceptable salts thereof, are provided:

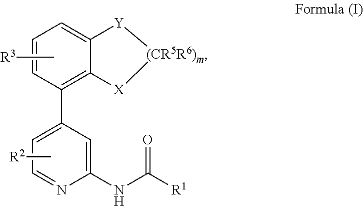

wherein
X is O, S, or CR⁷R⁸;
Y is O, S, CR⁹R¹⁰, or NR⁴;
m is 1-3;
R² and R³ is selected from H, D, halogen, oxo, CN, C₁₋₃ alkyl, C₁₋₃ alkoxy, C₂₋₄ alkenyl, C₂₋₄ alkynyl, C₁₋₃ haloalkyl, and C₁₋₃ haloalkoxy;
R⁴ is selected from H, C₁₋₃ alkyl, C₁₋₃ alkoxy, C₁₋₃ haloalkyl, and —C(O) C₁₋₃ alkyl;
R¹ is selected from C₁₋₆ alkyl, C₁₋₆ alkoxy, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, and (4-14 membered heterocycloalkyl)-C₁₋₄ alkyl;
wherein R¹ is optionally substituted with 1, 2, 3, 4, 5, 6, 7 or 8 independently selected R^{b} substituents;
each R⁵, R⁶, R⁷, R⁸, R⁹, and R¹⁰ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1};
wherein when R², R³, R⁴, R⁵, R⁶, R⁷, R⁸, R⁹, or R¹⁰ is C₁₋₆ alkyl, C₁₋₆ alkoxy, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, or (4-14 membered heterocycloalkyl)-C₁₋₄ alkyl, then R², R³, R⁴, R⁵, R⁶, R⁷, R⁸, R⁹, and R¹⁰ is optionally substituted with 1, 2, 3, 4 or 5 independently selected R^{b} substituents;
optionally R⁵ and R⁶ together with the carbon atom to which they are both attached form a C₄₋₇ spirocyclic ring optionally substituted with 1, 2, 3, 4, or 5 independently selected R^{b} substituents;
optionally R⁴ and R⁵ together with the atoms to which they are attached form a 4-, 5-, 6-, or 7-membered heterocycloalkyl ring optionally substituted with 1, 2, 3, 4, or 5 independently selected R^{b} substituents;
optionally R⁴ and R⁶ together with the atoms to which they are attached form a 4-, 5-, 6-, or 7-membered heterocycloalkyl ring optionally substituted with 1, 2, 3, 4, or 5 independently selected R^{b} substituents;
optionally R⁷ and R⁸ together with the carbon atom to which they are both attached form a C₃-C₇ spirocyclic ring optionally substituted with 1, 2, 3, 4, or 5 independently selected R^{b} substituents,
optionally one of R⁵ and R⁶ and one of R⁷ and R⁸ together with the atoms to which they are attached form a 4-, 5-, 6-, or 7-membered cycloalkyl ring optionally substituted with 1, 2, 3, 4, or 5 independently selected R^{b} substituents; and
optionally one of R⁵ and R⁶ and one of R⁹ and R¹⁰ together with the atoms to which they are attached form a 4-, 5-, 6-, or 7-membered cycloalkyl ring optionally substituted with 1, 2, 3, 4, or 5 independently selected R^{b} substituents; and
wherein when m is 2 or 3, then two R⁵ or two R⁶ together with the atoms to which they are attached optionally form a 4-, 5-, 6-, or 7-membered cycloalkyl or heterocycloalkyl ring optionally substituted with 1, 2, 3, 4 or 5 independently selected R^{b} substituents;
each R^{a1} is independently selected from H, D, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl;
wherein when R^{a1} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a1} is optionally substituted with 1, 2, 3, 4, or 5 independently selected R^{d} substituents;
each R^{b} substituent is independently selected from D, halo, oxo, C₁₋₄ alkyl, C₁₋₆ alkoxy, C₁₋₄ haloalkyl, C₁₋₄ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, NR^{c}S(O)(NR^{c})R^{c}, S(O)NR^{c}R^{c}, S(O)₂R^{c}, and S(O)₂NR^{c}R^{c};
wherein when R^{b} is C₁₋₄ alkyl, C₁₋₆ alkoxy, C₁₋₄ haloalkyl, C₁₋₄ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{b} is optionally substituted with 1, 2, or 3 independently selected R^{d} substituents;
each R^{c} is independently selected from H, D, —OH, C₁₋₆ alkyl, C₁₋₆ alkoxy, C₁₋₄ haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl;
wherein when R^{c} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents;
each R^{f} is independently selected from halogen, C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R^{g}, P(O)(OR^{g})(OR^{g}), S(O)R^{g}, NR^{g}S(O)(NR^{g})R^{g}, S(O)NR^{g}R^{g}, S(O)₂R^{g}, NR^{g}S(O)₂R^{g}, NR^{g} S(O)₂NR^{g}R^{g}, and S(O)₂NR^{g}R^{g};
wherein when R^{f} is C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents;
each Rⁿ is independently selected from C₁₋₄ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, NR^{o}S(O)(NR^{o})R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{g}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, SF₅, P(O)R^{e}R^{e}, P(O)(OR^{e})(OR^{e}), S(O)R^{e}, NR^{e}S(O)(NR^{e})R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e},
wherein when R^{d} is C₁₋₆ alkyl, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents;
each R^{e} is independently selected from H, D, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{e} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents;
each R^{g} is independently selected from H, D, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents;
each R^{P} is independently selected from C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, NR^{r}S(O)(NR^{r})R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r};
each R^{o} or R^{r} is independently selected from H, D, C₁₋₄ alkyl, C₃₋₆ cycloalkyl, C₆₋₁₀ aryl, 5- or 6-membered heteroaryl, C₁₋₄ haloalkyl, C₂₋₄ alkenyl, and C₂₋₄ alkynyl,
wherein when R^{o} or R^{r} is C₁₋₄ alkyl, C₃₋₆ cycloalkyl, C₆₋₁₀ aryl, 5- or 6-membered heteroaryl, C₂₋₄ alkenyl, and C₂₋₄ alkynyl, then R^{o} or R^{r} is optionally substituted with 1, 2, or 3 independently selected R^{q} substituents;
each R^{q} is independently selected from D, OH, CN, —COOH, NH₂, halo, C₁₋₆ alkyl, C₁₋₆ haloalkyl, C₁₋₆ alkoxy, C₁₋₆ haloalkoxy, C₁₋₄ alkylthio, phenyl, 5-6 membered heteroaryl, C₃₋₆ cycloalkyl, 4-6 membered heterocycloalkyl, —CONHR¹¹, —NHC(O)R¹¹, —OC(O)R¹¹, C(O)OR¹¹, —C(O)R¹¹, —SO₂R¹¹, —NHSO₂R¹¹, —SO₂NHR¹¹ and NR¹¹R¹¹,
wherein when R^{q} is C₁₋₆ alkyl, phenyl, 4-6 membered heterocycloalkyl or 5-6 membered heteroaryl, then R^{q} is optionally substituted with OH, CN, —COOH, NH₂, C₁₋₆ alkoxy, C₃₋₆cycloalkyl or 4-6 membered heterocycloalkyl; and
   each R¹¹ is independently C₁₋₆ alkyl.

In some embodiments, compounds having Formula (II-a) or Formula (II-b), or pharmaceutically acceptable salts thereof, are provided:

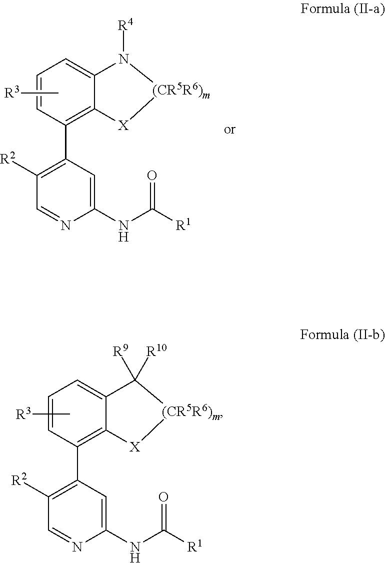

wherein m, R¹, R², R³, R⁴, R⁵, R⁶, R⁹, and R¹⁰ are as defined herein and above.

In some embodiments, X is O, S, or CR⁷R⁸. In some embodiments, X is O. In some embodiments, X is S. In some embodiments, X is CR⁷R⁸.

In some embodiments, m is 1. In some embodiments, m is 2. In some embodiments, m is 3.

In some embodiments, Y is O, CR⁹R¹⁰, or NR⁴. In some embodiments, Y is S. In some embodiments, Y is O. In some embodiments, Y is CR⁹R¹⁰. In some embodiments, Y is NR⁴.

In some embodiments, compounds having Formula (III-a) or Formula (III-b), or pharmaceutically acceptable salts thereof, are provided:

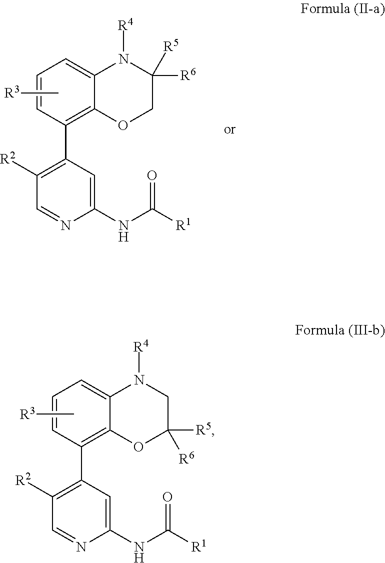

wherein R¹, R², R³, R³, R⁴, R⁵, and R⁶ are as defined herein and above.

In some embodiments, R⁵ and R⁶ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1} In some embodiments, R⁵ and R⁶ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, and NO₂. In some embodiments, R⁵ and R⁶ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, and C₃₋₁₀ cycloalkyl. In some embodiments, R⁵ and R⁶ are independently H or C₁₋₆ alkyl. In some embodiments, R⁵ and R⁶ are independently C₁₋₆ alkyl. In some embodiments, R⁵ and R⁶ are CH₃. In some embodiments, R⁵ is H.

In some embodiments, compounds having Formula (IV-a), Formula (IV-b), Formula (IV-c), or Formula (IV-d), or pharmaceutically acceptable salts thereof, are provided:

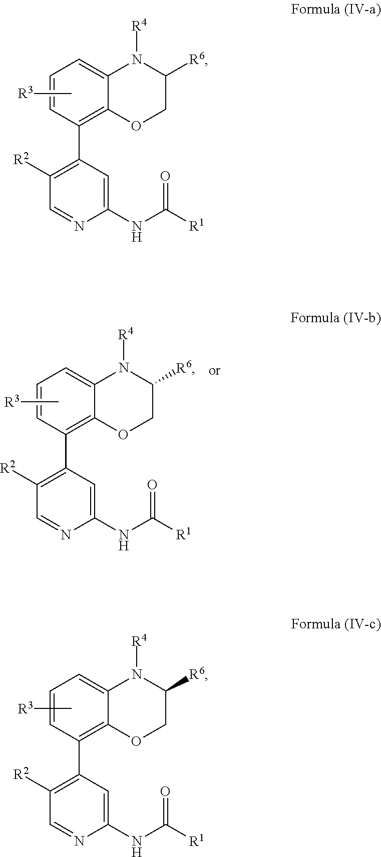

wherein R¹, R², R³, R⁴, and R⁶ are as defined herein and above.

In some embodiments, R⁶ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1}. In some embodiments, R⁶ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, (4-14 membered heterocycloalkyl)-C₁₋₄ alkyl, CN, and NO₂. In some embodiments, R⁶ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆haloalkoxy, C₆₋₁₀ aryl, and C₃₋₁₀ cycloalkyl. In some embodiments, R⁶ is H or C₁₋₆ alkyl. In some embodiments, R⁶ is H. In some embodiments, R⁶ is C₁₋₆ alkyl. In some embodiments, R⁶ is CH₃.

In some embodiments, compounds having Formula (V), or a pharmaceutically acceptable salt thereof, are provided:

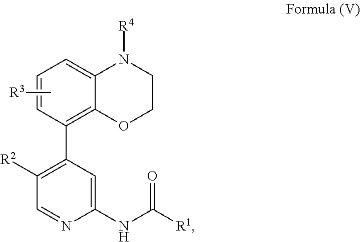

wherein R¹, R², R³, and R⁴ are as defined herein and above.

In some embodiments, R¹ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1}. In some embodiments, R¹ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-14 membered heteroaryl)-C₁₋₄ alkyl, (4-14 membered heterocycloalkyl)-C₁₋₄ alkyl, CN, and NO₂. In some embodiments, R¹ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆haloalkoxy, C₆₋₁₀ aryl, and C₃₋₁₀ cycloalkyl.

In some embodiments, R¹ is C₃₋₁₀ cycloalkyl optionally substituted with 1, 2, 3, or 4 independently selected R^{b} substituents. In some embodiments, R¹ is C₃₋₁₀ cycloalkyl optionally substituted with 1 R^{b} substituent. In some embodiments, R¹ is C₃₋₁₀ cycloalkyl optionally substituted with 2 independently selected R^{b} substituents. In some embodiments, R¹ is C₃₋₁₀ cycloalkyl optionally substituted with 3 independently selected R^{b} substituents. In some embodiments, R¹ is C₃₋₁₀ cycloalkyl optionally substituted with 4 independently selected R^{b} substituents.

In some embodiments, R¹ is C₃₋₇ cycloalkyl optionally substituted with 1, 2, 3, or 4 independently selected R^{b} substituents. In some embodiments, R¹ is C₃₋₇ cycloalkyl optionally substituted with 1 R^{b} substituent. In some embodiments, R¹ is C₃₋₇ cycloalkyl optionally substituted with 2 independently selected R^{b} substituents. In some embodiments, R¹ is C₃₋₇ cycloalkyl optionally substituted with 3 independently selected R^{b} substituents. In some embodiments, R¹ is C₃₋₇ cycloalkyl optionally substituted with 4 independently selected R^{b} substituents.

In some embodiments, R¹ is C₅₋₆ cycloalkyl optionally substituted with 1, 2, 3, or 4 independently selected R^{b} substituents. In some embodiments, R¹ is C₅₋₆ cycloalkyl optionally substituted with 1 R^{b} substituent. In some embodiments, R¹ is C₅₋₆ cycloalkyl optionally substituted with 2 independently selected R^{b} substituents. In some embodiments, R¹ is C₅₋₆ cycloalkyl optionally substituted with 3 independently selected R^{b} substituents. In some embodiments, R¹ is C₅₋₆ cycloalkyl optionally substituted with 4 independently selected R^{b} substituents. In some embodiments, R¹ is

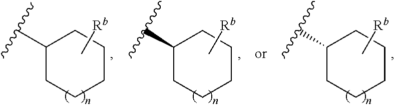

wherein n is 0 or 1 and R^{b} is as defined herein and above. In some embodiments, R¹ is

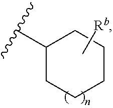

wherein n is 0 or 1 and R^{b} is as defined herein and above. In some embodiments, R¹ is

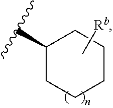

wherein n is 0 or 1 and R^{b} is as defined herein and above. In some embodiments, R¹ is

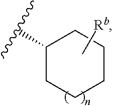

wherein n is 0 or 1 and R^{b} is as defined herein and above.

In some embodiments, R¹ is

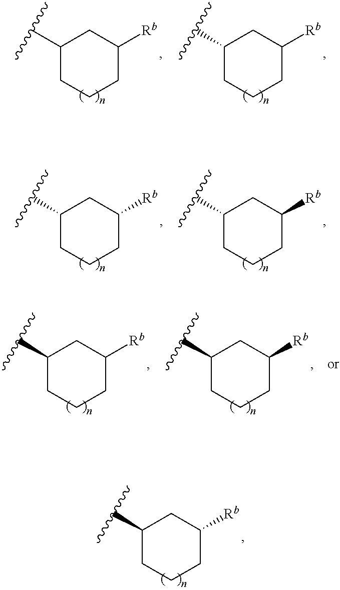

wherein n is 0 or 1 and R^{b} is as defined herein and above. In some embodiments, R¹ is

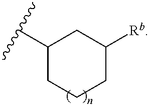

In some embodiments, R¹ is

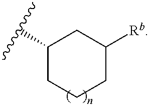

In some embodiments, R¹ is

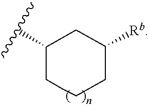

In some embodiments, R¹ is

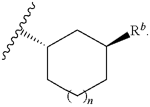

In some embodiments, R¹ is

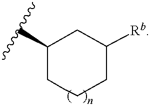

In some embodiments, R¹ is

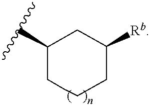

In some embodiments, R¹ is or

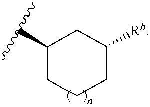

In some embodiments, R^{b} is NR^{c}C(O)R^{c}, NR^{c}C(O)NR^{c}R^{c}, NR^{c}S(O)(NR^{c})R^{c}, or NR^{c}S(O)₂NR^{c}R^{c}. In some embodiments, R^{b} is NR^{c}C(O)R^{c}. In some embodiments, R^{b} is NR^{c}C(O)NR^{c}R^{c}. In some embodiments, R^{b} is NR^{c}S(O)₂NR^{c}R^{c}.

In some embodiments, R^{c} in NR^{c}C(O)R^{c}, NR^{c}C(O)NR^{c}R^{c}, NR^{c}S(O)(NR^{c})R^{c}, or NR^{c}S(O)₂NR^{c}R^{c} is independently H, C₁₋₆ alkyl, C₃₋₁₀ cycloalkyl, 4-10 membered heterocycloalkyl, or (5-10 membered heteroaryl)-C₁₋₄ alkyl; wherein when R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, then R^{c} is optionally substituted with 1, 2, 3, 4, or 5 independently selected R^{f} substituents. In some embodiments, R^{c} is independently H, C₁₋₆ alkyl, C₃₋₁₀ cycloalkyl, 4-10 membered heterocycloalkyl, or (5-10 membered heteroaryl)-C₁₋₄ alkyl. In some embodiments, R^{c} is independently H. In some embodiments, R^{c} is C₁₋₆ alkyl. In some embodiments, R^{c} is C₃₋₁₀ cycloalkyl. In some embodiments, R^{c} is 4-10 membered heterocycloalkyl. In some embodiments, R^{c} is (5-10 membered heteroaryl)-C₁₋₄ alkyl. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 1, 2, 3, 4, or 5 independently selected R^{f} substituents. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 1 R^{f} substituent. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 2 independently selected R^{f} substituents. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 3 independently selected R^{f} substituents. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 4 independently selected R^{f} substituents. In some embodiments, R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, each of which is optionally substituted with 5 independently selected R^{f} substituents.

In some embodiments, each R^{f} is independently selected from halogen, C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R^{g}, P(O)(OR^{g})(OR^{g}), S(O)R^{g}, NR^{g}S(O)(NR^{g})R^{g}, S(O)NR^{g}R^{g}, S(O)₂R^{g}, NR^{g}S(O)₂R^{g}, NR^{g} S(O)₂NR^{g}R^{g}, and S(O)₂NR^{g}R^{g}. In some embodiments, each R^{f} substituents are independently halogen, CN or OR^{g}. In some embodiments, each R^{f} substituents are independently halogen. In some embodiments, R^{f} is CN. In some embodiments, each R^{f} substituents are independently OR^{g}.

In some embodiments, each R^{g} is independently selected from H, D, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl. In some embodiments, R^{g} is H. In some embodiments, each R^{g} is independently C₁₋₆ alkyl.

In some embodiments, R^{b} is acetamido (—NHC(O)CH₃), 3-hydroxybutanamido (—NHC(O)CH₂CH(OH)CH₃), propionamido (—NHC(O)CH₂CH₃), 2-methoxyacetamido (—NHC(O)CH₂—OCH₃), 2-cyanoacetamido (—NHC(O)CH₂—CN), 1-hydroxycyclopropane-1-carboxamido,

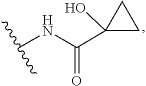 [SMILES: *NC(=O)C1(O)CC1]

2-(thiazol-4-yl)acetamido,

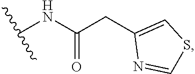 [SMILES: *NC(=O)Cc1cscn1]

methylsulfonamido (—NSO₂CH₃), 3-methylureido (—NC(O)NHCH₃), 3-methoxyureido (—NC(O)NHOCH₃), 3,3-dimethylureido (—NC(O)N(CH₃)₂), or 3-ethylureido (—NC(O)NHCH₂CH₃), morpholine-4-carboxamido, i.e.,

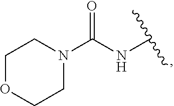 [SMILES: *NC(=O)N1CCOCC1]

or 4-methylpiperazine-1-carboxamide, i.e.,

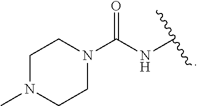 [SMILES: *NC(=O)N1CCN(C)CC1]

In some embodiments, R^{b} is acetamido (—NHC(O)CH₃). In some embodiments, R^{b} is 3-hydroxybutanamido (—NHC(O)CH₂CH(OH)CH₃). In some embodiments, R^{b} is propionamido (—NHC(O)CH₂CH₃). In some embodiments, R^{b} is 2-methoxyacetamido (—NHC(O)CH₂—OCH₃). In some embodiments, R^{b} is 2-cyanoacetamido (—NHC(O)CH₂—CN), 1-hydroxycyclopropane-1-carboxamido. In some embodiments, R^{b} is

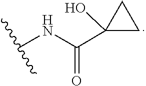 [SMILES: *NC(=O)C1(O)CC1]

In some embodiments, R^{b} is 2-(thiazol-4-yl)acetamido. In some embodiments, R^{b} is

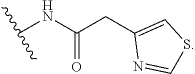 [SMILES: *NC(=O)Cc1cscn1]

In some embodiments, R^{b} is methylsulfonamido (—NSO₂CH₃). In some embodiments, R^{b} is 3-methylureido (—NC(O)NHCH₃), 3-methoxyureido (—NC(O)NHOCH₃). In some embodiments, R^{b} is 3,3-dimethylureido (—NC(O)N(CH₃)₂). In some embodiments, R^{b} is 3-ethylureido (—NC(O)NHCH₂CH₃). In some embodiments, R^{b} is morpholine-4-carboxamido, i.e.,

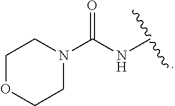 [SMILES: *NC(=O)N1CCOCC1]

In some embodiments, R^{b} is 4-methylpiperazine-1-carboxamide, i.e.,

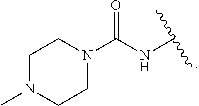 [SMILES: *NC(=O)N1CCN(C)CC1]

In some embodiments, compounds having Formula (VI), or a pharmaceutically acceptable salt thereof, are provided:

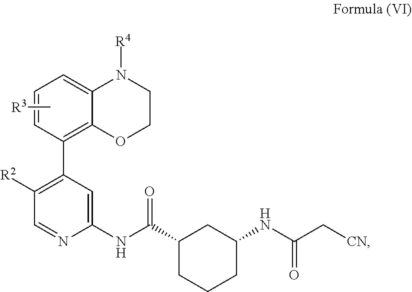

wherein R², R³, and R⁴ are as defined herein and above.

In some embodiments, compounds having Formula (VII), or a pharmaceutically acceptable salt thereof, are provided:

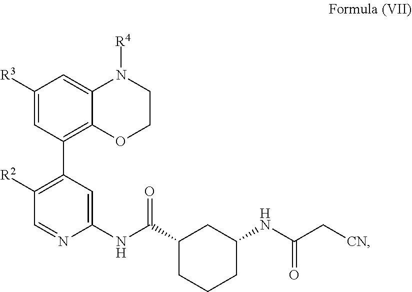

wherein R², R³, and R⁴ are as defined herein and above.

In some embodiments, R² is selected from H, D, halo, oxo, C₁₋₆ alkyl, C₁₋₆ alkoxy, C₂₋₆ alkenyl, C₂₋₆ alkynyl, C₁₋₆haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1}. In some embodiments, R² is selected from H, D, halo, oxo, C₁₋₆ alkyl, C₁₋₆ alkoxy, C₂₋₆ alkenyl, C₂₋₆ alkynyl, C₁₋₆haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, and NO₂. In some embodiments, R² is selected from H, D, halo, oxo, C₁₋₆ alkyl, C₁₋₆ alkoxy, C₂₋₆ alkenyl, C₂₋₆ alkynyl, C₁₋₆haloalkyl, C₁₋₆ haloalkoxy, C₆₋₁₀ aryl, and C₃₋₁₀ cycloalkyl. In some embodiments, R² is H, halogen, or C₁₋₆ alkyl. In some embodiments, R² is H or halogen. In some embodiments, R² is H. In some embodiments, R² is halogen. In some embodiments, R² is Cl. In some embodiments, R² is C₁₋₆ alkyl. In some embodiments, R² is CH₃.

In some embodiments, R³ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1}. In some embodiments, R³ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, and NO₂. In some embodiments, R³ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, and C₃₋₁₀ cycloalkyl. In some embodiments, R³ is H, halogen, or C₁₋₆ alkyl. In some embodiments, R² is H or halogen. In some embodiments, R³ is H. In some embodiments, R³ is halogen. In some embodiments, R³ is F.

In some embodiments, compounds having Formula (VIII), or a pharmaceutically acceptable salt thereof, are provided:

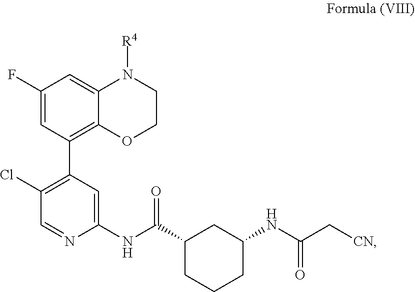

wherein R⁴ is as defined herein and above.

In some embodiments, R⁴ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1} S(O)₂R^{a1}, SF₅, P(O)R^{a1}R^{a1}, P(O)(OR^{a1})(OR^{a1}), B(OR^{a1})₂, and S(O)₂NR^{a1}R^{a1}. In some embodiments, R⁴ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, and NO₂. In some embodiments, R⁴ is selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, and C₃₋₁₀ cycloalkyl. In some embodiments, R⁴ is H, C₁₋₆ alkyl, or C₁₋₆ alkoxy. In some embodiments, R⁴ is H. In some embodiments, R⁴ is C₁₋₆ alkyl. In some embodiments, R⁴ is C₁₋₆ alkoxy.

In some embodiments, compounds having Formula (IX-b), or a pharmaceutically acceptable salt thereof, are provided:

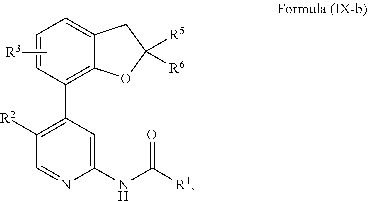

wherein R¹, R², R³, R⁵, and R⁶ are as defined herein and above.

In some embodiments, R⁵ and R⁶ are independently H or C₁₋₆ alkyl. In some embodiments, R⁵ and R⁶ are H. In some embodiments, R⁵ and R⁶ are independently C₁₋₆ alkyl. In some embodiments, R⁵ and R⁶ are CH₃.

In some embodiments, compounds having Formula (X), or a pharmaceutically acceptable salt thereof, are provided:

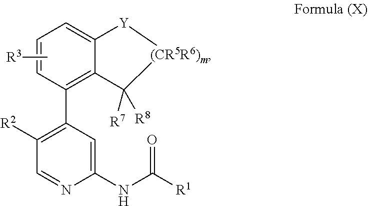

wherein Y, m, R¹, R², R³, R⁵, R⁶, R⁷, and R⁸ are as defined herein and above.

In some embodiments, compounds having Formula (XII), or a pharmaceutically acceptable salt thereof, are provided:

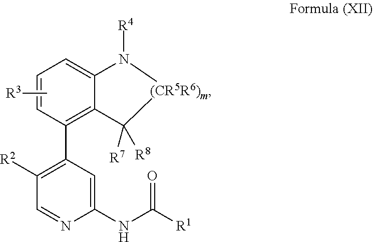

wherein m, R¹, R², R³, R⁴, R⁵, R⁶, R⁷ and R⁸ are as defined herein and above. In some embodiments, m is 2.

In some embodiments, compounds having Formula (XII), or a pharmaceutically acceptable salt thereof, are provided:

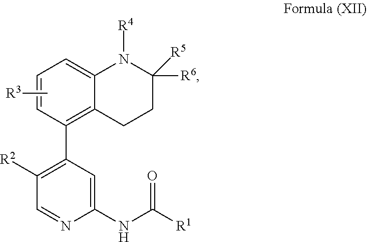

wherein R¹, R², R³, R⁴, R⁵, and R⁶ are as defined herein and above. In some embodiments, R⁵ and R⁶ are independently H or C₁₋₆ alkyl. In some embodiments, R⁵ and R⁶ are H.

In some embodiments, compounds having Formula (XIII-a) or Formula (XIII-b), or a pharmaceutically acceptable salt thereof, are provided:

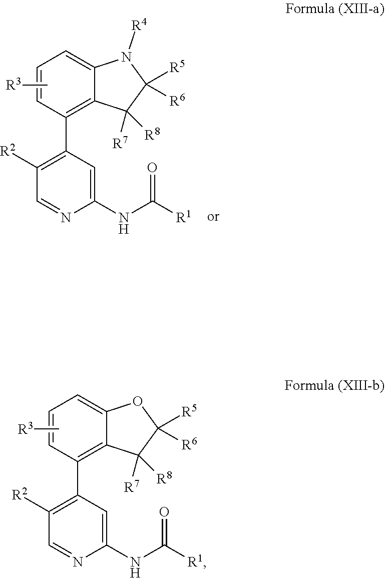

wherein R¹, R², R³, R⁴, R⁵, R⁶, R⁷, and R⁸ are as defined herein and above. In some embodiments, R⁵, R⁶, R⁷′ and R⁸ are independently H. In some embodiments, R⁵, R⁶, R⁷′ and R⁸ are independently C₁₋₆ alkyl. In some embodiments, R⁷ and R⁸ are H. In some embodiments, R⁵ is H. In some embodiments, R⁵ and R⁶ are C₁₋₆ alkyl. In some embodiments, R⁵ and R⁶ are CH₃.

In some embodiments, R¹ is C₃₋₁₀ cycloalkyl optionally substituted with 1, 2, 3, or 4 independently selected R^{b} substituents. In some embodiments, R¹ is C₃₋₇ cycloalkyl optionally substituted with 1, 2, 3, or 4 independently selected R^{b} substituents. In some embodiments, wherein R¹ is C₅₋₆ cycloalkyl optionally substituted with 1, 2, 3, or 4 independently selected R^{b} substituents. In some embodiments, R¹ is

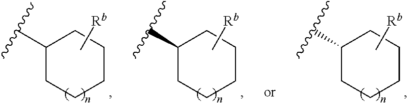

wherein n is 0 or 1 and R^{b} is as defined herein and above. In some embodiments, R¹ is

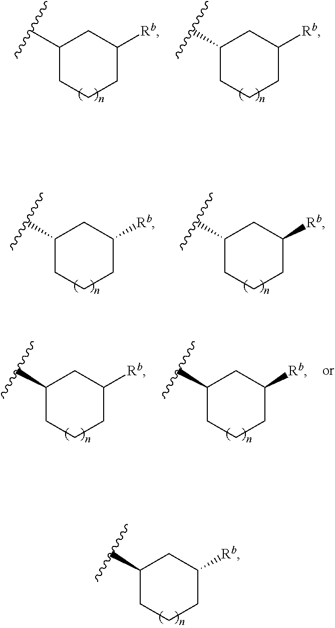

wherein n is 0 or 1 and R^{b} is as defined in claims 1-9. In some embodiments, R^{b} is NR^{c}C(O)R^{c}, NR^{c}C(O)NR^{c}R^{c}, NR^{c}S(O)(NR^{c})R^{c}, or NR^{c}S(O)₂NR^{c}R^{c}. In some embodiments, R^{c} in NR^{c}C(O)R^{c}, NR^{c}C(O)NR^{c}R^{c}, or NR^{c}S(O)₂NR^{c}R^{c} is independently H, C₁₋₆ alkyl, C₃₋₁₀ cycloalkyl, 4-10 membered heterocycloalkyl, or (5-10 membered heteroaryl)-C₁₋₄ alkyl; wherein when R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, then R^{c} is optionally substituted with 1, 2, 3, 4, or 5 independently selected R^{f} substituents. In some embodiments, R^{f} substituents are independently halogen, CN, or OR^{g}. In some embodiments, R^{g} is independently H or C₁₋₆ alkyl. In some embodiments, R^{b} is acetamido (—NHC(O)CH₃), 3-hydroxybutanamido (—NHC(O)CH₂CH(OH)CH₃), propionamido (—NHC(O)CH₂CH₃), 2-methoxyacetamido (—NHC(O)CH₂—OCH₃), 2-cyanoacetamido (—NHC(O)CH₂—CN), 1-hydroxycyclopropane-1-carboxamido,

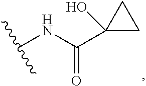 [SMILES: *NC(=O)C1(O)CC1]

2-(thiazol-4-yl)acetamido,

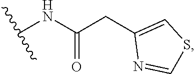 [SMILES: *NC(=O)Cc1cscn1]

methylsulfonamido (—NSO₂CH₃), 3-methylureido (—NC(O)NHCH₃), 3-methoxyureido (—NC(O)NHOCH₃), 3,3-dimethylureido (—NC(O)N(CH₃)₂), or 3-ethylureido (—NC(O)NHCH₂CH₃), morpholine-4-carboxamido, i.e.,

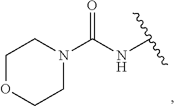 [SMILES: *NC(=O)N1CCOCC1]

or 4-methylpiperazine-1-carboxamide, i.e.,

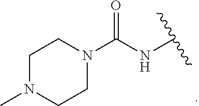 [SMILES: *NC(=O)N1CCN(C)CC1]

In some embodiments, R² is H, halogen, or C₁₋₆ alkyl. In some embodiments, R² is halogen. In some embodiments, R² is Cl. In some embodiments, R³ is H, halogen, or C₁₋₆ alkyl R³ is H. In some embodiments, R⁴ is H.

In some embodiments, provided are compounds selected from the group consisting of:

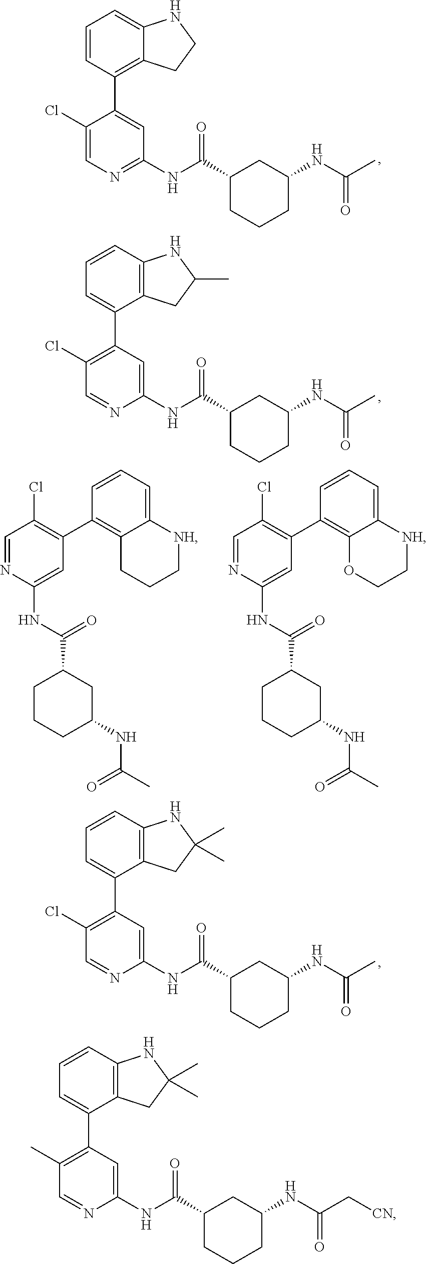

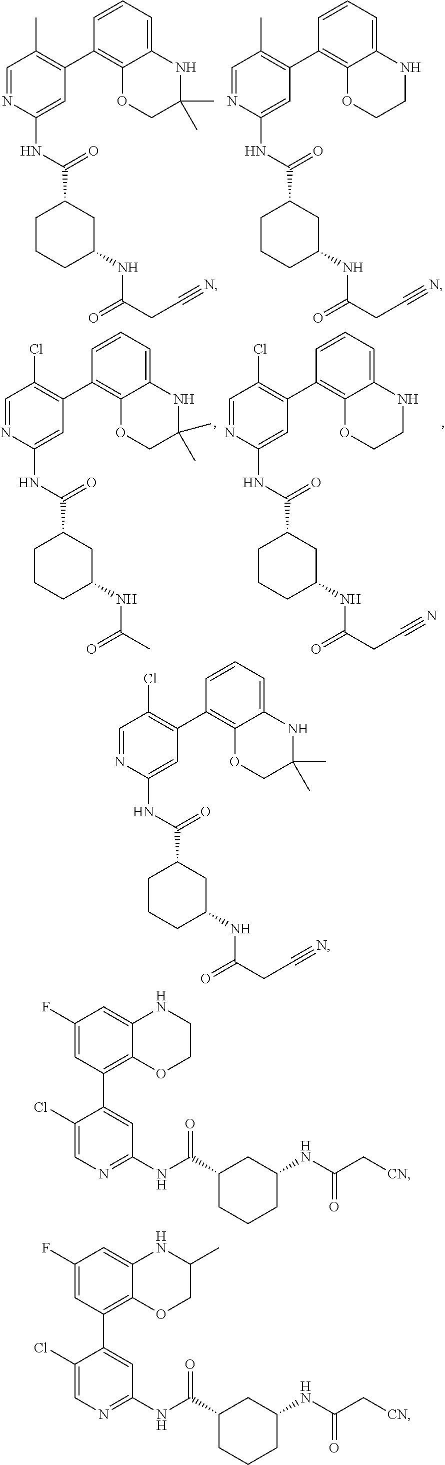

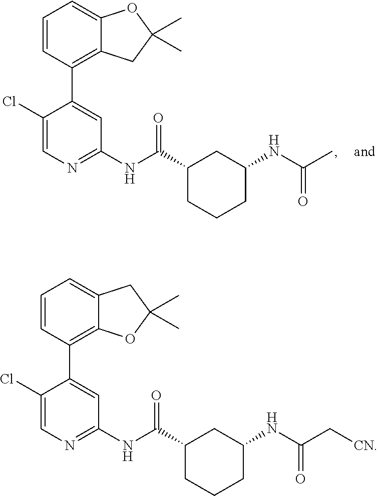 [SMILES: CC(=O)N[C@@H]1CCC[C@H](C(=O)Nc2cc(-c3cccc4c3CC(C)(C)O4)c(Cl)cn2)C1 ; CC1(C)Cc2cccc(-c3cc(NC(=O)[C@H]4CCC[C@@H](NC(=O)CC#N)C4)ncc3Cl)c2O1]

In some embodiments, provided are compounds selected from the group consisting of:
(1S,3R)-3-acetamido-N-(5-chloro-4-(indolin-4-yl)pyridin-2-yl)cyclohexane-1-carboxamide;
(1S,3R)-3-acetamido-N-(5-chloro-4-(2-methylindolin-4-yl)pyridin-2-yl)cyclohexane-1-carboxamide;
(1S,3R)-3-acetamido-N-(5-chloro-4-(1,2,3,4-tetrahydroquinolin-5-yl)pyridin-2-yl)cyclohexane-1-carboxamide;
(1S,3R)-3-acetamido-N-(5-chloro-4-(3,4-dihydro-2H-benzo[b][1,4]oxazin-8-yl)pyridin-2-yl)cyclohexane-1-carboxamide;
(1S,3R)-3-acetamido-N-(5-chloro-4-(2,2-dimethylindolin-4-yl)pyridin-2-yl)cyclohexane-1-carboxamide;
(1S,3R)-3-(2-cyanoacetamido)-N-(4-(2,2-dimethylindolin-4-yl)-5-methylpyridin-2-yl)cyclohexane-1-carboxamide;
(1S,3R)-3-(2-cyanoacetamido)-N-(4-(3,3-dimethyl-3,4-dihydro-2H-benzo[b][1,4]oxazin-8-yl)-5-methylpyridin-2-yl)cyclohexane-1-carboxamide;
(1S,3R)-3-(2-cyanoacetamido)-N-(4-(3,4-dihydro-2H-benzo[b][1,4]oxazin-8-yl)-5-methylpyridin-2-yl)cyclohexane-1-carboxamide;
(1S,3R)-3-acetamido-N-(5-chloro-4-(3,3-dimethyl-3,4-dihydro-2H-benzo[b][1,4]oxazin-8-yl)pyridin-2-yl)cyclohexane-1-carboxamide;
(1S,3R)—N-(5-chloro-4-(3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(3,3-dimethyl-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3-methyl-3,4-dihydro-2H-benzo[b][1,4]oxazin-8-yl)pyridin-2-yl)-3-(2-cyanoacetamido)cyclohexane-1-carboxamide;
(1S,3R)-3-acetamido-N-(5-chloro-4-(2,2-dimethyl-2,3-dihydrobenzofuran-4-yl)pyridin-2-yl)cyclohexane-1-carboxamide; and
(1S,3R)—N-(5-chloro-4-(2,2-dimethyl-2,3-dihydrobenzofuran-7-yl)pyridin-2-yl)-3-(2-cyanoacetamido)cyclohexane-1-carboxamide.

In some embodiments, also provided are pharmaceutical compositions comprising one or more compounds as provided or described herein, or a pharmaceutically acceptable salt or solvate thereof, and optionally a pharmaceutically acceptable excipient. In some embodiments, also provided are pharmaceutical compositions comprising one or more compounds as provided or described herein, or a pharmaceutically acceptable salt or solvate thereof, and a pharmaceutically acceptable excipient.

In some embodiments, the pharmaceutical composition comprises an enantiomeric excess of at least 90% of one enantiomer of the compound, or a pharmaceutically acceptable salt or solvate thereof. In some embodiments, the pharmaceutical composition comprises an enantiomeric excess of at least 95% of one enantiomer of the compound, or a pharmaceutically acceptable salt or solvate thereof. In some embodiments, the pharmaceutical composition comprises an enantiomeric excess of at least 98% of one enantiomer of the compound, or a pharmaceutically acceptable salt or solvate thereof. In some embodiments, the pharmaceutical composition comprises an enantiomeric excess of at least 99% of one enantiomer of the compound, or a pharmaceutically acceptable salt or solvate thereof.

In some embodiments, provided are methods of inhibiting a CDK enzyme comprising: contacting the CDK enzyme with an effective amount of one or more compounds as provided or described herein, or a pharmaceutically acceptable salt or solvate thereof, or a pharmaceutical composition as provided or described herein. In some embodiments, the CDK enzyme is CDK9.

In some embodiments, provided are methods of treating a disease or disorder associated with aberrant CDK activity in a subject or a subject in need thereof comprising administering to the subject, one or more compounds as provided or described herein, or a pharmaceutically acceptable salt or solvate thereof, or a pharmaceutical composition as provided or described herein. In some embodiments, the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

In some embodiments, provided are methods of treating cancer in a subject or a subject in need thereof comprising administering to the subject, a compound of one or more compounds as provided or described herein, or a pharmaceutically acceptable salt or solvate thereof. In some embodiments, the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

In some embodiments, wherein the subject is a subject in need thereof. In some embodiments, wherein the therapeutic for treating a disease or disorder associated with aberrant CDK activity is selected from those described herein. In some embodiments, the therapeutic for treating cancer.

In some embodiments, the condition is prevented.

It will be apparent that the compounds provided herein, including all subgenera described herein, may have multiple stereogenic centers. As a result, there exist multiple stereoisomers (enantiomers and diastereomers) of the compounds of the various formula provided herein (and subgenera provided herein). The present disclosure contemplates and encompasses each stereoisomer of any compound of any formula provided herein (and subgenera provided herein), as well as mixtures of said stereoisomers. All enantiomers, diastereomers, and mixtures thereof, are included within the scope of compounds described herein.

Pharmaceutically acceptable salts and solvates of the compounds of any formula provided herein (including all subgenera provided herein) are also within the scope of the disclosure. Isotopic variants of the compounds of any formula provided herein (including all subgenera provided herein) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate, or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds and other agent(s) may be mixed into a preparation or both components may be formulated into separate preparations to use them in combination separately or at the same time.

In some embodiments, the concentration of one or more compounds provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, a pharmaceutical composition comprising the R enantiomer is free or substantially free of the S enantiomer.

In some embodiments, a pharmaceutical composition comprising the S enantiomer is free or substantially free of the R enantiomer.

In some embodiments, a pharmaceutical composition comprises an enantiomeric excess of at least, or about, 80, 85, 90, 91, 92, 93, 94, 95, 96, 97, 98, 99% of a specific enantiomer of a compound provided herein, such as the R or the S enantiomer. In some embodiments, the enantiomeric excess is at least, or about 90%. In some embodiments, the enantiomeric excess is at least, or about 95%. In some embodiments, the enantiomeric excess is at least, or about 98%. In some embodiments, the enantiomeric excess is at least, or about 99%.

The compounds can be effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition can contain an active ingredient (i.e., a compound of the disclosure) provided for herein or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, pharmaceutical compositions for oral administration are provided that contain a compound provided herein, and a pharmaceutical excipient suitable for oral administration.

In some embodiments, embodiments provide a solid pharmaceutical composition for oral administration containing: (i) an amount (e.g., effective amount) of a compound; optionally (ii) an amount of a second agent; and (iii) a pharmaceutical excipient suitable for oral administration. In some embodiments, the composition further contains: (iv) an amount of a third agent.

In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Embodiments provided for herein further encompass anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms that contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrose, kaolin, mannitol, silicic acid, sorbitol, starch, pre-gelatinized starch, and mixtures thereof.

Disintegrants may be used in the compositions provided for herein to provide tablets that disintegrate when exposed to an aqueous environment. Too much of a disintegrant may produce tablets, which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants that can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants that can be used to form pharmaceutical compositions and dosage forms include, but are not limited to hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic and have greater solubility in oils, while surfactants with higher HLB values are more hydrophilic and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-pho sphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some embodiments, the composition may include a solubilizer to ensure good solubilization and/or dissolution of the compound(s) and to minimize precipitation of the compound(s). This can be used, for example, for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, 6-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject or a subject in need thereof using conventional techniques, such as distillation or evaporation. Thus, if present, the solubilizer can be in a weight ratio of 10%, 25% o, 50%), 100% o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

[01.85] In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, pharmaceutical compositions for injection are provided containing a compound and a pharmaceutical excipient suitable for injection. Components and amounts of agents in the compositions are as described herein.

The forms in which the compositions may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound(s) in an amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques, which yield a powder of the active ingredient plus any additional desired ingredient from a previously sterile-filtered solution thereof.

Pharmaceutical Compositions for Topical (e.g. Transdermal) Delivery.

In some embodiments, pharmaceutical compositions for transdermal delivery are provided containing a compound(s) and a pharmaceutical excipient suitable for transdermal delivery.

Compositions can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods employs transdermal delivery devices (“patches”). Such transdermal patches may be used to provide continuous or discontinuous infusion of a compound in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effects. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical compositions can be effected by any method that enables delivery of the compounds to the site of action. These methods include oral routes, intraduodenal routes, parenteral injection (including intravenous, intraarterial, subcutaneous, intramuscular, intravascular, intraperitoneal, or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound, and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases, still larger doses may be employed without causing any harmful side effect, e.g., by dividing such larger doses into several small doses for administration throughout the day.

In some embodiments, a compound is administered in a single dose.

Typically, such administration can be by injection, e.g., intravenous injection, in order to introduce the agent quickly. However, other routes, such as oral, may be used as appropriate. A single dose of a compound may also be used for treatment of an acute condition.

In some embodiments, a compound is administered in multiple doses. Dosing may be about once, twice, three times, four times, five times, six times, or more than six times per day. Dosing may be about once a month, once every two weeks, once a week, or once every other day. In some embodiments a compound and another agent are administered together about once per day to about 6 times per day. In some embodiments, the administration of a compound and an agent continues for less than about 7 days. In yet another embodiment, the administration continues for more than about 6, 10, 14, 28 days, two months, six months, or one year. In some cases, continuous dosing is achieved and maintained as long as necessary.

Administration of the compounds may continue as long as necessary. In some embodiments, a compound is administered for more than 1, 2, 3, 4, 5, 6, 7, 14, or 28 days. In some embodiments, a compound is administered for less than 28, 14, 7, 6, 5, 4, 3, 2, or 1 day. In some embodiments, a compound is administered chronically on an ongoing basis, e.g., for the treatment of chronic effects.

A compound may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds may slow or inhibit the migration and proliferation of smooth muscle cells in the arterial wall, which contribute to restenosis. A compound may be administered, for example, by local delivery from the struts of a stent, from a stent graft, from grafts, or from the cover or sheath of a stent. In some embodiments, a compound is admixed with a matrix. Such a matrix may be a polymeric matrix and may serve to bond the compound to the stent. Polymeric matrices suitable for such use include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be non-degrading or may degrade with time, releasing the compound or compounds. Compounds may be applied to the surface of the stent by various methods such as dip/spin coating, spray coating, dip-coating, and/or brush-coating. The compounds may be applied in a solvent and the solvent may be allowed to evaporate, thus forming a layer of the compound onto the stent. Alternatively, the compound may be located in the body of the stent or graft, for example, in microchannels or micropores. When implanted, the compound diffuses out of the body of the stent to contact the arterial wall. Such stents may be prepared by dipping a stent manufactured to contain such micropores or microchannels into a solution of the compound in a suitable solvent, followed by evaporation of the solvent. Excess drugs on the surface of the stent may be removed via an additional brief solvent wash. In yet other embodiments, compounds may be covalently linked to a stent or graft. A covalent linker may be used, which degrades in vivo, leading to the release of the compound. Any bio-labile linkage may be used for such a purpose, such as an ester, amide or anhydride linkages. Compounds may additionally be administered intravascularly from a balloon used during angioplasty. Extravascular administration of the compounds via the pericardial or via adventitial application of formulations may also be performed to decrease restenosis.

A variety of stent devices, which may be used as described, are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds may be administered in dosages. It is known in the art that due to intersubject variability in compound pharmacokinetics, individualization of dosing regimen is necessary for optimal therapy. Dosing for a compound may be found by routine experimentation in light of the instant disclosure.

When a compound is administered in a composition that comprises one or more agents, which has a shorter half-life than the compound unit dose forms of the agent and the compound may be adjusted accordingly.

The subject pharmaceutical composition may, for example, be in a form suitable for oral administration as a tablet, capsule, pill, powder, sustained-release formulations, solution, and suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition can include a conventional pharmaceutical carrier or excipient and a compound as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

In some embodiments, the method comprises administering to a subject or a subject in need thereof an amount, such as a therapeutically effective amount, of a compound or a pharmaceutically acceptable salt or solvate thereof. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC50” refers to the half-maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor, or microorganism) by half. In other words, it is the half-maximal (50%) inhibitory concentration (IC) of a substance (50% IC, or IC50). EC50 refers to the plasma concentration required for obtaining 50%> of a maximum effect in vivo.

In some embodiments, the subject methods utilize a CDK inhibitor with an IC50 value of about or less than a predetermined value, as ascertained in an in vitro assay. In some embodiments, the CDK inhibitor inhibits CDK with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above). In some embodiments, the CDK enzyme is CDK9.

In some embodiments, the subject method of inhibiting CDK enzyme comprises contacting the CDK enzyme with an effective amount of a compound or a pharmaceutically acceptable salt thereof as described herein. In some embodiments, the CDK enzyme is CDK9.

In some embodiments, the CDK inhibitor selectively inhibits CDK with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other CDKs. In some embodiments, the CDK inhibitor is a CDK9 inhibitor.

In some embodiments, the CDK inhibitor selectively inhibits CDK with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 50011M (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other CDKs. In some embodiments, the CDK inhibitor is a CDK9 inhibitor.

In some embodiments, compounds described herein are in use for inhibiting a CDK enzyme in a subject, wherein the use comprises administering to the subject an effective amount of one or more compounds as described herein, pharmaceutically acceptable salts, solvates, pharmaceutical compositions, or prodrugs thereof.

In some embodiments, are provided pharmaceutical compositions as described herein are in use for inhibiting a CDK enzyme in a subject, wherein the use comprises administering to the subject an effective amount of one or more pharmaceutical compositions as described

In some embodiments, are provided uses of compounds as described herein in the manufacture of a formulation inhibiting a CDK enzyme in a subject, wherein the use comprises administering to the subject an effective amount of one or more compounds as described herein, pharmaceutically acceptable salts, solvates, pharmaceutical compositions, or prodrugs thereof.

In some embodiments, are provided uses of a pharmaceutical composition as described herein for inhibiting a CDK enzyme in a subject, wherein the use comprises administering to the subject an effective amount of one or more pharmaceutical compositions as described herein. In some embodiments, the CDK enzyme is CDK9.

The subject methods are useful for treating a disease or disorder condition associated with CDK. Any disease or disorder condition that results directly or indirectly from an abnormal activity or expression level of CDK can be an intended disease or disorder condition. In some embodiments, the said method for treating a disease or disorder condition associated with CDK in a subject or a subject in need thereof comprises administering to the subject a compound or a pharmaceutically acceptable salt thereof as described herein.

Different disease or disorder conditions associated with CDK have been reported. CDK has been implicated, for example, auto-immune diseases, neurodegeneration (such as Parkinson's disease, Alzheimer's disease, and ischemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangio sarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangio sarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer. In some embodiments, the said method comprises administering to a subject or a subject in need thereof, a compound or a pharmaceutically acceptable salt thereof as described herein.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

In some embodiments, are provided Compounds as described herein in use for treating a disease or disorder associated with aberrant CDK activity in a subject or a subject in need thereof, wherein the use comprises administering to the subject an effective amount of one or more compounds as described herein, pharmaceutically acceptable salts, solvates, pharmaceutical compositions, or prodrugs thereof.

In some embodiments, are provided pharmaceutical compositions as described herein in use for treating a disease or disorder associated with aberrant CDK activity in a subject or a subject in need thereof, wherein the use comprises administering to the subject an effective amount of one or more pharmaceutical compositions as described herein.

In some embodiments, are provided uses of compounds as described herein in the manufacture of a formulation treating a disease or disorder associated with aberrant CDK activity in a subject or a subject in need thereof, wherein the use comprises administering to the subject an effective amount of one or more compounds as described herein, pharmaceutically acceptable salts, solvates, pharmaceutical compositions, or prodrugs thereof.

In some embodiments, use of pharmaceutical compositions as described herein for treating a disease or disorder associated with aberrant CDK activity in a subject or a subject in need thereof, wherein the use comprises administering to the subject an effective amount of one or more pharmaceutical composition as described herein. In some embodiments, the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

In some embodiments, are provided compounds as described herein in use for treating cancer in a subject or a subject in need thereof, wherein the use comprises administering to the subject an effective amount of one or more compounds as described herein, pharmaceutically acceptable salts, solvates, pharmaceutical compositions, or prodrugs thereof.

In some embodiments, are provided pharmaceutical compositions as described herein in use for treating cancer in a subject or a subject in need thereof, wherein the use comprises administering to the subject an effective amount of one or more pharmaceutical compositions as described herein.

In some embodiments, are provided uses of compounds as described herein in the manufacture of a formulation treating cancer in a subject or a subject in need thereof, wherein the use comprises administering to the subject an effective amount of one or more compounds as described herein, pharmaceutically acceptable salts, solvates, pharmaceutical compositions, or prodrugs thereof.

In some embodiments, are provided uses of pharmaceutical compositions as described herein for treating cancer in a subject or a subject in need thereof, wherein the use comprises administering to the subject an effective amount of one or more pharmaceutical compositions as described herein. In some embodiments, the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising thereof, can be administered in combination with agonists of nuclear receptors agents.

In other methods, the compounds of the disclosure, as well as pharmaceutical compositions comprising thereof, can be administered in combination with antagonists of nuclear receptors agents.

In other methods, the compounds of the disclosure, as well as pharmaceutical compositions comprising thereof, can be administered in combination with an anti-proliferative agent.

### Combination Therapies

For treating cancer and other proliferative diseases, the compounds can be used in combination with chemotherapeutic agents, agonists or antagonists of nuclear receptors, or other anti-proliferative agents. The compounds can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound can be administered in combination with a corticosteroid such as triamcinolone, dexamethasone, fluocinolone, cortisone, prednisolone, or flumetholone.

For treating autoimmune or inflammatory conditions, the compound can be administered in combination with an immune suppressant such as fluocinolone acetonide (Retisert®), rimexolone (AL-2178, Vexol, Alcon), or cyclosporine (Restasis®).

In some embodiments, the compounds are used in methods of prevention (prevent or preventing) or prophalyxis of the diseases, disorders, or conditions provided herein. In some embodiments, the compounds are used to prevent the recurrence of a condition or disease provided herein.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner.

In some embodiments, the following embodiments are provided:
1. A compound having Formula (I) or a pharmaceutically acceptable salt or solvate thereof:

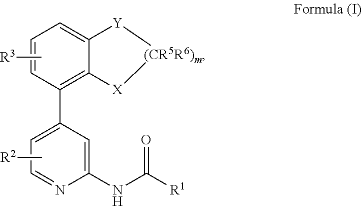

wherein
X is O, S, or CR⁷R⁸;
Y is O, S, CR⁹R¹⁰, or NR⁴;
m is 1-3;
R² and R³ is selected from H, D, halogen, oxo, CN, C₁₋₃ alkyl, C₁₋₃ alkoxy, C₂₋₄ alkenyl, C₂₋₄ alkynyl, C₁₋₃ haloalkyl, and C₁₋₃ haloalkoxy;
R⁴ is selected from H, C₁₋₃ alkyl, C₁₋₃ alkoxy, C₁₋₃ haloalkyl, and —C(O) C₁₋₃ alkyl;
R¹ is selected from C₁₋₆ alkyl, C₁₋₆ alkoxy, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, and (4-14 membered heterocycloalkyl)-C₁₋₄ alkyl;
wherein R¹ is optionally substituted with 1, 2, 3, 4, 5, 6, 7 or 8 independently selected R^{b} substituents;
each R⁵, R⁶, R⁷, R⁸, R⁹, and R¹⁰ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1}, S(O)₂R^{a1}, SF₅, P(O)R^{a1}R^{a1}, P(O)(OR^{a1})(OR^{a1}), B(OR^{a1})₂, and S(O)₂NR^{a1}R^{a1};
wherein when R², R³, R⁴, R⁵, R⁶, R⁷, R⁸, R⁹, or R¹⁰ is C₁₋₆ alkyl, C₁₋₆ alkoxy, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, or (4-14 membered heterocycloalkyl)-C₁₋₄ alkyl, then R², R³, R⁴, R⁵, R⁶, R⁷, R⁸, R⁹, and R¹⁰ is optionally substituted with 1, 2, 3, 4 or 5 independently selected R^{b} substituents;
optionally R⁵ and R⁶ together with the carbon atom to which they are both attached form a C₄₋₇ spirocyclic ring optionally substituted with 1, 2, 3, 4 or 5 independently selected R^{b} substituents;
optionally R⁴ and R⁵ together with the atoms to which they are attached form a 4-, 5-, 6-, or 7-membered heterocycloalkyl ring optionally substituted with 1, 2, 3, 4 or 5 independently selected R^{b} substituents;
optionally R⁴ and R⁶ together with the atoms to which they are attached form a 4-, 5-, 6-, or 7-membered heterocycloalkyl ring optionally substituted with 1, 2, 3, 4 or 5 independently selected R^{b} substituents;
optionally R⁷ and R⁸ together with the carbon atom to which they are both attached form a C₃-C₇ spirocyclic ring optionally substituted with 1, 2, 3, 4 or 5 independently selected R^{b} substituents,
optionally one of R⁵ and R⁶ and one of R⁷ and R⁸ together with the atoms to which they are attached form a 4-, 5-, 6-, or 7-membered cycloalkyl ring optionally substituted with 1, 2, 3, 4 or 5 independently selected R^{b} substituents; and
optionally one of R⁵ and R⁶ and one of R⁹ and R¹⁰ together with the atoms to which they are attached form a 4-, 5-, 6-, or 7-membered cycloalkyl ring optionally substituted with 1, 2, 3, 4 or 5 independently selected R^{b} substituents; and
wherein when m is 2 or 3, then two R⁵ or two R⁶ together with the atoms to which they are attached optionally form a 4-, 5-, 6-, or 7-membered cycloalkyl or heterocycloalkyl ring optionally substituted with 1, 2, 3, 4 or 5 independently selected R^{b} substituents;
each R^{a1} is independently selected from H, D, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄alkyl, C₃₋₁₀ cycloalkyl-C₁₋₄ alkyl, (5-10 membered heteroaryl)-C₁₋₄ alkyl, and (4-14 membered heterocycloalkyl)-C₁₋₄ alkyl;
wherein when R^{a1} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a1} is optionally substituted with 1, 2, 3, 4, or 5 independently selected R^{d} substituents;
each R^{b} substituent is independently selected from D, halo, oxo, C₁₋₄ alkyl, C₁₋₆ alkoxy, C₁₋₄ haloalkyl, C₁₋₄ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, NR^{c}S(O)(NR^{c})R^{c}, S(O)NR^{c}R^{c}, S(O)₂R^{c}, and S(O)₂NR^{c}R^{c};
wherein when R^{b} is C₁₋₄ alkyl, C₁₋₆ alkoxy, C₁₋₄ haloalkyl, C₁₋₄ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{b} is optionally substituted with 1, 2, or 3 independently selected R^{d} substituents;
each R^{c} is independently selected from H, D, —OH, C₁₋₆ alkyl, C₁₋₆ alkoxy, C₁₋₄ haloalkyl, C₂₋₆alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl;
wherein when R^{c} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents;
each R^{f} is independently selected from halogen, C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R^{g}, P(O)(OR^{g})(OR^{g}), S(O)R^{g}, NR^{g}S(O)(NR^{g})R^{g}, S(O)NR^{g}R^{g}, S(O)₂R^{g}, NR^{g}S(O)₂R^{g}, NR^{g} S(O)₂NR^{g}R^{g}, and S(O)₂NR^{g}R^{g};
wherein when R^{f} is C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents;
each Rⁿ is independently selected from C₁₋₄ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, NR^{o}S(O)(NR^{o})R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{g}, C(O)R^{c}, C(O)NR^{e}R^{e}, C(O)OR^{c}, OC(O)R^{c}, OC(O)NR^{e}R^{e}, NHR^{e}, NR^{e}R^{e}, NR^{c}C(O)R^{c}, NR^{c}C(O)NR^{c}R^{c}, NR^{e}C(O)OR^{e}, C(═NR^{e})NR^{e}R^{e}, NR^{e}C(═NR^{e})NR^{e}R^{e}, NR^{c}C(═NOH)NR^{e}R^{e}, NR^{c}C(═NCN)NR^{e}R^{e}, SF₅, P(O)R^{c}R^{c}, P(O)(OR^{c})(OR^{c}), S(O)R^{c}, NR^{c}S(O)(NR^{a1})R^{c}, S(O)NR^{c}R^{c}, S(O)₂R^{c}, NR^{c}S(O)₂R^{c}, NR^{e}S(O)₂NR^{e}R^{e}, and S(O)₂NR^{e}R^{e},
wherein when R^{d} is C₁₋₆ alkyl, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents;
each R^{e} is independently selected from H, D, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{e} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents;
each R^{g} is independently selected from H, D, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl,
wherein when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents;
each R^{P} is independently selected from C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{c}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, NR^{r}S(O)(NR^{r})R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r};
each R^{o} or R^{c} is independently selected from H, D, C₁₋₄ alkyl, C₃₋₆ cycloalkyl, C₆₋₁₀ aryl, 5 or 6-membered heteroaryl, C₁₋₄ haloalkyl, C₂₋₄ alkenyl, and C₂₋₄ alkynyl,
wherein when R^{o} or R^{r} is C₁₋₄ alkyl, C₃₋₆ cycloalkyl, C₆₋₁₀ aryl, 5 or 6-membered heteroaryl, C₂₋₄ alkenyl, and C₂₋₄ alkynyl, then R^{o} or R^{r} is optionally substituted with 1, 2 or 3 independently selected R^{q} substituents;
each R^{q} is independently selected from D, OH, CN, —COOH, NH₂, halo, C₁₋₆ alkyl, C₁₋₆ haloalkyl, C₁₋₆ alkoxy, C₁₋₆ haloalkoxy, C₁₋₄ alkylthio, phenyl, 5-6 membered heteroaryl, C₃₋₆ cycloalkyl, 4-6 membered heterocycloalkyl, —CONHR¹¹, —NHC(O)R¹¹, —OC(O)R¹¹, C(O)OR¹¹, —C(O)R¹¹, —SO₂R¹¹, —NHSO₂R¹¹, —SO₂NHR¹¹ and NR¹¹R¹¹,
wherein when R^{q} is C₁₋₆ alkyl, phenyl, 4-6 membered heterocycloalkyl or 5-6 membered heteroaryl, then R^{q} is optionally substituted with OH, CN, —COOH, NH₂, C₁₋₆ alkoxy, C₃₋₆cycloalkyl or 4-6 membered heterocycloalkyl; and
   each R¹¹ is independently C₁₋₆ alkyl.
2. The compound of embodiment 1, wherein the compound has a formula of

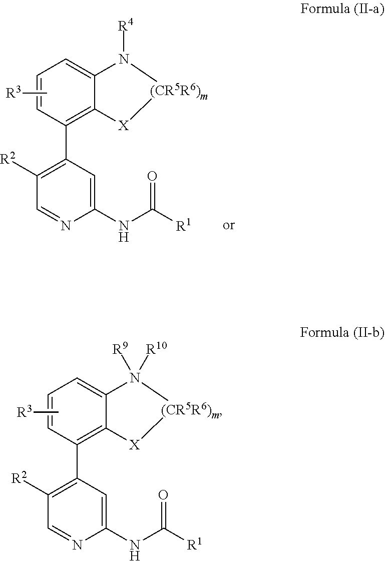

or a pharmaceutically acceptable salt or solvate thereof, wherein the variables are as defined in embodiment 1.
3. The compound of embodiment 2, or a pharmaceutically acceptable salt or solvate thereof, wherein X is O.
4. The compound of embodiment 3, or a pharmaceutically acceptable salt or solvate thereof, wherein m is 2.
5. The compound of embodiment 4, or a pharmaceutically acceptable salt or solvate thereof, wherein the compound has a formula of

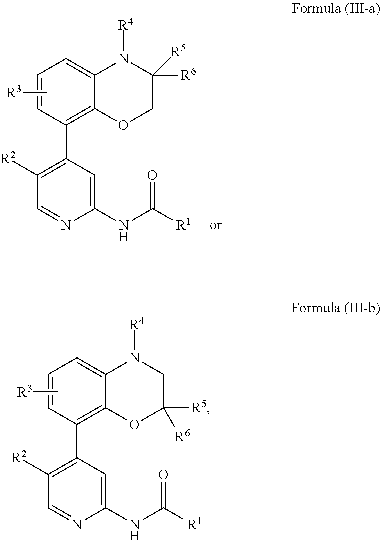

wherein the variables are as defined in embodiment 1.
6. The compound of embodiment 5, or a pharmaceutically acceptable salt or solvate thereof, wherein R⁵ and R⁶ are independently H or C₁₋₆ alkyl.
7. The compound of embodiment 5, or a pharmaceutically acceptable salt or solvate thereof, wherein R⁵ and R⁶ are independently C₁₋₆ alkyl.
8. The compound of embodiment 5, or a pharmaceutically acceptable salt or solvate thereof, wherein both R⁵ and R⁶ are CH₃.
9. The compound of embodiment 5, or a pharmaceutically acceptable salt or solvate thereof, wherein R⁵ is H.
10. The compound of embodiment 9, or a pharmaceutically acceptable salt or solvate thereof, wherein the compound has a formula of

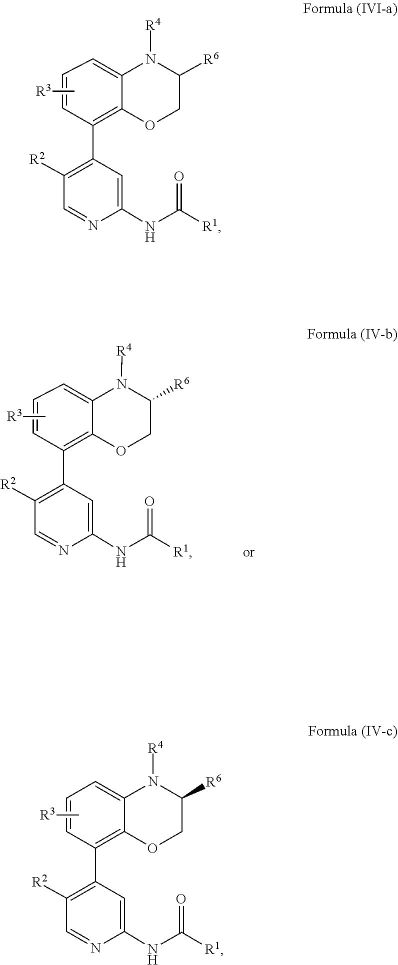

wherein the variables as defined in embodiment 1.
11. The compound of embodiment 10, or a pharmaceutically acceptable salt or solvate thereof, wherein R⁶ is H or C₁₋₆ alkyl.
12. The compound of any one of embodiments 10-11, or a pharmaceutically acceptable salt or solvate thereof, wherein R⁶ is C₁₋₆ alkyl.
13. The compound of any one of embodiments 10-12, or a pharmaceutically acceptable salt or solvate thereof, wherein R⁶ is CH₃.
14. The compound of embodiment 4, or a pharmaceutically acceptable salt or solvate thereof, wherein the compound has a formula of

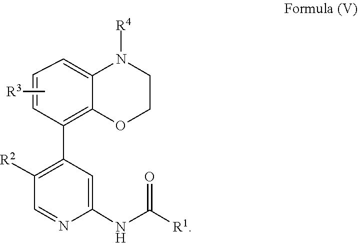

15. The compound of any one of embodiments 1-14, or a pharmaceutically acceptable salt or solvate thereof, wherein R¹ is C₃₋₁₀ cycloalkyl optionally substituted with 1, 2, 3, or 4 independently selected R^{b} substituents.
16. The compound of any one of embodiments 1-15, or a pharmaceutically acceptable salt or solvate thereof, wherein R¹ is C₃₋₇ cycloalkyl optionally substituted with 1, 2, 3, or 4 independently selected R^{b} substituents.
17. The compound of any one of embodiments 1-16, or a pharmaceutically acceptable salt or solvate thereof, wherein R¹ is C₅₋₆ cycloalkyl optionally substituted with 1, 2, 3, or 4 independently selected R^{b} substituents.
18. The compound of embodiment 17, or a pharmaceutically acceptable salt or solvate thereof, wherein R¹ is

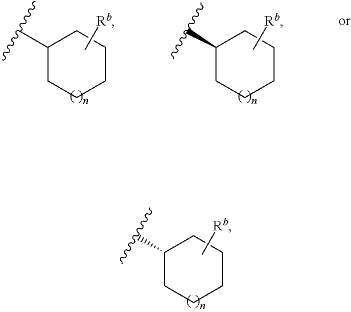

wherein n is 0 or 1 and R^{b} is as defined in embodiment 1.
19. The compound of any one of embodiments 1-18, or a pharmaceutically acceptable salt or solvate thereof, wherein R¹ is

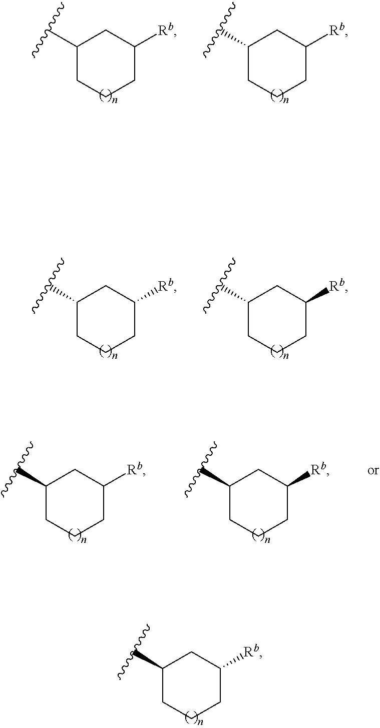

wherein n is 0 or 1 and R^{b} is as defined in embodiments 1-9.
20. The compound of any one of embodiments 18-19, or a pharmaceutically acceptable salt or solvate thereof, wherein R^{b} is NR^{c}C(O)R^{c}, NR^{c}C(O)NR^{c}R^{c}, NR^{c}S(O)(NR^{c})R^{c}, or NR^{c}S(O)₂NR^{c}R^{c}.
21. The compound of embodiment 20, or a pharmaceutically acceptable salt or solvate thereof, wherein the R^{c} in NR^{c}C(O)R^{c}, NR^{c}C(O)NR^{c}R^{c}, or NR^{c}S(O)₂NR^{c}R^{c} is independently H, C₁₋₆ alkyl, C₃₋₁₀ cycloalkyl, 4-10 membered heterocycloalkyl, or (5-10 membered heteroaryl)-C₁₋₄ alkyl; wherein when R^{e} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, then W is optionally substituted with 1, 2, 3, 4, or 5 independently selected R^{f} substituents.
22. The compound of embodiment 21, or a pharmaceutically acceptable salt or solvate thereof, wherein the R^{f} substituents are independently halogen, CN or OR^{g}.
23. The compound of embodiment 22, or a pharmaceutically acceptable salt or solvate thereof, wherein the R^{g} is independently H or C₁₋₆ alkyl.
24. The compound of any one of embodiments 1-23, or a pharmaceutically acceptable salt or solvate thereof, wherein R^{b} is acetamido (—NHC(O)CH₃), 3-hydroxybutanamido (—NHC(O)CH₂CH(OH)CH₃), propionamido (—NHC(O)CH₂CH₃), 2-methoxyacetamido (—NHC(O)CH₂—OCH₃), 2-cyanoacetamido (—NHC(O)CH₂—CN), 1-hydroxycyclopropane-1-carboxamido,

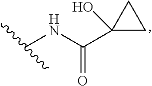 [SMILES: *NC(=O)C1(O)CC1]

2-(thiazol-4-yl)acetamido,

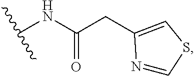 [SMILES: *NC(=O)Cc1cscn1]

methylsulfonamido (—NSO₂CH₃), 3-methylureido (—NC(O)NHCH₃), 3-methoxyureido (—NC(O)NHOCH₃), 3,3-dimethylureido (—NC(O)N(CH₃)₂), or 3-ethylureido (—NC(O)NHCH₂CH₃), morpholine-4-carboxamido, i.e.,

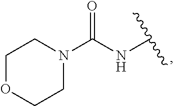 [SMILES: *NC(=O)N1CCOCC1]

or 4-methylpiperazine-1-carboxamide, i.e.,

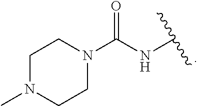 [SMILES: *NC(=O)N1CCN(C)CC1]

25. The compound of embodiment 24, or a pharmaceutically acceptable salt or solvate thereof, wherein the compound has a formula of

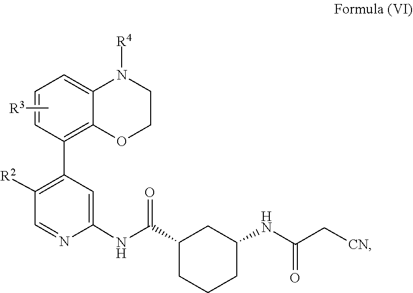

wherein the variables are as defined in embodiment 1.
26. The compound of embodiment 25, or a pharmaceutically acceptable salt or solvate thereof, wherein the compound has a formula of

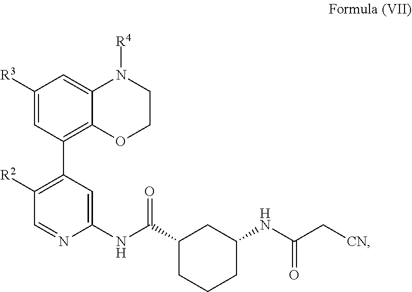

wherein the variables are as defined in embodiment 1.
27. The compound of any one of embodiments 1-26, or a pharmaceutically acceptable salt or solvate thereof, wherein R² is H, halogen, or C₁₋₆ alkyl.
28. The compound of any one of embodiments 1-27, or a pharmaceutically acceptable salt or solvate thereof, wherein R² is halogen.
29. The compound of any one of embodiments 1-28, or a pharmaceutically acceptable salt or solvate thereof, wherein R² is Cl.
30. The compound of any one of embodiments 1-27, or a pharmaceutically acceptable salt or solvate thereof, wherein R² is C₁₋₆ alkyl.
31. The compound of any one of embodiments 1-27, or a pharmaceutically acceptable salt or solvate thereof, wherein R² is CH₃.
32. The compound of any one of embodiments 1-31, or a pharmaceutically acceptable salt or solvate thereof, wherein R³ is H or halogen.
33. The compound of any one of embodiments 1-32, or a pharmaceutically acceptable salt or solvate thereof, wherein R³ is halogen.
34. The compound of any one of embodiments 1-33, or a pharmaceutically acceptable salt or solvate thereof, wherein R³ is F.
35. The compound of embodiment 34, or a pharmaceutically acceptable salt or solvate thereof, wherein the compound have a formula of

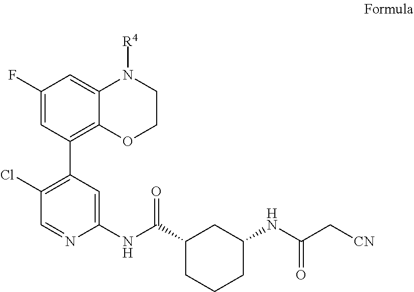

wherein R⁴ is as defined in embodiment 1.
36. The compound of embodiment 35, or a pharmaceutically acceptable salt or solvate thereof, wherein R⁴ is H, C₁₋₆ alkyl, or C₁₋₆alkoxy.
36. The compound of embodiment 35, or a pharmaceutically acceptable salt or solvate thereof, wherein R⁴ is H.
37. The compound of embodiment 3, or a pharmaceutically acceptable salt or solvate thereof, wherein the compound has a formula of

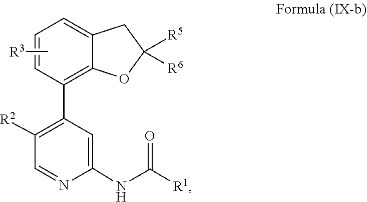

wherein the variables are as defined in embodiment 1.
38. The compound of embodiment 37, or a pharmaceutically acceptable salt or solvate thereof, wherein R⁵ and R⁶ are independently H or C₁₋₆ alkyl.
39. The compound of embodiment 37, or a pharmaceutically acceptable salt or solvate thereof, wherein both R⁵ and R⁶ are C₁₋₆ alkyl.
40. The compound of embodiment 37, or a pharmaceutically acceptable salt or solvate thereof, wherein both R⁵ and R⁶ are CH₃.
41. The compound of embodiment 1, or a pharmaceutically acceptable salt or solvate thereof, wherein the compound has a formula of

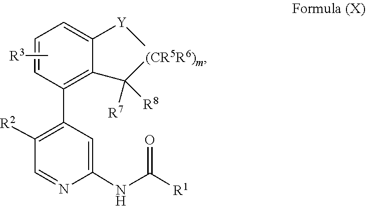

wherein the variables are as defined in embodiment 1.
42. The compound of embodiment 41, or a pharmaceutically acceptable salt or solvate thereof, wherein the compound has a formula of

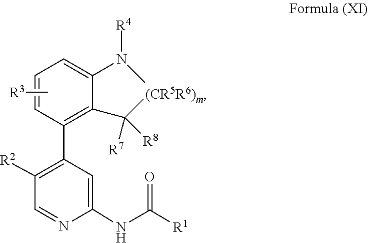

wherein the variables are as defined in embodiment 1.
43. The compound of embodiment 42, or a pharmaceutically acceptable salt or solvate thereof, wherein m is 2.
44. The compound of embodiment 43, or a pharmaceutically acceptable salt or solvate thereof, wherein the compound has a formula of

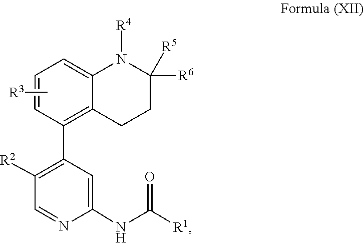

wherein the variables are as defined in embodiment 1.
45 The compound of embodiment 44, or a pharmaceutically acceptable salt or solvate thereof, wherein R⁵ and R⁶ are independently H or C₁₋₆ alkyl.
46. The compound of embodiment 44, or a pharmaceutically acceptable salt or solvate thereof, wherein both R⁵ and R⁶ are H.
47. The compound of embodiment 41, or a pharmaceutically acceptable salt or solvate thereof, wherein the compound has a formula of

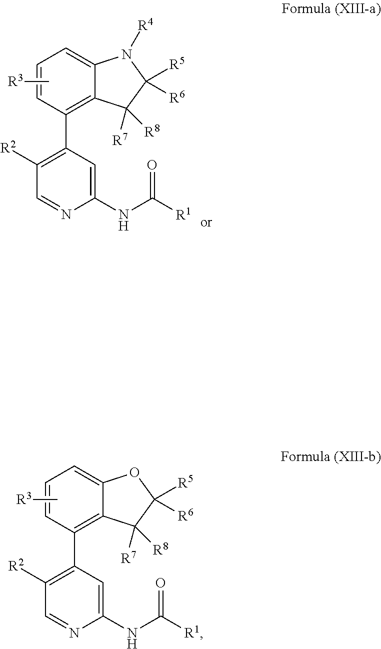

wherein the variables are as defined in embodiment 1.
48. The compound of embodiment 43, or a pharmaceutically acceptable salt or solvate thereof, wherein R⁵, R⁶, R⁷, and R⁸ are independently H or C₁₋₆ alkyl.
49. The compound of embodiment 48, or a pharmaceutically acceptable salt or solvate thereof, wherein both R⁷ and R⁸ are H.
50. The compound of embodiment 49, or a pharmaceutically acceptable salt or solvate thereof, wherein R⁵ is H.
51. The compound of embodiment 50, or a pharmaceutically acceptable salt or solvate thereof, wherein both R⁵ and R⁶ are C₁₋₆ alkyl.
52. The compound of embodiment 51, or a pharmaceutically acceptable salt or solvate thereof, wherein both R⁵ and R⁶ are CH₃.
53. The compound of any one of embodiments 37-52, or a pharmaceutically acceptable salt or solvate thereof, wherein R¹ is C₃₋₁₀cycloalkyl optionally substituted with 1, 2, 3, or 4 independently selected R^{b} substituents.
54. The compound of any one of embodiments 37-53, or a pharmaceutically acceptable salt or solvate thereof, wherein R¹ is C₃₋₇ cycloalkyl optionally substituted with 1, 2, 3, or 4 independently selected R^{b} substituents.
55. The compound of any one of embodiments 37-54, or a pharmaceutically acceptable salt or solvate thereof, wherein R¹ is C₅₋₆ cycloalkyl optionally substituted with 1, 2, 3, or 4 independently selected R^{b} substituents.
56. The compound of embodiment 55, or a pharmaceutically acceptable salt or solvate thereof, wherein R¹ is

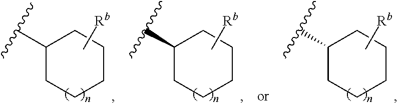

wherein n is 0 or 1 and R^{b} is as defined in embodiment 1.
57. The compound of any one of embodiments 37-56, or a pharmaceutically acceptable salt or solvate thereof, wherein R¹ is

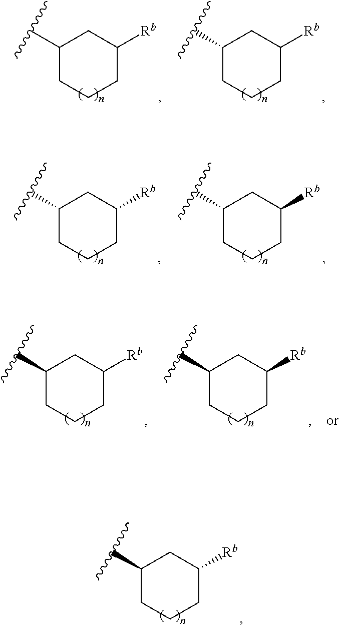

wherein n is 0 or 1 and R^{b} is as defined in embodiments 1-9.
58. The compound of any one of embodiments 56-57, or a pharmaceutically acceptable salt or solvate thereof, wherein R^{b} is NR^{c}C(O)R^{c}, NR^{c}C(O)NR^{c}R^{c}, NR^{c}S(O)(NR^{c})R^{c}, or NR^{c}S(O)₂NR^{c}R^{c}.
59. The compound of embodiment 58, or a pharmaceutically acceptable salt or solvate thereof, wherein the R^{c} in NR^{c}C(O)R^{c}, NR^{c}C(O)NR^{c}R^{c}, NR^{c}S(O)(NR^{c})R^{c}, or NR^{c}S(O)₂NR^{c}R^{c} is independently H, C₁₋₆ alkyl, C₃₋₁₀ cycloalkyl, 4-10 membered heterocycloalkyl, or (5-10 membered heteroaryl)-C₁₋₄ alkyl; wherein when R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, then R^{c} is optionally substituted with 1, 2, 3, 4, or 5 independently selected R^{f} substituents.
60. The compound of embodiment 59, or a pharmaceutically acceptable salt or solvate thereof, wherein the R^{f} substituents are independently halogen, CN or OR^{g}.
61. The compound of embodiment 60, or a pharmaceutically acceptable salt or solvate thereof, wherein the R^{g} is independently H or C₁₋₆ alkyl.
62. The compound of any one of embodiments 37-61, or a pharmaceutically acceptable salt or solvate thereof, wherein R^{b} is acetamido (—NHC(O)CH₃), 3-hydroxybutanamido (—NHC(O)CH₂CH(OH)CH₃), propionamido (—NHC(O)CH₂CH₃), 2-methoxyacetamido (—NHC(O)CH₂—OCH₃), 2-cyanoacetamido (—NHC(O)CH₂—CN), 1-hydroxycyclopropane-1-carboxamido,

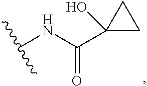 [SMILES: *NC(=O)C1(O)CC1]

2-(thiazol-4-yl)acetamido,

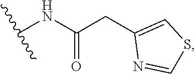 [SMILES: *NC(=O)Cc1cscn1]

methylsulfonamido (—NSO₂CH₃), 3-methylureido (—NC(O)NHCH₃), 3-methoxyureido (—NC(O)NHOCH₃), 3,3-dimethylureido (—NC(O)N(CH₃)₂), or 3-ethylureido (—NC(O)NHCH₂CH₃), morpholine-4-carboxamido, i.e.,

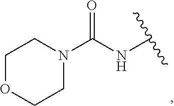 [SMILES: *NC(=O)N1CCOCC1]

or 4-methylpiperazine-1-carboxamide, i.e.,

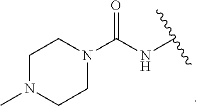 [SMILES: *NC(=O)N1CCN(C)CC1]

63. The compound of any one of embodiments 37-62, or a pharmaceutically acceptable salt or solvate thereof, wherein R² is H, halogen, or C₁₋₆ alkyl.
64. The compound of any one of embodiments 37-62, or a pharmaceutically acceptable salt or solvate thereof, wherein R² is halogen.
65. The compound of any one of embodiments 37-62, or a pharmaceutically acceptable salt or solvate thereof, wherein R² is Cl.
66. The compound of any one of embodiments 37-65, or a pharmaceutically acceptable salt or solvate thereof, wherein R³ is H, halogen, or C₁₋₆ alkyl
67. The compound of any one of embodiments 37-66, or a pharmaceutically acceptable salt or solvate thereof, wherein R³ is H
68. The compound of any one of embodiments 37-67, or a pharmaceutically acceptable salt or solvate thereof, wherein R⁴ is H.
69. The compound of embodiment J wherein die compound is selected from the group consisting of:
(1S,3R)-3-acetamido-N-(5-chloro-4-(indolin-4-yl)pyridin-2-yl)cyclohexane-1-carboxamide;
(1S,3R)-3-acetamido-N-(5-chloro-4-(2-methylindolin-4-yl)pyridin-2-yl)cyclohexane-1-carboxamide;
(1S,3R)-3-acetamido-N-(5-chloro-4-(1,2,3,4-tetrahydroquinolin-5-yl)pyridin-2-yl)cyclohexane-1-carboxamide;
(1S,3R)-3-acetamido-N-(5-chloro-4-(3,4-dihydro-2H-benzo[b][1,4]oxazin-8-yl)pyridin-2-yl)cyclohexane-1-carboxamide;
(1S,3R)-3-acetamido-N-(5-chloro-4-(2,2-dimethylindolin-4-yl)pyridin-2-yl)cyclohexane-1-carboxamide;
(1S,3R)-3-(2-cyanoacetamido)-N-(4-(2,2-dimethylindolin-4-yl)-5-methylpyridin-2-yl)cyclohexane-1-carboxamide;
(1S,3R)-3-(2-cyanoacetamido)-N-(4-(3,3-dimethyl-3,4-dihydro-2H-benzo[b][1,4]oxazin-8-yl)-5-methylpyridin-2-yl)cyclohexane-1-carboxamide;
(1S,3R)-3-(2-cyanoacetamido)-N-(4-(3,4-dihydro-2H-benzo[b][1,4]oxazin-8-yl)-5-methylpyridin-2-yl)cyclohexane-1-carboxamide;
(1S,3R)-3-acetamido-N-(5-chloro-4-(3,3-dimethyl-3,4-dihydro-2H-benzo[b][1,4]oxazin-8-yl)pyridin-2-yl)cyclohexane-1-carboxamide;
(1S,3R)—N-(5-chloro-4-(3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(3,3-dimethyl-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3-methyl-3,4-dihydro-2H-benzo[b][1,4]oxazin-8-yl)pyridin-2-yl)-3-(2-cyanoacetamido)cyclohexane-1-carboxamide;
(1S,3R)-3-acetamido-N-(5-chloro-4-(2,2-dimethyl-2,3-dihydrobenzofuran-4-yl)pyridin-2-yl)cyclohexane-1-carboxamide; and
(1S,3R)—N-(5-chloro-4-(2,2-dimethyl-2,3-dihydrobenzofuran-7-yl)pyridin-2-yl)-3-(2-cyanoacetamido)cyclohexane-1-carboxamide.
   70. A pharmaceutical composition comprising a compound according to any one of embodiments 1-69, or a pharmaceutically acceptable salt or solvate thereof, and optionally a pharmaceutically acceptable excipient.
   71. The pharmaceutical composition of embodiment 70, wherein the pharmaceutical composition comprises an enantiomeric excess of at least 90% of one enantiomer of the compound, or a pharmaceutically acceptable salt or solvate thereof.
   72. The pharmaceutical composition of embodiment 70, wherein the pharmaceutical composition comprises an enantiomeric excess of at least 95% of one enantiomer of the compound, or a pharmaceutically acceptable salt or solvate thereof.
   73. The pharmaceutical composition of embodiment 70, wherein the pharmaceutical composition comprises an enantiomeric excess of at least 98% of one enantiomer of the compound, or a pharmaceutically acceptable salt or solvate thereof.
   74. The pharmaceutical composition of embodiment 70, wherein the pharmaceutical composition comprises an enantiomeric excess of at least 99% of one enantiomer of the compound, or a pharmaceutically acceptable salt or solvate thereof.
   75. A method of inhibiting a CDK enzyme comprising: contacting the CDK enzyme with an effective amount of a compound of any one of embodiments 1-69, or a pharmaceutically acceptable salt or solvate thereof, or a pharmaceutical composition of any one of embodiments 70-74.
   76. The method of embodiment 75, wherein the CDK enzyme is CDK9.
   77. A method of treating a disease or disorder associated with aberrant CDK activity in a subject or a subject in need thereof comprising administering to the subject, a compound of any one of embodiments 1-691-69, or a pharmaceutically acceptable salt or solvate thereof, or a pharmaceutical composition of any one of embodiments 70-74.
   78. The method of embodiment 77, wherein the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
   79. A method of treating cancer in a subject or a subject in need thereof comprising administering to the subject, a compound of any one of embodiments 1-69, or a pharmaceutically acceptable salt or solvate thereof.
   80. The method of embodiment 79, wherein the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
   81. A compound of any one of embodiments 1-69 in use for inhibiting a CDK enzyme in a subject, wherein the use comprises administering to the subject an effective amount of the compound of any one of embodiments 1-69, or a pharmaceutically acceptable salt, a solvate, a pharmaceutical composition, or a prodrug thereof.
   82. A pharmaceutical composition of any one of embodiments 70-74 in use for inhibiting a CDK enzyme in a subject, wherein the use comprises administering to the subject an effective amount of the pharmaceutical composition of any one of embodiments 70-74.
   83. Use of a compound of any one of embodiments 1-69 in the manufacture of a formulation inhibiting a CDK enzyme in a subject, wherein the use comprises administering to the subject an effective amount of the compound of any one of embodiments 1-69, or a pharmaceutically acceptable salt, a solvate, a pharmaceutical composition, or a prodrug thereof.
   84. Use of a pharmaceutical composition of any one of embodiments 70-74 for inhibiting a CDK enzyme in a subject, wherein the use comprises administering to the subject an effective amount of the pharmaceutical composition of any one of embodiments 70-74.
   85. The use of any one of embodiments 81-84, wherein the CDK enzyme is CDK9.
   86. A compound of any one of embodiments 1-69 in use for treating a disease or disorder associated with aberrant CDK activity in a subject or a subject in need thereof, wherein the use comprises administering to the subject an effective amount of the compound of any one of embodiments 1-69, or a pharmaceutically acceptable salt, a solvate, a pharmaceutical composition, or a prodrug thereof.
   87. A pharmaceutical composition of any one of embodiments 70-74 in use for treating a disease or disorder associated with aberrant CDK activity in a subject or a subject in need thereof, wherein the use comprises administering to the subject an effective amount of the pharmaceutical composition of any one of embodiments 70-74.
   88. Use of a compound of any one of embodiments 1-69 in the manufacture of a formulation treating a disease or disorder associated with aberrant CDK activity in a subject or a subject in need thereof, wherein the use comprises administering to the subject an effective amount of the compound of any one of embodiments 1-69, or a pharmaceutically acceptable salt, a solvate, a pharmaceutical composition, or a prodrug thereof.
   89. Use of a pharmaceutical composition of any one of embodiments 70-74 for treating a disease or disorder associated with aberrant CDK activity in a subject or a subject in need thereof, wherein the use comprises administering to the subject an effective amount of the pharmaceutical composition of any one of embodiments 70-74.
   90. The use of any one of embodiments 86-89, wherein the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
   91. A compound of any one of embodiments 1-69 in use for treating cancer in a subject or a subject in need thereof, wherein the use comprises administering to the subject an effective amount of the compound of any one of embodiments 1-69, or a pharmaceutically acceptable salt, a solvate, a pharmaceutical composition, or a prodrug thereof.
   92. A pharmaceutical composition of any one of embodiments 70-74 in use for treating cancer in a subject or a subject in need thereof, wherein the use comprises administering to the subject an effective amount of the pharmaceutical composition of any one of embodiments 70-74.
   93. Use of a compound of any one of embodiments 1-69 in the manufacture of a formulation treating cancer in a subject or a subject in need thereof, wherein the use comprises administering to the subject an effective amount of the compound of any one of embodiments 1-69, or a pharmaceutically acceptable salt, a solvate, a pharmaceutical composition, or a prodrug thereof.
   94. Use of a pharmaceutical composition of any one of embodiments 70-74 for treating cancer in a subject or a subject in need thereof, wherein the use comprises administering to the subject an effective amount of the pharmaceutical composition of any one of embodiments 70-74.
   95. The use of any one of embodiments 90-94, wherein the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Compounds provided for herein include, for example, Examples 1-15, which have been either exemplified or identified in Table A and Table B. In some embodiments, compounds described herein can be made in trifluoroacetice acid (“TFA”) salt forms but the TFA salt form is just a non-limiting example of salt form and the compounds can also be made in other salt forms. For example, Examples 1, 5, 6, and 7 are prepared in TFA salt forms.

TABLE A

| Ex- | | | Calcd. | Found |
|---|---|---|---|---|
| ample | Structure | Chemical Name | (M + H)⁺ | (M + H)⁺ |
| 1 | 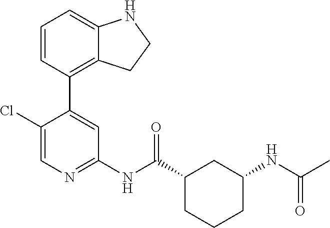 [SMILES: CC(=O)N[C@@H]1CCC[C@H](C(=O)Nc2cc(-c3cccc4c3CCN4)c(Cl)cn2)C1] | (1S,3R)-3-acetamido-N-(5- chloro-4-(indolin-4- yl)pyridin-2- yl)cyclohexane-1- carboxamide | 413.2 | 413.1 |
| 2 | 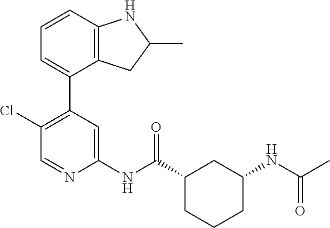 [SMILES: CC(=O)N[C@@H]1CCC[C@H](C(=O)Nc2cc(-c3cccc4c3CC(C)N4)c(Cl)cn2)C1] | (1S,3R)-3-acetamido-N-(5- chloro-4-(2-methylindolin- 4-yl)pyridin-2- yl)cyclohexane-1- carboxamide | 427.2 | 427.1 |
| 3 | 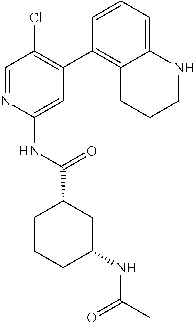 [SMILES: CC(=O)N[C@@H]1CCC[C@H](C(=O)Nc2cc(-c3cccc4c3CCCN4)c(Cl)cn2)C1] | (1S,3R)-3-acetamido-N-(5- chloro-4-(1,2,3,4- tetrahydroquinolin-5- yl)pyridin-2- yl)cyclohexane-1- carboxamide | 427.2 | 427.1 |
| 4 | 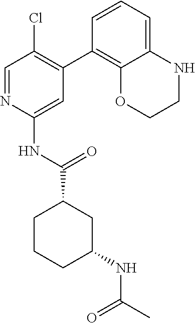 [SMILES: CC(=O)N[C@@H]1CCC[C@H](C(=O)Nc2cc(-c3cccc4c3OCCN4)c(Cl)cn2)C1] | (1S,3R)-3-acetamido-N-(5- chloro-4-(3,4-dihydro-2H- benzo[b][1,4]oxazin-8- yl)pyridin-2- yl)cyclohexane-1- carboxamide | 429.2 | 429.1 |
| 5 | 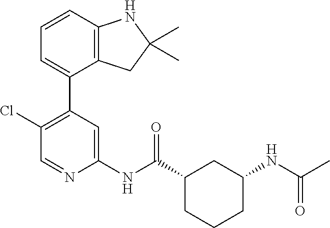 [SMILES: CC(=O)N[C@@H]1CCC[C@H](C(=O)Nc2cc(-c3cccc4c3CC(C)(C)N4)c(Cl)cn2)C1] | (1S,3R)-3-acetamido-N-(5- chloro-4-(2,2- dimethylindolin-4- yl)pyridin-2- yl)cyclohexane-1- carboxamide | 441.2 | 441.0 |
| 6 | 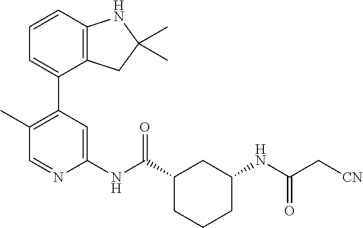 [SMILES: Cc1cnc(NC(=O)[C@H]2CCC[C@@H](NC(=O)CC#N)C2)cc1-c1cccc2c1CC(C)(C)N2] | (1S,3R)-3-(2- cyanoacetamido)-N-(4-(2,2- dimethylindolin-4-yl)-5- methylpyridin-2- yl)cyclohexane-1- carboxamide | 446.2 | 446.1 |
| 7 | 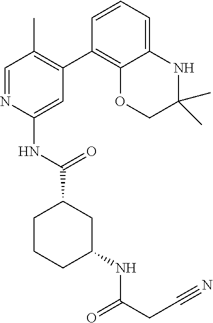 [SMILES: Cc1cnc(NC(=O)[C@H]2CCC[C@@H](NC(=O)CC#N)C2)cc1-c1cccc2c1OCC(C)(C)N2] | (1S,3R)-3-(2- cyanoacetamido)-N-(4-(3,3- dimethyl-3,4-dihydro-2H- benzo[b][1,4]oxazin-8-yl)- 5-methylpyridin-2- yl)cyclohexane-1- carboxamide | 462.2 | 462.1 |
| 8 | 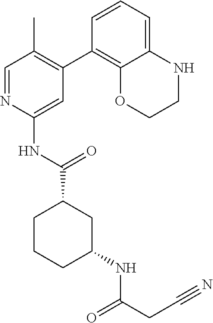 [SMILES: Cc1cnc(NC(=O)[C@H]2CCC[C@@H](NC(=O)CC#N)C2)cc1-c1cccc2c1OCCN2] | (1S,3R)-3-(2- cyanoacetamido)-N-(4-(3,4- dihydro-2H- benzo[b][1,4]oxazin-8-yl)- 5-methylpyridin-2- yl)cyclohexane-1- carboxamide | 434.2 | 434.1 |
| 9 | 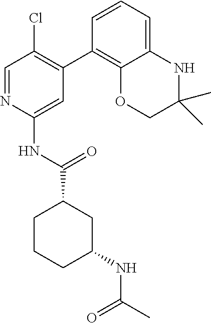 [SMILES: CC(=O)N[C@@H]1CCC[C@H](C(=O)Nc2cc(-c3cccc4c3OCC(C)(C)N4)c(Cl)cn2)C1] | (1S,3R)-3-acetamido-N-(5- chloro-4-(3,3-dimethyl-3,4- dihydro-2H- benzo[b][1,4]oxazin-8- yl)pyridin-2- yl)cyclohexane-1- carboxamide | 457.2 | 457.1 |
| 10 | 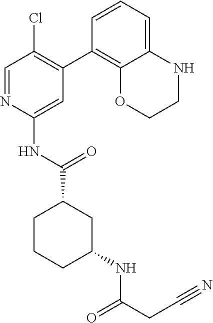 [SMILES: N#CCC(=O)N[C@@H]1CCC[C@H](C(=O)Nc2cc(-c3cccc4c3OCCN4)c(Cl)cn2)C1] | (1S,3R)-N-(5-chloro-4-(3,4- dihydro-2H- benzo[b][1,4]oxazin-8- yl)pyridin-2-yl)-3-(2- cyanoacetamido)cyclohexane- 1-carboxamide | 454.2/ 456.2 | 454.0/ 456.0 |
| 11 | 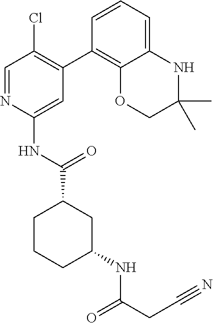 [SMILES: CC1(C)COc2c(cccc2-c2cc(NC(=O)[C@H]3CCC[C@@H](NC(=O)CC#N)C3)ncc2Cl)N1] | (1S,3R)-N-(5-chloro-4-(3,3- dimethyl-3,4-dihydro-2H- benzo[b][1,4]oxazin-8-yl) pyridin-2-yl)-3-(2- cyanoacetamido)cyclohexane- 1-carboxamide | 482.2 | 482.0 |
| 12 | 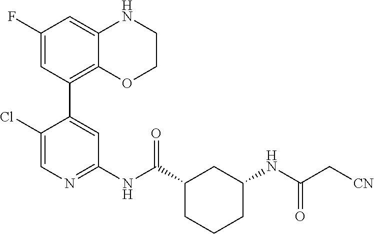 [SMILES: N#CCC(=O)N[C@@H]1CCC[C@H](C(=O)Nc2cc(-c3cc(F)cc4c3OCCN4)c(Cl)cn2)C1] | (1S,3R)-N-(5-chloro-4-(6- fluoro-3,4-dihydro-2H- benzo[b][1,4]oxazin-8- yl)pyridin-2-yl)-3-(2- cyanoacetamido)cyclohexane- 1-carboxamide | 472.2 | 472.2 |
| 13 | 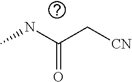 | (1S,3R)-N-(5-chloro-4-(6- fluoro-3-methyl-3,4- dihydro-2H- benzo[b][1,4]oxazin-8- yl)pyridin-2-yl)-3-(2- cyanoacetamido)cyclohexane- | 486.2 | 486.1 |
| | | 1-carboxamide | | |
| 14 | | (1S,3R)-3-acetamido-N-(5- | 442.2 | 442.2 |
| | | chloro-4-(2,2-dimethyl-2,3- | | |
| | | dihydrobenzofuran-4- | | |
| | | yl)pyridin-2- | | |
| | | yl)cyclohexane-1- | | |
| | | carboxamide | | |
| 15 | | (1S,3R)-N-(5-chloro-4-(2,2- | 467.2 | 467.1 |
| | | dimethyl-2,3- | | |
| | | dihydrobenzofuran-7- | | |
| | | yl)pyridin-2-yl)-3-(2- | | |
| | | cyanoacetamido)cyclohexane- | | |
| | | 1-carboxamide | | |

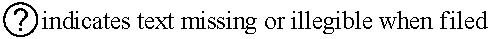

### Synthesis

Compounds of the disclosure, including salts thereof, can be prepared using known organic synthesis techniques and can be synthesized according to any of numerous possible synthetic routes.

The reactions for preparing compounds of the disclosure can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures, which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), or mass spectrometry, or by chromatography such as high performance liquid chromatography (“HPLC”) or thin layer chromatography.

The expressions, “ambient temperature,” “room temperature” “RT,” and “r.t.” as used herein, are understood in the art, and refer generally to a temperature, e.g. a reaction temperature, that is about the temperature of the room in which the reaction is carried out, for example, a temperature from about 20° C. to about 30° C.

Compounds of the disclosure can be prepared using numerous preparatory reactions known in the literature. The Schemes below provide general guidance in connection with preparing the compounds provided herein. One skilled in the art would understand that the preparations shown in the Schemes can be modified or optimized using general knowledge of organic chemistry to prepare various compounds. Example synthetic methods for preparing compounds are provided in the Schemes below.

### General Schemes

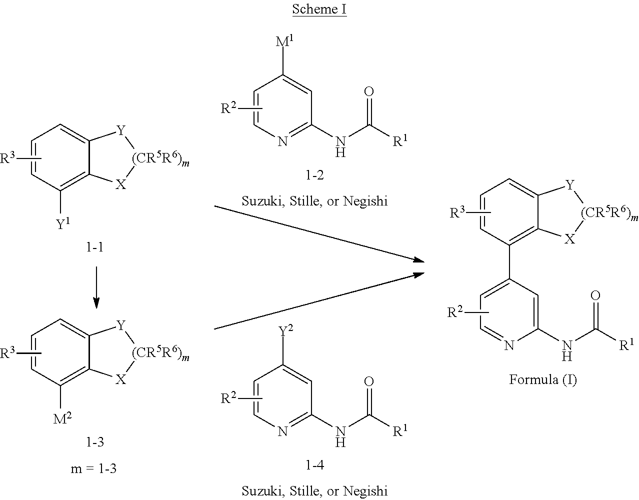

Compounds of Formula (I) can be prepared from optionally protected 1-1 where Y¹ is halogen (e.g., Cl, Br, or I) or pseudohalogen (e.g., OTf or OMs) as shown in Scheme I. 1-1 can be coupled with 1-2, where M¹ is a boronic acid, boronate ester, potassium trifluoroborate, or an appropriately substituted metal, such as Sn(Bu)₃ or Zn, under standard Suzuki conditions (e.g., in the presence of a palladium catalyst, such as tetrakis(triphenylphosphine)palladium(0) or [1,1′-bis(diphenylphosphino)ferrocene]dichloropalladium (II), complex with dichloromethane and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compounds of Formula (I) Alternatively, 1-1 can be converted to an appropriate 1-3 (e.g., M² is B(OH)₂, Bpin, BF₃K, Sn(Bu)₃, or Zn) and then coupled to 1-4 where Y² is halogen (e.g., Cl, Br, or I) or pseudohalogen (e.g., OTf or OMs) under standard Suzuki conditions (e.g., in the presence of a palladium catalyst, such as tetrakis(triphenylphosphine)palladium(0) or [1,1′-bis(diphenylphosphino)ferrocene]dichloropalladium (II), complex with dichloromethane and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium(0) catalyst, such as tetrakis(triphenylphosphine)palladium(0) or [1,1′-bis(diphenylphosphino)ferrocene]dichloropalladium (II)) to give compounds of Formula (I), wherein X, Y, m, R¹, R², R³, R⁵, and R⁶ are as defined herein and above,

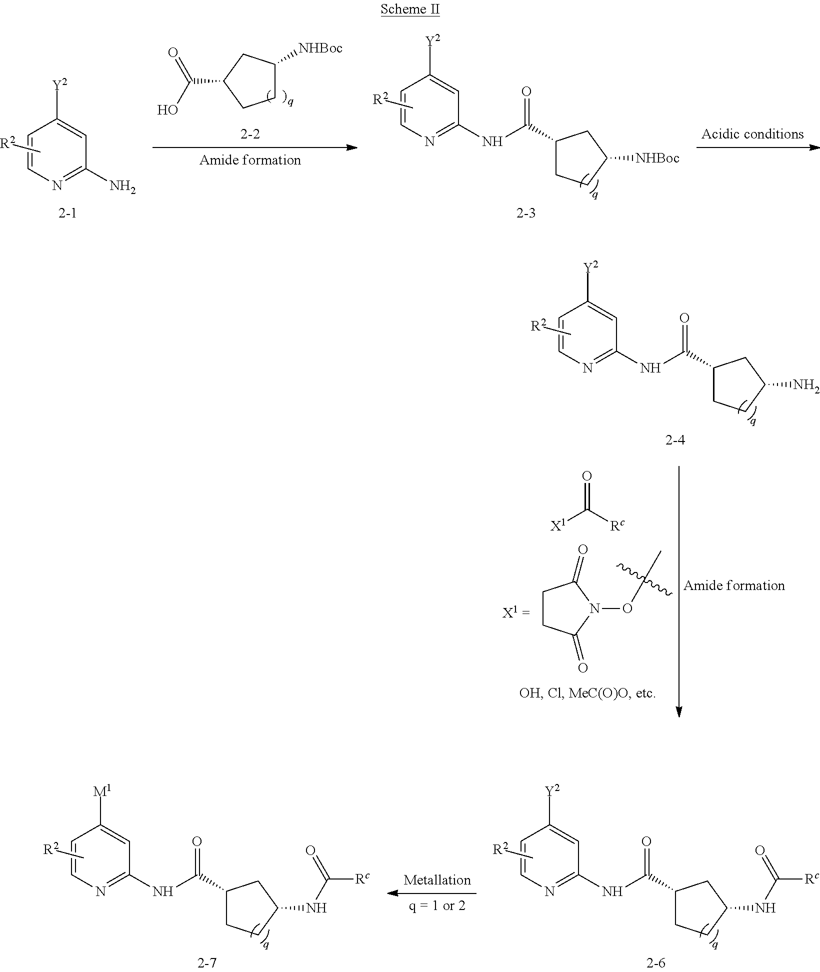

Some intermediates for synthesizing compounds of Formula (I) can be prepared as shown in Scheme II. Optionally substituted 2-aminopyridine 2-1, where Y² is a halogen (e.g., Cl, Br, or I), or pseudohalogen (e.g., OTf or OMs), which can be coupled with Boc-protected amino acid 2-2 as described in US 2016/0376287, which is incorporated herein by its entirety, under standard amide formation conditions (e.g. treatment with an appropriate base, such as DIPEA or trimethylamine and in the presence of coupling agents, such as HATU, HOBt, or PyBOP). The Boc group on compounds 2-3 can be removed under acidic conditions. Amine 2-4 can be coupled to compounds 2-5 under appropriate conditions (e.g., in the presence of a base when 2-5 is a N-hydroxysuccinimide ester, or an acid anhydride, or an acyl chloride; in the presence of coupling agents, such as HATU, HOBt, or PyBOP and a base when 2-5 is a carboxylic acid) to afford amides 2-6. Y² is halo (e.g., Cl, Br, or I) or pseudohalo group (e.g., OTf or OMs) of 2-6, which can be optionally converted to an appropriate substituted metal 2-7 (e.g., M¹ is B(OH)₂, Bpin, BF₃K, Sn(Bu)₃, or Zn) under standard conditions (e.g., in the presence of a diboron reagent, such as bis(pinacolato)diboron, a palladium catalyst, such as dichloro[bis(triphenylphosphoranyl)]palladium or bis(diphenylphosphino)ferrocene]dichloropalladium(II), complex with dichloromethane, and a base, such as potassium acetate). M¹, R², q, and R^{c} are as defined herein and above.

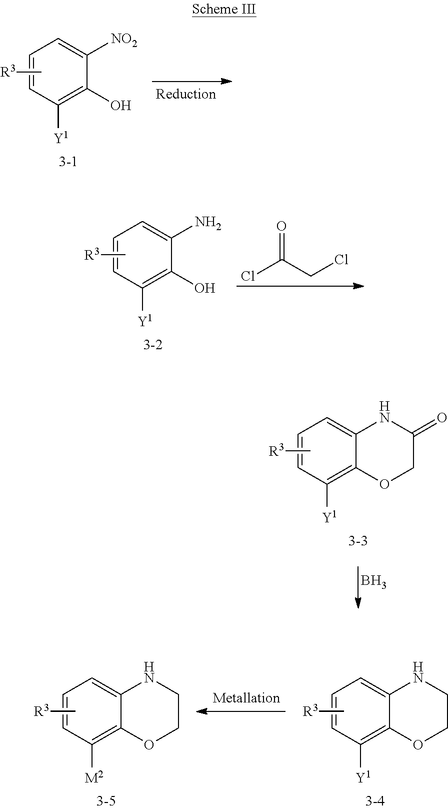

Some intermediates for synthesizing compounds of Formula (V) can be prepared as shown in Scheme III. Nitrophenol 3-1 can be converted to aminophenol 3-2 under reductive conditions such as, but not limited to, Fe/NH₄Cl in MeOH/H₂O or sodium dithionite in EtOH/H₂O. Compounds can react with chloroacetyl chloride in the presence of a base such as DIPEA to give cyclized product 3-3. The carbonyl group on 3-3 can be reduced with borane to afford compounds 3-4. Y¹ halo (e.g., Cl, Br, or I) or pseudohalo group (e.g., OTf or OMs) of 3-4, which can be optionally converted to an appropriate substituted metal 3-5 (e.g., M² is B(OH)₂, Bpin, BF₃K, Sn(Bu)₃, or Zn) under standard conditions (e.g., in the presence of a diboron reagent, such as bis(pinacolato)diboron, a palladium catalyst, such as dichloro[bis(triphenylphosphoranyl)]palladium or bis(diphenylphosphino)ferrocene]dichloropalladium(II), complex with dichloromethane, and a base, such as potassium acetate). R³ is as defined as herein and above.

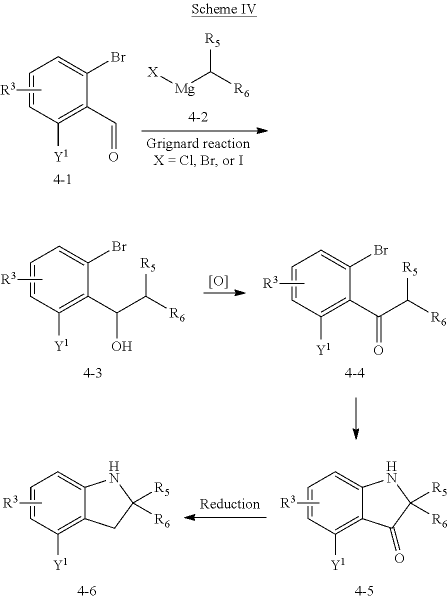

Some intermediates for synthesizing compounds of Formula (XIII-a) can be prepared as shown in Scheme IV. The reaction between aldehyde 4-1 and Grignard reagent 4-2 can afford alcohol 4-3, which can be converted to ketone 4-4 under standard oxidation conditions (e.g., in the presence of PCC, IBX, or DMP). The following Cu catalyzed cyclization could generate compound 4-5. Treatment of compound 4-5 with a reducing reagent such as BH₃ or LAH can y indoline 4-6. Y¹, R³, R⁵, and R⁶ are as defined herein and above.

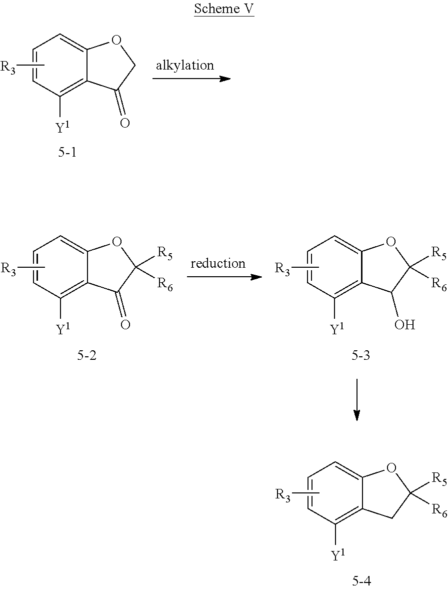

Some intermediates for synthesizing compounds of Formula (XIII-b) can be prepared as shown in Scheme V. Alkylation of compound 5-1 under basic conditions can afford compound 5-2, which can be reduced with NaBH₄ to give alcohol 5-3. The hydroxyl group can be removed upon treatment with a reducing reagent such as Et₃SiH and an acid such as TFA to generate compound 5-4. Y¹, R³, R⁵, and R⁶ are as defined herein and above.

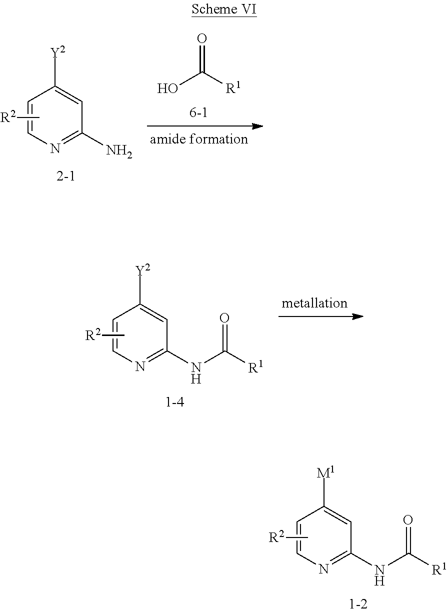

Compounds 1-2 or 1-4 can be prepared as shown in Scheme VI. In the presence of coupling agents, such as HATU, HOBt, or PyBOP and a base such as DIPEA, the coupling between compound 2-1 and acid 6-1 can afford amide 1-4. Y² is halo (e.g., Cl, Br, or I) or pseudohalo group (e.g., OTf or OMs) of amide 1-4, which can be optionally converted to an appropriate substituted metal 1-2 (e.g., M¹ is B(OH)₂, Bpin, BF₃K, Sn(Bu)₃, or Zn) under standard conditions (e.g., in the presence of a diboron reagent, such as bis(pinacolato)diboron, a palladium catalyst, such as dichloro[bis(triphenylphosphoranyl)]palladium or bis(diphenylphosphino)ferrocene]dichloropalladium(II), complex with dichloromethane, and a base, such as potassium acetate). Y², M¹, R², and R¹ are as defined herein and above.

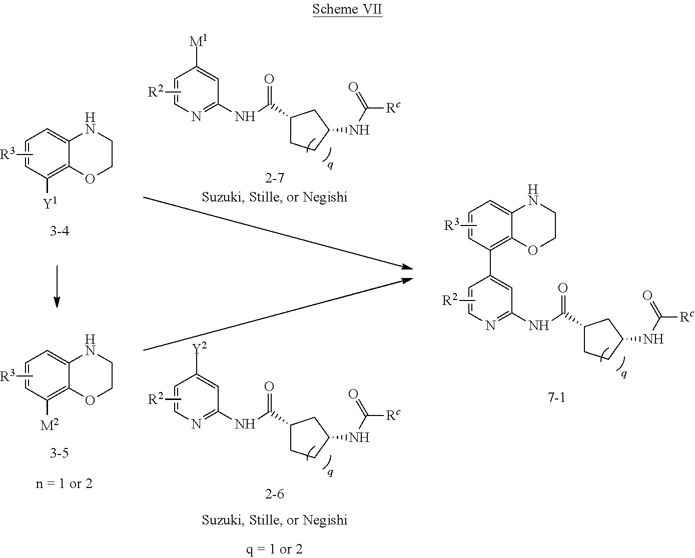

Compounds of 7-1 can be prepared from compounds 3-4 where Y¹ is halogen (e.g., Cl, Br, or I) or pseudohalogen (e.g., OTf or OMs) or 3-5 where M² is B(OH)₂, Bpin, BF₃K, Sn(Bu)₃, or Zn as shown in Scheme VII. 3-4 can be coupled with 2-7, where M¹ is a boronic acid, boronate ester, potassium trifluoroborate, or an appropriately substituted metal, such as Sn(Bu)₃ or Zn, under standard Suzuki conditions (e.g., in the presence of a palladium catalyst, such as tetrakis(triphenylphosphine)palladium(0) or [1,1′-bis(diphenylphosphino)ferrocene]dichloropalladium (II), complex with dichloromethane and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium (II)), to give compound 7-1. Alternatively, coupling between 3-5 (e.g., M² is B(OH)₂, Bpin, BF₃K, Sn(Bu)₃, or Zn) and 2-6 where Y² is halogen (e.g., Cl, Br, or I) or pseudohalogen (e.g., OTf or OMs) under standard Suzuki conditions (e.g., in the presence of a palladium catalyst, such as tetrakis(triphenylphosphine)palladium(0) or [1,1′-bis(diphenylphosphino)ferrocene]dichloropalladium (II), complex with dichloromethane and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium(0) catalyst, such as tetrakis(triphenylphosphine)palladium(0) or [1,1′-bis(diphenylphosphino)ferrocene]dichloropalladium (II)) to give compound 7-1, wherein R², R³, q, and R^{c} are as defined herein and above,

### Example 1: (1S,3R)-3-acetamido-N-(5-chloro-4-(indolin-4-yl)pyridin-2-yl)cyclohexane-1-carboxamide

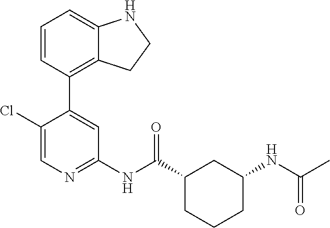 [SMILES: CC(=O)N[C@@H]1CCC[C@H](C(=O)Nc2cc(-c3cccc4c3CCN4)c(Cl)cn2)C1]

### Step 1: tert-butyl N-[(1R,3S)-3-[(4-bromo-5-chloro-2-pyridyl)carbamoyl]cyclohexyl]carbamate

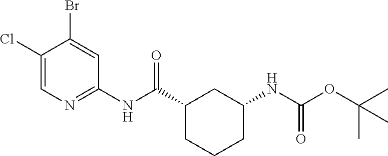 [SMILES: CC(C)(C)OC(=O)N[C@@H]1CCC[C@H](C(=O)Nc2cc(Br)c(Cl)cn2)C1]

To a mixture of (1S,3R)-3-(tert-butoxycarbonylamino)cyclohexanecarboxylic acid [CAS #: 222530-34-9; PharmBlock, batch #PB0000649-119-01] (4.2 g, 17 mmol) in 120 mL of dry DCM at 0° C. was added 1-chloro-N,N,2-trimethylprop-1-en-1-amine (2.77 g, 20.7 mmol)dropwise. The mixture was stirred at room temperature for 1.5 h. Then 4-bromo-5-chloro-pyridin-2-amine (3.58 g, 17.3 mmol) and pyridine (1.68 mL, 20.7 mmol) were added sequentially. The resulted mixture was stirred at room temperature for 12 h. The volatiles were removed under reduced pressure, and the residue was dissolved in 150 mL ethyl acetate, washed with water (50 mL). The organic layer was dried over Na₂SO₄ and concentrated. The residue was purified on reverse phase HPLC (0.1% ammonia in water and methanol) to get tert-butyl N-[(1R,3S)-3-[(4-bromo-5-chloro-2-pyridyl)carbamoyl]cyclohexyl]carbamate (4.8 g, 10.6 mmol, 62% yield) as a white solid. LCMS calcd. for C₁₃H₁₆BrClN₃O₃ [M+2H-tBu]⁺ m/z=376.0; found: 376.0.1H NMR (400 MHz, DMSO-d₆) δ 1.03-1.13 (m, 1H), 1.22-1.32 (m, 3H), 1.37 (s, 9H), 1.68-1.80 (m, 3H), 1.88 (d, J=12.0 Hz, 1H), 2.54-2.61 (m, 1H), 3.21-3.29 (m, 1H), 6.81 (d, J=8.0 Hz, 1H), 8.48 (s, 1H), 8.50 (s, 1H), 10.81 (s, 1H).

### Step 2: (1S,3R)-3-amino-N-(4-bromo-5-chloropyridin-2-yl)cyclohexane-1-carboxamide

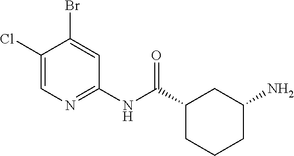 [SMILES: N[C@@H]1CCC[C@H](C(=O)Nc2cc(Br)c(Cl)cn2)C1]

To a mixture of tert-butyl N-[(1R,3S)-3-[(4-bromo-5-chloro-2-pyridyl)carbamoyl]cyclohexyl]carbamate (500 mg, 1.16 mmol) in 10 mL of DCM was added TFA (0.88 mL, 11.55 mmol). The mixture was stirred at room temperature for 5 h. The volatiles were removed under reduced pressure to afford (1S,3R)-3-amino-N-(4-bromo-5-chloropyridin-2-yl)cyclohexane-1-carboxamide as its TFA salt (518 mg, 1.16 mmol, 100% yield). LCMS calcd. for C₁₂H₁₆BrClN₃O [M+H]⁺ m/z=332.01; found: 332.0.

### Step 3: (1S,3R)-3-acetamido-N-(4-bromo-5-chloropyridin-2-yl)cyclohexane-1-carboxamide

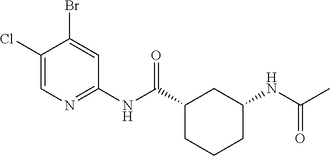 [SMILES: CC(=O)N[C@@H]1CCC[C@H](C(=O)Nc2cc(Br)c(Cl)cn2)C1]

To a mixture of (1S,3R)-3-amino-N-(4-bromo-5-chloro-2-pyridyl)cyclohexanecarboxamide;2,2,2-trifluoroacetic acid (518 mg, 1.16 mmol) in 15 mL of dry DCM at 0° C. was added triethylamine (587 mg, 5.8 mmol), followed by acetic anhydride (142 mg, 1.39 mmol)dropwise. The mixture was stirred at 0° C. for 1 h. The volatiles were removed, and the residue was dissolved in 60 mL of ethyl acetate, washed with water (20 mL). The organic layer was dried over Na₂SO₄ and concentrated. The residue was purified on reverse phase HPLC (0.1% ammonia in water and methanol) to get (1S,3R)-3-acetamido-N-(4-bromo-5-chloro-2-pyridyl)cyclohexanecarboxamide (361 mg, 0.96 mmol, 83% yield) as a white solid. LCMS calcd. for C₁₄H₁₈BrClN₃O₂ [M+H]±m/z=374.02; found: 374.0. ¹H NMR (400 MHz, DMSO-d₆) δ 1.04-1.12 (m, 1H), 1.21-1.32 (m, 3H), 1.71-1.77 (m, 6H), 1.84-1.93 (m, 1H), 2.56-2.63 (m, 1H), 3.51-3.62 (m, 1H), 7.78 (d, J=7.6 Hz, 1H), 8.49 (s, 1H), 8.50 (s, 1H), 10.83 (s, 1H).

### Step 4: [2-[[(1S,3R)-3-acetamidocyclohexanecarbonyl]amino]-5-chloro-4-pyridyl]boronic acid

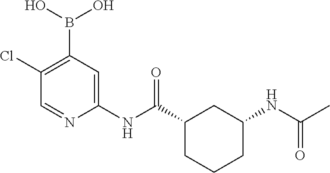 [SMILES: CC(=O)N[C@@H]1CCC[C@H](C(=O)Nc2cc(B(O)O)c(Cl)cn2)C1]

A 20 mL microwave vial with septum containing a mixture of (1S,3R)-3-acetamido-N-(4-bromo-5-chloro-2-pyridyl)cyclohexanecarboxamide (180 mg, 0.48 mmol), bis(pinacolato)diboron (128 mg, 0.50 mmol), [1,1′-bis(diphenylphosphino)ferrocene]dichloropalladium(II), complexed with dichloromethane (39 mg, 0.05 mmol), and potassium acetate (90 mg, 0.92 mmol) was charged with 1,4-dioxane (4.8 mL) and sparged with N₂ for 2 min. The reaction mixture was microwaved at 90° C. for 10 h. LCMS analysis shows complete consumption of starting material, with ˜91% formation of the desired boronic acid. The black mixture was used crude and estimated as a 0.086 M solution of [2-[[(1S,3R)-3-acetamidocyclohexanecarbonyl]amino]-5-chloro-4-pyridyl]boronic acid (5 mL, 0.43 mmol, 90% yield). LCMS calcd for C₁₄H₂₀BClN₃O₄ (M+H)±m/z: 340.12/342.12; found: 340.2/342.2.

### Step 5: (1S,3R)-3-acetamido-N-(5-chloro-4-(indolin-4-yl)pyridin-2-yl)cyclohexane-1-carboxamide

A solution of [2-[[(1S,3R)-3-acetamidocyclohexanecarbonyl]amino]-5-chloro-4-pyridyl]boronic acid (0.25 mL, 0.02 mmol),4-bromoindoline (4.9 mg, 0.02 mmol), [1,1′-Bis(diphenylphosphino)ferrocene]dichloropalladium(II), complex with dichloromethane (1.84 mg, 0 mmol), potassium phosphate tribasic (14.33 mg, 0.07 mmol) in 1,4-dioxane (0.60 mL)/water (0.20 mL) was stirred at 100° C. for 2 h under N₂. LCMS showed almost fully conversion. The reaction was diluted with MeOH, filtered then purified by auto-pure prep-HPLC to give an white solid which is (1S,3R)-3-acetamido-N-[5-chloro-4-(2,3-dihydro-1H-indol-4-yl)pyridin-2-yl]cyclohexane-1-carboxamide (8 mg, 56% yield) as its TFA salt. LCMS m/z calcd for C₂₂H₂₆ClN₄O₂ (M+H)±: 413.2; found: 413.1.

### Example 2: (1S,3R)-3-acetamido-N-(5-chloro-4-(2-methylindolin-4-yl)pyridin-2-yl)cyclohexane-1-carboxamide

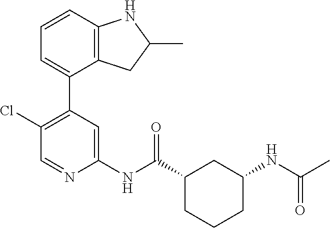 [SMILES: CC(=O)N[C@@H]1CCC[C@H](C(=O)Nc2cc(-c3cccc4c3CC(C)N4)c(Cl)cn2)C1]

The title compound was prepared using a procedure analogous to those described for Example 1, Step 5, with 4-bromo-2-methylindoline replacing 4-bromoindoline. LCMS calcd. for C₂₃H₂₈ClN₄O₂ (M+H)+m/z=427.2; found: 427.1.

### Example 3: (1S,3R)-3-acetamido-N-(5-chloro-4-(1,2,3,4-tetrahydroquinolin-5-yl)pyridin-2-yl)cyclohexane-1-carboxamide

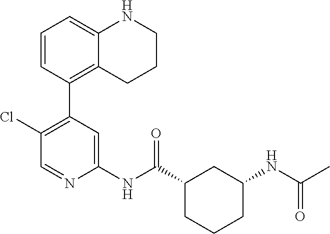 [SMILES: CC(=O)N[C@@H]1CCC[C@H](C(=O)Nc2cc(-c3cccc4c3CCCN4)c(Cl)cn2)C1]

The title compound was prepared using a procedure analogous to those described for Example 1, Step 5, with 5-bromo-1,2,3,4-tetrahydroquinoline replacing 4-bromoindoline. LCMS calcd. for C₂₃H₂₈ClN₄O₂ (M+H)+m/z=427.2; found: 427.1.

### Example 4: (1S,3R)-3-acetamido-N-(5-chloro-4-(3,4-dihydro-2H-benzo[b][1,4]oxazin-8-yl)pyridin-2-yl)cyclohexane-1-carboxamide

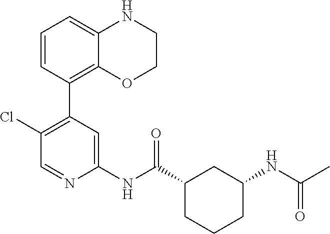 [SMILES: CC(=O)N[C@@H]1CCC[C@H](C(=O)Nc2cc(-c3cccc4c3OCCN4)c(Cl)cn2)C1]

### Step 1: 8-bromo-4H-1,4-benzoxazin-3-one

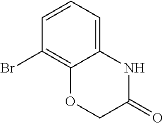 [SMILES: O=C1COc2c(Br)cccc2N1]

To a solution of 2-amino-6-bromophenol (0.8 g, 4.25 mmol) in DME (125 mL), was added 2-chloroacetyl chloride (0.54 mL, 6.81 mmol) and DIPEA (2.11 mL, 12.76 mmol). The mixture was stirred at 85° C. for 6 h. Then K₂CO₃ (2.38 mL, 12.76 mmol) was added, the mixture was stirred anther 6 h at 85° C. The result mixture was concentrated and diluted with water (50 mL), extracted with EA (50 mL). The organic phase was washed with brine, dried over sodium sulfate, concentrated to afford crude 8-bromo-4H-1,4-benzoxazin-3-one (730 mg, 2.72 mmol, 64% yield), which was used in the next step without further purification. LCMS calculated for C₈H₇BrNO₂ (M+H)+m/z=228.0/230.0; found: 228.0/230.0.

### Step 2: 8-bromo-3,4-dihydro-2H-1,4-benzoxazine

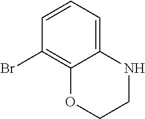 [SMILES: Brc1cccc2c1OCCN2]

To a solution of 8-bromo-2H-1,4-benzoxazin-3(4H)-one (730.0 mg, 3.2 mmol) in THF (10 mL) was added borane-DMS complex (10 M, 0.8 mL, 8 mmol) at 0° C. The mixture was heated to 70° C. for 14 hrs. The resulting mixture was concentrated and diluted with EA, washed with water and brine, dried over sodium sulfate, concentrated and purified by prep-HPLC on a C18 column (20-35 μM, 100 A, 80 g) with mobile phase: H₂O (0.1%© NR₄OH)/MeOH at flow rate: 50 mL/min to give 8-bromo-3,4-dihydro-2H-1,4-benzoxazine (240 mg, 1.12 mmol, 35% yield). LCMS calculated for C₈H₉BrNO (M+H)+m/z=214.0/216.0; found: 214.0/216.0.

### Step 3: (1S,3R)-3-acetamido-N-(5-chloro-4-(3,4-dihydro-2H-benzo[b][1,4]oxazin-8-yl)pyridin-2-yl)cyclohexane-1-carboxamide

The title compound was prepared using a procedure analogous to those described for Example 1, Step 5, with 8-bromo-3,4-dihydro-2H-1,4-benzoxazine replacing 4-bromoindoline. LCMS calcd. for C₂₂H₂₆ClN₄O₃ (M+H)+m/z=429.2; found: 429.1.

### Example 5: (1S,3R)-3-acetamido-N-(5-chloro-4-(2,2-dimethylindolin-4-yl)pyridin-2-yl)cyclohexane-1-carboxamide

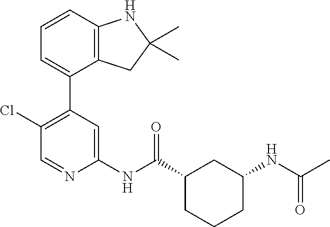 [SMILES: CC(=O)N[C@@H]1CCC[C@H](C(=O)Nc2cc(-c3cccc4c3CC(C)(C)N4)c(Cl)cn2)C1]

### Step 1: 1-(2,6-dibromophenyl)-2-methylpropan-1-ol

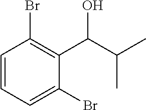

To a solution of 2,6-dibromobenzaldehyde (1.05 g, 4 mmol) in THF (15 mL) was added isopropylmagnesium chloride (2 M in THF, 2.2 mL, 4.4 mmol) dropwise at −10° C. The reaction was warmed up to rt and stirred for 30 min. The reaction was quenched with aqueous NH₄Cl and extracted with EA. The combined organic phase was dried over Na₂SO₄, filtered, and concentrated under a vacuum. The residue was purified by silica column eluting with EA/Hex=1/2 to give 1-(2,6-dibromophenyl)-2-methylpropan-1-ol (460 mg, 37% yield), LCMS calcd for C₁₀H₁₃Br₂O (M+H)⁺ m/z: 307.1/309.1; found: 307.1/309.1.

### Step 2: 1-(2,6-dibromophenyl)-2-methylpropan-1-one

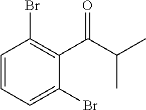

To a stirred solution of 1-(2,6-dibromophenyl)-2-methylpropan-1-ol (460 mg, 1.49 mmol) in DCM (30 mL) was added pyridinium chlorochromate (1.6 g, 7.47 mmol) in one portion. The resulting mixture was stirred at RT for 4 h. To the reaction mixture were added DCM (5 mL) and Celite (200 mg). The resulting suspension was filtered through a Celite pad. The filtrate was concentrated under a vacuum. The residue was purified by silica column eluting with 50% DCM/Hex to give 1-(2,6-dibromophenyl)-2-methylpropan-1-one (440 mg, 96% yield). LCMS calcd. for C₁₀H₁₁Br₂O (M+H)⁺ m/z: 305.1/307.1; found: 305.1/307.1.

### Step 3: 4-bromo-2,2-dimethylindolin-3-one

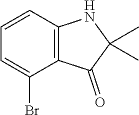

To a solution of 1-(2,6-dibromophenyl)-2-methylpropan-1-one (130.0 mg, 0.42 mmol) in DMF (3 mL) was added L-proline (9.8 mg, 0.08 mmol), potassium carbonate (88 mg, 0.64 mmol), copper sulfate pentahydrate (18.5 mg, 0.08 mmol), L-ascorbic acid sodium salt (19 mg, 0.08 mmol), and sodium azide (33 mg, 0.51 mmol). The mixture was stirred at 70° C. for 16 h. After being cooled to RT, the reaction was diluted with water and MeOH, filtered through a syringe filter. The filtrate was purified by prep-HPLC to give 4-bromo-2,2-dimethyl-1H-indol-3-one (11 mg, 11% yield) as a free base after neutralization. LCMS calcd. for C₁₀H₁₁BrNO (M+H)⁺ m/z: 240.1/242.1; found: 240.1/242.1.

### Step 4: 4-bromo-2,2-dimethylindoline

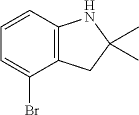

A 4 mL vial was charge with 4-bromo-2,2-dimethyl-1H-indol-3-one (20.0 mg, 0.08 mmol), triethylsilane (0.5 mL, 3.13 mmol), TFA (0.5 mL, 6.53 mmol). The reaction mixture was stirred at 65° C. for 20 h. The volatiles were removed under a vacuum. The residue was dissolved in MeOH 5 mL and purified by prep-HPLC. The fractions were collected, concentrated, neutralized with aqueous NaHCO₃ and extracted with DCM. The organic phases were combined, dried over Na₂SO₄, filtered, and concentrated to give 4-bromo-2,2-dimethyl-1,3-dihydroindole (12 mg, 0.053 mmol, 64% yield). LCMS calcd. for C₁₀H₁₃BrN (M+H)⁺ m/z: 226.0/228.0; found: 226.1/228.1.

### Step 5: (1S,3R)-3-acetamido-N-(5-chloro-4-(2,2-dimethylindolin-4-yl)pyridin-2-yl)cyclohexane-1-carboxamide

A solution of [2-[[(1S,3R)-3-acetamidocyclohexanecarbonyl]amino]-5-chloro-4-pyridyl]boronic acid (Example 1, step 4, 0.25 mL, 0.02 mmol),4-bromo-2,2-dimethyl-1,3-dihydroindole (5.6 mg, 0.02 mmol), [1,1′-Bis(diphenylphosphino)ferrocene]dichloropalladium(II), complex with dichloromethane (1.84 mg), potassium phosphate tribasic (14.33 mg, 0.07 mmol) in 1,4-Dioxane (0.90 mL) and water (0.30 mL) was stirred at 100° C. for 2 h under N₂. The reaction was diluted with MeOH, filtered then purified by prep-HPLC to give (1S,3R)-3-acetamido-N-[5-chloro-4-(2,2-dimethyl-1,3-dihydroindol-4-yl)pyridin-2-yl]cyclohexane-1-carboxamide as its TFA salt (9 mg, 59% yield). LCMS calcd. for C₂₄H₃₀ClN₄O₂ (M+H)⁺ m/z: 441.2; found: 441.0.

### Example 6: (1S,3R)-3-(2-cyanoacetamido)-N-(4-(2,2-dimethylindolin-4-yl)-5-methylpyridin-2-yl)cyclohexane-1-carboxamide

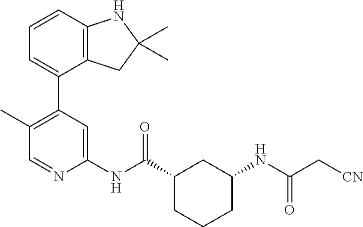 [SMILES: Cc1cnc(NC(=O)[C@H]2CCC[C@@H](NC(=O)CC#N)C2)cc1-c1cccc2c1CC(C)(C)N2]

Step 1: tert-butyl ((1R,3S)-3-((4-iodo-5-methylpyridin-2-yl)carbamoyl)cyclohexyl)carbamate

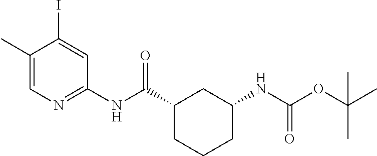 [SMILES: Cc1cnc(NC(=O)[C@H]2CCC[C@@H](NC(=O)OC(C)(C)C)C2)cc1I]

To a mixture of (1S,3R)-3-(tert-butoxycarbonylamino)cyclohexanecarboxylic acid (260 mg, 1.07 mmol, 1.0 eq) in DCM (20 mL) was added 1-chloro-N,N,2-trimethylprop-1-en-1-amine (171 mg, 1.28 mmol, 1.2 eq) at 0° C. The mixture was stirred at room temperature for 1.5 h. Then 4-iodo-5-methyl-pyridin-2-amine (250 mg, 1.07 mmol, 1.0 eq) and pyridine (101 mg, 1.28 mmol, 1.2 eq) were added. The mixture was stirred at room temperature for 12 h. Then the reaction mixture was concentrated and purified by prep-HPLC on a C18 column (20-35 μM, 100 A, 80 g) with mobile phase: H₂O (0.1%© TFA)/MeOH at flow rate: 50 mL/min to give tert-butyl ((1R,3S)-34(4-iodo-5-methylpyridin-2-yl)carbamoyl)cyclohexyl)carbamate (230 mg, 47% yield). LCMS calcd. for C₁₆H₂₇IN₃O₃ (M+H)⁺ m/z=460.1; found: 460.0.

### Step 2: (1S,3R)-3-amino-N-(4-iodo-5-methylpyridin-2-yl)cyclohexane-1-carboxamide

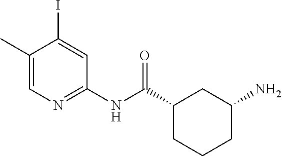 [SMILES: Cc1cnc(NC(=O)[C@H]2CCC[C@@H](N)C2)cc1I]

To a mixture of tert-butyl ((1R,3S)-3-((4-iodo-5-methylpyridin-2-yl)carbamoyl)cyclohexyl)carbamate (230 mg, 0.500 mmol, 1.0 eq) in DCM (5 mL) was added TFA (5.0 mL). The mixture was stirred at room temperature for 5 h. The mixture was concentrated under reduced pressure and dried in vacuum to afford (1S,3R)-3-amino-N-(4-iodo-5-methylpyridin-2-yl)cyclohexane-1-carboxamide as its TFA salt (237 mg, 0.500 mmol, 100% yield). LCMS calcd. for C₁₃H₁₉IN₃O (M+H)⁺ m/z=360.1; found: 360.0.

### Step 3: (1S,3R)-3-(2-cyanoacetamido)-N-(4-iodo-5-methylpyridin-2-yl)cyclohexane-1-carboxamide

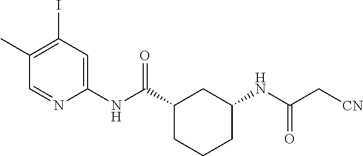 [SMILES: Cc1cnc(NC(=O)[C@H]2CCC[C@@H](NC(=O)CC#N)C2)cc1I]

A 300 mL round-bottomed flask containing a solution of (1R,3S)-3-amino-N-(4-iodo-5-methylpyridin-2-yl)cyclohexane-1-carboxamide 2,2,2-trifluoroacetic acid (1.03 g, 2.18 mmol) in DMF (11 mL) was charged with triethylamine (2.12 mL, 15.24 mmol) followed by cyanoacetic acid N-hydroxysuccinimide ester (0.79 g, 4.35 mmol). The mixture was stirred at room temperature for 2 h, diluted with water and EtOAc. The organic layer was separated, and the aqueous layer was extracted with EtOAc (2×15 mL). The organic layers were combined, washed with water (10 mL)/brine (10 mL), dried over MgSO₄, filtered and concentrated to give (1S,3R)-3-(2-cyanoacetamido)-N-(4-iodo-5-methylpyridin-2-yl)cyclohexane-1-carboxamide (0.78 g, 85% yield). LCMS calcd. for C₁₆H₂₀₁N₄O₂ (M+H)⁺ m/z=427.1; found: 427.0.

### Step 4: (2-0S,3R)-3-(2-cyanoacetamido)cyclohexane-1-carboxamido)-5-methylpyridin-4-yl)boronic acid

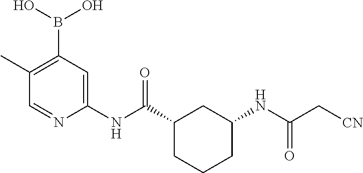 [SMILES: Cc1cnc(NC(=O)[C@H]2CCC[C@@H](NC(=O)CC#N)C2)cc1B(O)O]

A 5 mL microwave vial with septum containing a mixture of (1S,3R)-3-[(2-cyanoacetyl)amino]-N-(4-iodo-5-methylpyridin-2-yl)cyclohexane-1-carboxamide (60.0 mg, 0.14 mmol), bis(pinacolato)diboron (46.5 mg, 0.18 mmol), palladium (II) acetate (3.16 mg, 0.01 mmol), and tricyclohexylphosphane (7.89 mg, 0.03 mmol) and potassium acetate (41.4 mg, 0.42 mmol) was charged with DMSO (2 mL) and sparged with N₂ for 2 min. The reaction mixture heated at 90° C. in a microwave reactor for 12 h. The black mixture was used as a crude solution in the next step, estimated as a 0.07M solution. LCMS calcd. for C₁₆H₂₂BN₄O₄ (M+H)⁺ m/z=345.2; found: 345.1.

### Step 5: (1S,3R)-3-(2-cyanoacetamido)-N-(4-(2,2-dimethylindolin-4-yl)-5-methylpyridin-2-yl)cyclohexane-1-carboxamide

A solution of [2-[[(1S,3R)-3-[(2-cyanoacetyl)amino]cyclohexanecarbonyl]amino]-5-methylpyridin-4-yl]boronic acid (0.2 mL, 0.01 mmol),4-bromo-2,2-dimethyl-1,3-dihydroindole (Example 5, step 4, 3.0 mg, 0.01 mmol), [1,1′-Bis(diphenylphosphino)ferrocene]dichloropalladium(II), complex with dichloromethane (1.14 mg, 0.001 mmol), potassium phosphate tribasic (8.9 mg, 0.04 mmol) in 1,4-dioxane (0.90 mL)/water (0.30 mL) was stirred at 100° C. for 2 h under N₂. The reaction was diluted with MeOH, filtered then purified by prep-HPLC to give an white solid which is (1S,3R)-3-[(2-cyanoacetyl)amino]-N-[4-(2,2-dimethyl-1,3-dihydroindol-4-yl)-5-methylpyridin-2-yl]cyclohexane-1-carboxamide as its TFA salt (4.5 mg, 48% yield). LCMS calcd. for C₂₆H₃₂N₅O₂ (M+H)⁺ m/z=446.2; found: 446.1.

### Example 7: (1S,3R)-3-(2-cyanoacetamido)-N-(4-(3,3-dimethyl-3,4-dihydro-2H-benzo[b][1,4]oxazin-8-yl)-5-methylpyridin-2-yl)cyclohexane-1-carboxamide

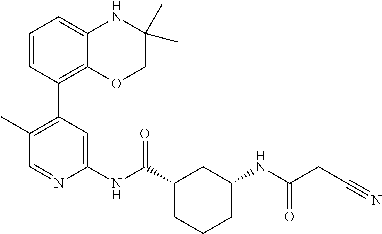 [SMILES: Cc1cnc(NC(=O)[C@H]2CCC[C@@H](NC(=O)CC#N)C2)cc1-c1cccc2c1OCC(C)(C)N2]

### Step 1: 3-Bromo-2-((2-methylallyl)oxy)aniline

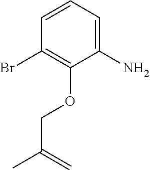

A 100 mL RBF with septum containing a mixture of crude N-(3-bromo-2-hydroxyphenyl)acetamide (1.47 g, 6.38 mmol) in IPA (30 mL) was charged with potassium hydroxide (1.43 g, 25.5 mmol) and purged with N₂. The reaction mixture was stirred at 70° C. for 16 h. The reaction mixture was concentrated under reduced pressure to remove most of the IPA. The residue was diluted with EtOAc, and then washed with water, then brine. The aqueous layers were combined and back-extracted with EtOAc. The organic fractions were combined, dried over Na₂SO₄, filtered and purified by purified by FCC (40 g SiO₂, 0→15% EtOAc in hexanes, wet-loaded in DCM). Fractions containing the product were combined and concentrated under reduced pressure to yield 3-bromo-2-((2-methylallyl)oxy)aniline (750 mg, 3.1 mmol, 49% yield) as a tan/peach solid. LCMS calcd. for C₁₀H₁₃BrNO (M+H)⁺ m/z=242.0/244.0; found: 241.9/243.9.

### Step 2: 1-Azido-3-bromo-2-((2-methylallyl)oxy)benzene

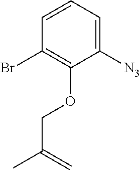

A 100 mL RBF with septum containing 3-bromo-2-(2-methylprop-2-enoxy)aniline (750 mg, 3.1 mmol) under N₂ was sonicated and partially dissolved in 6 N hydrochloric acid (4.0 mL, 24 mmol) and 1.3 mL water. The reaction mixture was cooled to 0° C., and charged with a solution of sodium nitrite (235 mg, 3.41 mmol) in water (2 mL) over 3 min. After 5 additional min, the mixture was slowly charged solid NaHCO₃ until no more gas evolved. The mixture was stirred at 0° C. for 1.5 h. Brown precipitate formed. The reaction mixture was placed at rt and charged with a solution of sodium azide (400 mg, 6.15 mmol) in water (2 mL) over 3 min. The mixture was stirred at rt for 30 min, charged with additional sodium azide (400 mg, 6.15 mmol) in water (2 mL), and stirred at rt for an additional hour. The reaction mixture was diluted with EtOAc (100 mL), washed with water (2×50 mL), and brine (50 mL). The organic layer was dried over Na₂SO₄, filtered, concentrated under reduced pressure to yield 1-azido-3-bromo-2-((2-methylallyl)oxy)benzene as a thick brown/black residue. This crude material was used in the next reaction. LCMS calcd. for C₁₀H₁₁BrNO (M+HN₂)±m/z=240.0/242.0; found: 239.8/241.9.

Step 3: 4-Bromo-1a-methyl-1a,2-dihydro-1H-azirino[1,2-d]benzo[b][1,4]oxazine

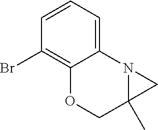

A 100 mL RBF with a Vigreux column, septum, and nitrogen balloon containing a crude solution of 1-azido-3-bromo-2-(2-methylprop-2-enoxy)benzene (831.14 mg, 3.1 mmol) in benzene (40 mL) under N₂ was refluxed in a 100° C. oil bath for 19 h. The reaction solution was concentrated under reduced pressure to yield crude 4-bromo-1a-methyl-1a,2-dihydro-1H-azirino[1,2-d]benzo[b][1,4]oxazine as a brown/black oil and used as is in the following reaction. LCMS calcd. for C₁₀H₁₂BrNO₂ (M+H₂O+H)⁺ m/z=258.0/260.0; found: 257.9/259.8.

### Step 4: 8-Bromo-3,3-dimethyl-3,4-dihydro-2H-benzo[b][1,4]oxazine

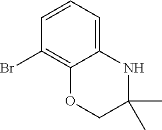

A 100 mL RBF with septum containing a mixture of crude 4-bromo-1a-methyl-1,2-dihydroazirino[2,1-c][1,4]benzoxazine (744 mg theoretical, 3.1 mmol) and platinum(IV) oxide (65 mg, 0.29 mmol) in methanol (60 mL) and ethyl acetate (10 mL) was sparged with N₂ for 5 min, and then was sparged with H₂ for 5 min. The mixture was stirred under a H₂ balloon at rt for 2 h. The crude mixture was sparged with N₂ for 3 min, vacuum filtered through a polypropylene frit, and rinsed with MeOH/EtOAc. The filtrate was concentrated under reduced pressure and purified by FCC (80 g SiO₂, 0→10% EtOAc in hexanes, wet-loaded in DCM). Fractions containing pure major product were concentrated under reduced pressure to yield 8-bromo-3,3-dimethyl-3,4-dihydro-2H-benzo[b][1,4]oxazine (129 mg, 0.53 mmol, 17% yield) as a tan/peach solid. 17% yield over 3 steps from 3-bromo-2-(2-methylprop-2-enoxy)aniline. LCMS calcd. for C₁₀H₁₃BrNO (M+H)⁺ m/z=242.0/244.0; found: 241.9/243.9; 1H NMR (500 MHz, Benzene-d6) δ 7.01 (dd, J=1.4, 8.0 Hz, 1H), 6.50 (t, J=7.9 Hz, 1H), 6.15 (dd, J=1.4, 7.9 Hz, 1H), 3.42 (s, 2H), 2.73 (s, 1H), 0.65 (s, 6H).

### Step 5: 3,3-Dimethyl-8-(4,4,5,5-tetramethyl-1,3,2-dioxaborolan-2-yl)-3,4-dihydro-2H-benzo[b][1,4]oxazine

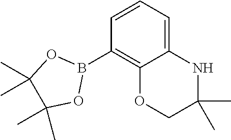

A 20 mL vial containing a mixture of 8-bromo-3,3-dimethyl-3,4-dihydro-2H-benzo[b][1,4]oxazine (126 mg, 0.52 mmol), bis(pinacolato)diboron (143 mg, 0.56 mmol), and potassium acetate (102 mg, 1.0 mmol) was charged with palladium (II) acetate (12 mg, 0.05 mmol), crimped with a septum cap, and evacuated and backfilled with N₂ (3×). The vial was then charged with DMSO (4.6 mL) and sparged with N₂ for 1 min. The reaction mixture was microwaved at 100° C. for 1.5 h. The reaction mixture was charged with dichloro 1,1′-bisdiphenylphosphino)ferrocene palladium (II) dichloromethane (20 mg, 0.02 mmol), and additional bis(pinacolato)diboron (30 mg, 0.12 mmol) and potassium acetate (15 mg, 0.15 mmol), sparged with N₂, and heated at 100° C. for an additional 1 h. The reaction mixture was estimated at ˜90% purity to yield 3,3-dimethyl-8-(4,4,5,5-tetramethyl-1,3,2-dioxaborolan-2-yl)-3,4-dihydro-2H-benzo[b][1,4]oxazine (150 mg, 0.47 mmol, 90% yield), a ˜0.1 M solution in DMSO, and was used as is for the following reaction. LCMS calcd. for C₁₆H₂₅BNO₃ (M+H)⁺ m/z=290.2; found: 290.1.

### Step 6: (1S,3R)-3-(2-Cyanoacetamido)-N-(4-(3,3-dimethyl-3,4-dihydro-2H-benzo[b][1,4]oxazin-8-yl)-5-methylpyridin-2-yl)cyclohexane-1-carboxamide

A 4 mL vial with septum containing sodium carbonate (24.0 mg, 0.23 mmol), [1,1′-bis(diphenylphosphino)ferrocene]dichloropalladium(II), complex with dichloromethane (8.0 mg, 0.01 mmol), and (1S,3R)-3-[(2-cyanoacetyl)amino]-N-(4-iodo-5-methylpyridin-2-yl)cyclohexane-1-carboxamide (Example 6, step 3, 48 mg, 0.11 mmol) under N₂ was charged with a crude reaction mixture of ˜0.1 M 3,3-dimethyl-8-(4,4,5,5-tetramethyl-1,3,2-dioxaborolan-2-yl)-3,4-dihydro-2H-benzo[b][1,4]oxazine (1.15 mL, 0.11 mmol) in DMSO (1.2 mL). The reaction mixture sparged with N₂ for 1 min. The reaction mixture was charged with water (350 μL), sparged with N₂ for an additional minute, and then stirred at 90° C. for 1 h. The reaction mixture was diluted with EtOAc (50 mL), water (25 mL) and sat. NH₄Cl (25 mL), stirred for 15 min at RT, and vacuum filtered through a polypropylene frit. The organic fraction was separated and washed with brine (25 mL). The aqueous fractions were combined and back-extracted with EtOAc (50 mL), and the extract was washed with brine (25 mL). The organic layers were combined and were dried over Na₂SO₄, filtered through cotton, and concentrated under reduced pressure to yield ˜100 mg of crude brown material. The material was purified by prep-LCMS (MeCN in H₂O (w/TFA additive)). Fractions containing desired product were combined and lyophilized to yield (1S,3R)-3-(2-cyanoacetamido)-N-(4-(3,3-dimethyl-3,4-dihydro-2H-benzo[b][1,4]oxazin-8-yl)-5-methylpyridin-2-yl)cyclohexane-1-carboxamide (45 mg, 0.062 mmol, 58% yield) as the TFA salt. LCMS calcd. for C₂₆H₃₂N₅O₃ (M+H)⁺ m/z=462.2; found: 462.1.

### Example 8: (1S,3R)-3-(2-cyanoacetamido)-N-(4-(3,4-dihydro-2H-benzo[b][1,4]oxazin-8-yl)-5-methylpyridin-2-yl)cyclohexane-1-carboxamide

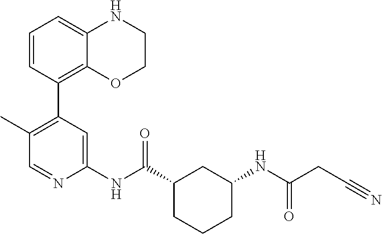 [SMILES: Cc1cnc(NC(=O)[C@H]2CCC[C@@H](NC(=O)CC#N)C2)cc1-c1cccc2c1OCCN2]

### Step 1: 8-(4,4,5,5-Tetramethyl-1,3,2-dioxaborolan-2-yl)-3,4-dihydro-2H-benzo[b][1,4]oxazine

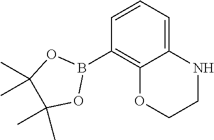 [SMILES: CC1(C)OB(c2cccc3c2OCCN3)OC1(C)C]

A 20 mL vial containing a mixture of 8-bromo-3,4-dihydro-2H-1,4-benzoxazine [CAS #: Vendor] (Example 4, step 2, 52 mg, 0.24 mmol), bis(pinacolato)diboron (66 mg, 0.26 mmol), palladium (II) acetate (5.4 mg, 0.02 mmol), and potassium acetate (47 mg, 0.48 mmol) was evacuated and backfilled with N₂ (3×). The vial was then charged with DMSO (2.1 mL) and sparged with N₂ for 1 min. The reaction mixture was stirred at 100° C. for 1.5 h. The reaction mixture was estimated at ˜90% purity by LCMS to yield 8-(4,4,5,5-tetramethyl-1,3,2-dioxaborolan-2-yl)-3,4-dihydro-2H-1,4-benzoxazine (62.8 mg, 0.22 mmol, 90% yield), estimated as a 0.1 M mixture in DMSO, and used crude/as is for future reactions. LCMS calcd. for C₁₄H₂₁BNO₃ (M+H)⁺ m/z=262.2; found 262.1.

### Step 2: (1S,3R)-3-(2-cyanoacetamido)-N-(4-(3,4-dihydro-2H-benzo[b][1,4]oxazin-8-yl)-5-methylpyridin-2-yl)cyclohexane-1-carboxamide

The title compound was prepared using procedure analogous to those described for Example 7, Step 6, with 8-(4,4,5,5-tetramethyl-1,3,2-dioxaborolan-2-yl)-3,4-dihydro-2H-benzo[b][1,4]oxazine replacing 3,3-dimethyl-8-(4,4,5,5-tetramethyl-1,3,2-dioxaborolan-2-yl)-3,4-dihydro-2H-benzo[b][1,4]oxazine. LCMS calcd. for C₂₄H₂₈N₅O₃ (M+H)⁺ m/z=434.2; found: 434.1.

### Example 9: (1S,3R)-3-acetamido-N-(5-chloro-4-(3,3-dimethyl-3,4-dihydro-2H-benzo[b][1,4]oxazin-8-yl)pyridin-2-yl)cyclohexane-1-carboxamide

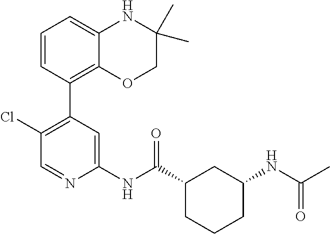 [SMILES: CC(=O)N[C@@H]1CCC[C@H](C(=O)Nc2cc(-c3cccc4c3OCC(C)(C)N4)c(Cl)cn2)C1]

The title compound was prepared using a procedure analogous to those described for Example 1, Step 5, with 8-bromo-3,3-dimethyl-3,4-dihydro-2H-benzo[b][1,4]oxazine (Example 7, step 4) replacing 4-bromoindoline. LCMS calcd. for C₂₄H₃₀ClN₄O₃ (M+H)⁺ m/z=457.2; found: 457.1.

### Example 10: (1S,3R)—N-(5-chloro-4-(3,4-dihydro-2H-benzo[b][1,4]oxazin-8-yl)pyridin-2-yl)-3-(2-cyanoacetamido)cyclohexane-1-carboxamide

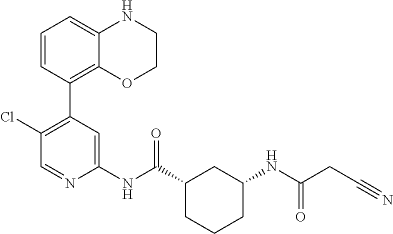 [SMILES: N#CCC(=O)N[C@@H]1CCC[C@H](C(=O)Nc2cc(-c3cccc4c3OCCN4)c(Cl)cn2)C1]

### Step 1: (1S,3R)—N-(4-bromo-5-chloropyridin-2-yl)-3-(2-cyanoacetamido)cyclohexane-1-carboxamide

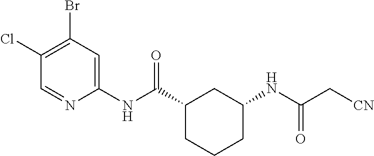 [SMILES: N#CCC(=O)N[C@@H]1CCC[C@H](C(=O)Nc2cc(Br)c(Cl)cn2)C1]

To a stirred solution of (1S,3R)-3-amino-N-(4-bromo-5-chloropyridin-2-yl)cyclohexane-1-carboxamide (Example 1, step 2, 760 mg, 2.28 mmol) in DMF (20 mL) was added triethylamine (0.64 mL, 4.57 mmol) and cyanoacetic acid N-hydroxysuccinimide ester (624 .mg, 3.43 mmol) sequentially. After 30 min, the reaction was cooled to 0° C. and diluted with water (100 mL). The resulting white precipitation was collected and washed with water, dried under vacuum to give (1S,3R)—N-(4-bromo-5-chloropyridin-2-yl)-3-[(2-cyanoacetyl)amino]cyclohexane-1-carboxamide (700 mg, 76% yield).

### Step 2: (1S,3R)—N-(5-chloro-4-(3,4-dihydro-2H-benzo[b][1,4]oxazin-8-yl)pyridin-2-yl)-3-(2-cyanoacetamido)cyclohexane-1-carboxamide

A 4 mL vial with septum containing sodium carbonate (30 mg, 0.28 mmol), [1,1′-bis(diphenylphosphino)ferrocene]dichloropalladium(II), complex with dichloromethane (9.0 mg, 0.01 mmol), and (1S,3R)—N-(4-bromo-5-chloropyridin-2-yl)-3-[(2-cyanoacetyl)amino] cyclohexane-1-carboxamide (50 mg, 0.13 mmol) under N₂ was charged with a crude reaction mixture of −0.1 M 8-(4,4,5,5-tetramethyl-1,3,2-dioxaborolan-2-yl)-3,4-dihydro-2H-1,4-benzoxazine (Example 8, step 1, 1.1 mL, 0.11 mmol) in DMSO (1.2 mL). The reaction mixture sparged with N₂ for 1 min. The reaction mixture was charged with water (350 μL), sparged with N₂ for an additional minute, and then stirred at 90° C. for 1 h. The reaction mixture was diluted with EtOAc (50 mL)/water (25 mL)/sat. aqueous NH₄Cl (25 mL) and filtered. The organic fraction was separated and washed with brine (25 mL). The aqueous fractions were combined and extracted with EtOAc (50 mL), and the extract was washed with brine (25 mL). The organic layers were combined, dried over Na₂SO₄, filtered, and concentrated under reduced pressure to yield ˜80 mg of crude brown material. The material was purified by prep-LCMS (MeCN in H₂O, without TFA additive). Fractions containing desired product were combined and lyophilized to yield (1S,3R)—N-(5-chloro-4-(3,4-dihydro-2H-benzo[b][1,4]oxazin-8-yl)pyridin-2-yl)-3-(2-cyanoacetamido) cyclohexane-1-carboxamide (33 mg, 0.072 mmol, 57% yield). LCMS calcd. for C₂₃H₂₅ClN₅O₃ (M+H)⁺ m/z=454.2/456.2; found: 454.0/456.0.

### Example 11: (1S,3R)—N-(5-chloro-4-(3,3-dimethyl-3,4-dihydro-2H-benzo[b][1,4]oxazin-8-yl) pyridin-2-yl)-3-(2-cyanoacetamido)cyclohexane-1-carboxamide

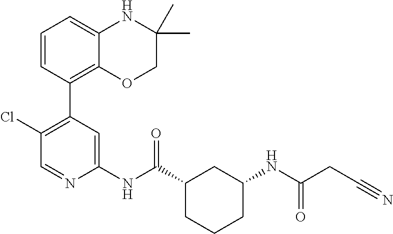 [SMILES: CC1(C)COc2c(cccc2-c2cc(NC(=O)[C@H]3CCC[C@@H](NC(=O)CC#N)C3)ncc2Cl)N1]

The title compound was prepared using procedure analogous to those described for Example 10, Step 2, with 3,3-dimethyl-8-(4,4,5,5-tetramethyl-1,3,2-dioxaborolan-2-yl)-3,4-dihydro-2H-benzo[b][1,4]oxazine (Example 7, step 5) replacing 8-(4,4,5,5-tetramethyl-1,3,2-dioxaborolan-2-yl)-3,4-dihydro-2H-1,4-benzoxazine. LCMS calcd. for C₂₅H₂₉ClN₅O₃ (M+H)⁺ m/z=482.2; found: 482.0.

### Example 12: (1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide

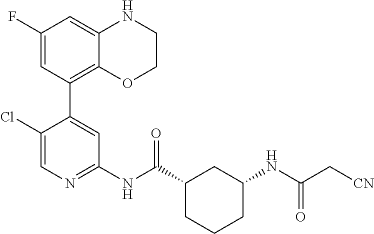 [SMILES: N#CCC(=O)N[C@@H]1CCC[C@H](C(=O)Nc2cc(-c3cc(F)cc4c3OCCN4)c(Cl)cn2)C1]

### Step 1: 2-amino-6-bromo-4-fluorophenol

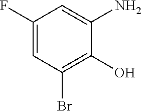

To a solution of 2-bromo-4-fluoro-6-nitrophenol (1.00 g, 4.24 mmol) in ethanol (20 mL) was slowly added a solution of sodium dithionite (4.87 g, 28.0 mmol) in water (20 mL). The reaction mixture was heated at 80° C. for 1 h. Ethanol was removed under reduced pressure and the aqueous phase was extracted with EtOAc (3×50 mL). The combined organic layers were washed with brine, dried over anhydrous Na₂SO₄, and filtered. The filtrate was concentrated to afford 2-amino-6-bromo-4-fluorophenol (670 mg, 3.25 mmol, 77% yield). LCMS calculated for C₆H₆BrFNO (M+H)⁺ m/z=205.9; found: 206.0. ¹H NMR (400 MHz, DMSO-d₆) δ 8.54 (br.s, 1H), 6.48 (dd, J=8.4, 3.2 Hz, 1H), 6.39-6.43 (m, 1H), 5.26 (br.s, 2H).

### Step 2: 8-bromo-6-fluoro-2H-benzo[b][1,4]oxazin-3(4H)-one

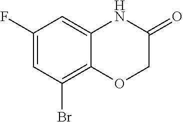 [SMILES: O=C1COc2c(Br)cc(F)cc2N1]

To a solution of 2-amino-6-bromo-4-fluorophenol (670 mg, 3.25 mmol) and chloroacetyl chloride (551 mg, 4.88 mmol) in dimethoxyethane (100 mL) was added N,N-diisopropylethylamine (631 mg, 4.88 mmol). The reaction mixture was heated at 85° C. for 2 h. Then sodium bicarbonate (820 mg, 9.76 mmol) was added into the mixture, and the reaction mixture was heated at 85° C. for another 6 h. The resulted mixture was cooled to room temperature, diluted with water (20 mL), and extracted with EtOAc (3×50 mL). The combined organic layers were washed with brine, dried over anhydrous Na₂SO₄, and filtered. The filtrate was concentrated to afford 8-bromo-6-fluoro-4H-1,4-benzoxazin-3-one (790 mg, 3.21 mmol, 99% yield). 1H NMR (400 MHz, DMSO-d₆) δ 10.96 (s, 1H), 7.15 (dd, J=8.4, 3.2 Hz, 1H), 6.73 (dd, J=9.2, 2.8 Hz, 1H), 4.68 (s, 2H).

### Step 3: 8-bromo-6-fluoro-3,4-dihydro-2H-benzo[b][1,4]oxazine

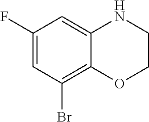 [SMILES: Fc1cc(Br)c2c(c1)NCCO2]

To a solution of 8-bromo-6-fluoro-4H-1,4-benzoxazin-3-one (790 mg, 3.21 mmol) in tetrahydrofuran (80 mL) was added borane-methyl sulfide complex (2 M, 4.01 mL, 8.03 mmol) at 0° C. Then the mixture was heated at 70° C. for 2 d. The mixture was diluted with water (100 mL) and extracted with EtOAc (3×100 mL). The combined organic layers were washed with brine, dried over anhydrous Na₂SO₄, and filtered. The filtrate was concentrated and purified by flash chromatography on a silica gel column (EtOAc in PE 0-20%) to afford 8-bromo-6-fluoro-3,4-dihydro-2H-1,4-benzoxazine (316 mg, 1.36 mmol, 42% yield). LCMS calculated for C₈H₈BrFNO (M+H)⁺ m/z=232.0/234.0; found: 232.0/234.0. ¹H NMR (400 MHz, DMSO-d₆) δ 6.55 (dd, J=8.4, 2.8 Hz, 1H), 6.41 (br.s, 1H), 6.37 (dd, J=10.4, 3.2 Hz, 1H), 4.14-4.16 (m, 2H), 3.30 (t, J=4.0 Hz, 2H).

### Step 4: (5-chloro-2-0S,3R)-3-(2-cyanoacetamido)cyclohexane-1-carboxamido)pyridin-4-yl)boronic acid

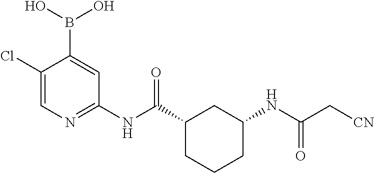 [SMILES: N#CCC(=O)N[C@@H]1CCC[C@H](C(=O)Nc2cc(B(O)O)c(Cl)cn2)C1]

To a mixture of (1S,3R)—N-(4-bromo-5-chloropyridin-2-yl)-3-[(2-cyanoacetyl)amino]cyclohexane-1-carboxamide (Example 10, step 1, 800 mg, 2.00 mmol), and bis(pinacolato)diboron (2.54 g, 10.0 mmol) in toluene (100 mL) was added potassium acetate (647 mg, 6.61 mmol). The reaction mixture was de-gassed under reduced pressure and recharged with N₂ for 3 times and [1,1′-bis(diphenylphosphino)ferrocene]dichloropalladium(II) (146 mg, 0.200 mmol, 0.1 eq) was added. The mixture was heated at 120° C. for 1.5 h. The mixture was concentrated and purified by Prep-HPLC on a C18 column (5 μM, 50×150 mm) with mobile phase: H₂O (0.1% TFA)/MeOH at flow rate: 50 mL/min to give [5-chloro-2-[[(1S,3R)-3-[(2-cyanoacetyl)amino]cyclohexanecarbonyl]amino]pyridin-4-yl]boronic acid (517 mg, 1.42 mmol, 71% yield). LCMS calculated for C₁₅H₁₉BClN₄O₄ (M+H)⁺ m/z=365.2; found: 365.2.

### Step 5: (1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide

A mixture of 8-bromo-6-fluoro-3,4-dihydro-2H-1,4-benzoxazine (100 mg, 0.430 mmol), [5-chloro-2-[[(1S,3R)-3-[(2-cyanoacetyl)amino]cyclohexanecarbonyl]amino]pyridin-4-yl]boronic acid (157 mg, 0.430 mmol) and potassium phosphate (274 mg, 1.29 mmol) in dimethyl sulfoxide (3 mL) and water (0.50 mL) was de-gassed under reduced pressure and recharged with N₂ for 3 times. [1,1′-Bis(diphenylphosphino)ferrocene]dichloropalladium(II) (31.5 mg, 0.040 mmol) was added. The mixture was heated at 93° C. for 4 h. The result mixture was filtered through a Celite pad and the filtrate was purified by flash chromatography on a C18 column (5 μM, 50×150 mm) with mobile phase: H₂O (0.1% formic acid)/MeOH at Flow rate: 50 mL/min to give (1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide (10.2 mg, 0.02 mmol, 5% yield). LCMS calculated for C₂₃H₂₄ClFN₅O₃ (M+H)⁺ m/z=472.2; found: 472.2. ¹H NMR (400 MHz, DMSO-d₆) δ 10.67 (s, 1H), 8.38 (s, 1H), 8.21 (d, J=7.6 Hz, 1H), 8.05 (s, 1H), 6.44 (dd, J=10.4, 2.8 Hz, 1H), 6.34 (s, 1H), 6.17 (dd, J=8.8, 3.2 Hz, 1H), 4.03 (t, J=4.0 Hz, 2H), 3.59 (s, 2H), 3.54-3.58 (m, 1H), 3.29-3.31 (m, 2H), 2.54-2.61 (m, 1H), 1.89-1.92 (m, 1H), 1.77-1.79 (m, 3H), 1.24-1.33 (m, 3H), 1.08-1.10 (m, 1H).

### Example 13: (1S,3R)—N-(5-chloro-4-(6-fluoro-3-methyl-3,4-dihydro-2H-benzo[b][1,4]oxazin-8-yl)pyridin-2-yl)-3-(2-cyanoacetamido)cyclohexane-1-carboxamide

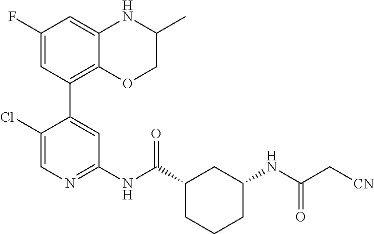 [SMILES: CC1COc2c(cc(F)cc2-c2cc(NC(=O)[C@H]3CCC[C@@H](NC(=O)CC#N)C3)ncc2Cl)N1]

### Step 1: 1-(2-bromo-4-fluoro-6-nitrophenoxy)propan-2-one

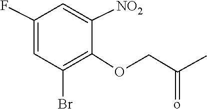

To a stirred solution of 2-bromo-4-fluoro-6-nitrophenol (2.80 g, 11.9 mmol) in acetone (120 mL) were added chloroacetone (1.65 g, 17.8 mmol), sodium carbonate (2.52 g, 23.7 mmol) and sodium iodide (1.78 g, 11.9 mmol). The reaction was stirred at 42° C. for 24 h. The reaction mixture was diluted with water (50 ml), and extracted with EtOAc (3×100 mL). The combined organic layers were washed with brine, dried over anhydrous Na₂SO₄, and filtered. The filtrate was concentrated and the residue was purified on a silica gel column eluting with PE/EA=20/1 to give 1-(2-bromo-4-fluoro-6-nitrophenoxy)propan-2-one (2.60 g, 7.57 mmol, 64% yield). LCMS calculated for C₉H₈BrFNO₄ (M+H)⁺ m/z=291.9; found: 292.0. ¹H NMR (400 MHz, DMSO-d₆) δ 8.11-8.14 (m, 1H), 8.02-8.05 (m, 1H), 4.80 (s, 2H), 2.15 (s, 3H).

### Step 2: 8-bromo-6-fluoro-3-methyl-3,4-dihydro-2H-benzo[b][1,4]oxazine

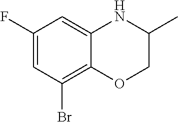

To a solution of 1-(2-bromo-4-fluoro-6-nitrophenoxy)propan-2-one (2.40 g, 8.22 mmol) in ethanol (100 mL) and water (100 mL) were added hydrochloric acid (3.0 mL, 36.0 mmol) and iron (2.29 g, 41.1 mmol) at room temperature. The reaction mixture was heated at 80° C. for 1 h. The reaction mixture was cooled to room temperature, filtered, and washed with EtOH (100 mL). The filtrate was concentrated. The residue was dissolved in dichloromethane (50 mL) and trifluoroacetic acid (50 mL). Then triethylsilane (4.77 g, 41.1 mmol) was added. The resulted mixture was stirred at room temperature for 1 h. The volatiles were removed under reduced pressure and the residue was purified on a silica gel column eluting with ethyl acetate in petroleum 6%-10% to give 8-bromo-6-fluoro-3-methyl-3,4-dihydro-2H-1,4-benzoxazine (1.20 g, 4.87 mmol, 59.3% yield). LCMS calculated for C₉H₁₀BrFNO (M+H)⁺ m/z=245.9; found: 246.0. ¹H NMR (400 MHz, DMSO-d₆) δ 6.55 (dd, J=8.0, 2.8 Hz, 1H), 6.44 (s, 1H), 6.36 (dd, J=10.4, 2.8 Hz, 1H), 4.19-4.22 (m, 1H), 3.64 (dd, J=10.4, 7.6 Hz, 1H), 3.42-3.43 (m, 1H), 1.08 (d, J=6.4 Hz, 3H).

### Step 3: (1S,3R)—N-(5-chloro-4-(6-fluoro-3-methyl-3,4-dihydro-2H-benzo[b][1,4]oxazin-8-yl)pyridin-2-yl)-3-(2-cyanoacetamido)cyclohexane-1-carboxamide

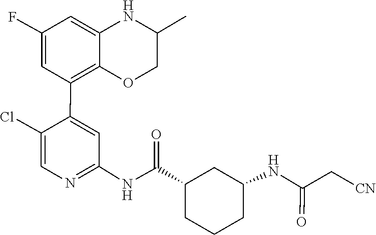 [SMILES: CC1COc2c(cc(F)cc2-c2cc(NC(=O)[C@H]3CCC[C@@H](NC(=O)CC#N)C3)ncc2Cl)N1]

The title compound was prepared using procedure analogous to those described for Example 12, Step 5, with 8-bromo-6-fluoro-3-methyl-3,4-dihydro-2H-benzo[b][1,4]oxazine replacing 8-bromo-6-fluoro-3,4-dihydro-2H-1,4-benzoxazine. LCMS calculated for C₂₄H₂₆ClFN₅O₃ (M+H)⁺ m/z=486.2; found: 486.1. ¹H NMR (400 MHz, CD₃OD) δ 8.28 (s, 1H), 8.04 (s, 1H), 6.42 (dd, J=10.0, 2.8 Hz, 1H), 6.14 (dd, J=8.4, 2.8 Hz, 1H), 4.07 (dd, J=10.4, 2.8 Hz, 1H), 3.71-3.75 (m, 1H), 3.64 (dd, J=10.4, 7.6 Hz, 1H), 3.44-3.50 (m, 1H), 3.30 (s, 2H), 2.56-2.59 (m, 1H), 2.07 (d, J=12.4 Hz, 1H), 1.89-1.95 (m, 3H), 1.44-1.50 (m, 3H), 1.25-1.38 (m, 1H), 1.14 (d, J=6.4 Hz, 3H).

### Example 14: (1S,3R)-3-acetamido-N-(5-chloro-4-(2,2-dimethyl-2,3-dihydrobenzofuran-4-yl)pyridin-2-yl)cyclohexane-1-carboxamide

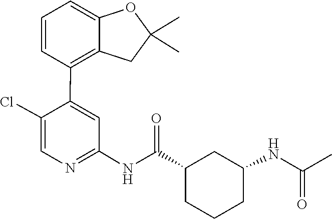 [SMILES: CC(=O)N[C@@H]1CCC[C@H](C(=O)Nc2cc(-c3cccc4c3CC(C)(C)O4)c(Cl)cn2)C1]

### Step 1: 4-bromo-2,2-dimethylbenzofuran-3(2H)-one

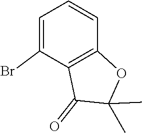

To sodium hydride (60% in mineral oil, 70.4 mg, 1.76 mmol) in THF (5 mL) at −30° C. was added a solution of 4-bromobenzofuran-3(2H)-one (250 mg, 1.17 mmol) in THF (5 mL) dropwise and stirred for 30 mins. Iodomethane (0.22 mL, 3.52 mmol) was added and the resulted mixture was stirred for 1 h at −30° C. and an additional 1 h at room temperature. The reaction was quenched with water, extracted with ethyl acetate, the ethyl acetate layer was washed with water, brine, and dried over sodium sulfate. The organic layer was filtered and concentrated, and the crude was purified by silica gel chromatography eluting with 0-10% EtOAc in hexane to give 4-bromo-2,2-dimethyl-1-benzofuran-3-one (140 mg, 0.58 mmol, 50% yield). LCMS calculated for C₁₀H₁₀BrO₂ (M+H)⁺ m/z=: 241.0; found: 240.9. ¹H NMR (500 MHz, Chloroform-d) δ 7.43 (t, J=8.0 Hz, 1H), 7.21 (dd, J=0.9, 8.0 Hz, 1H), 7.03 (d, J=8.0 Hz, 1H), 1.48 (s, 6H).

### Step 2: 4-bromo-2,2-dimethyl-2,3-dihydrobenzofuran-3-ol

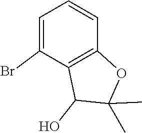

To 4-bromo-2,2-dimethyl-1-benzofuran-3-one (140 mg, 0.58 mmol) in methanol (3 mL) at 0° C. was added sodium borohydride (65.9 mg, 1.74 mmol) and the resulted mixture was stirred at 0° C. for 30 mins. The reaction was quenched with 1 N HCl, and extracted with ethyl acetate. The ethyl acetate layer was washed with water, brine, dried over sodium sulfate, filtered, and concentrated. The crude was purified by silica gel chromatography eluting with 0-15% EtOAc in hexane to give 4-bromo-2,2-dimethyl-3H-1-benzofuran-3-ol (90 mg, 0.37 mmol, 63.7% yield). 1H NMR (500 MHz, Chloroform-d) δ 7.11 (t, J=8.0 Hz, 1H), 7.03 (dd, J=0.9, 8.0 Hz, 1H), 6.74 (d, J=8.0 Hz, 1H), 4.83 (s, 1H), 1.55 (s, 3H), 1.36 (s, 3H).

### Step 3: 4-bromo-2,2-dimethyl-2,3-dihydrobenzofuran

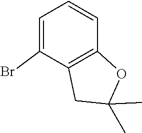

To 4-bromo-2,2-dimethyl-3H-1-benzofuran-3-ol (29.0 mg, 0.12 mmol) in DCM (3 mL) was added the triethylsilane (0.06 mL, 0.36 mmol) and boron trifluoride diethyl etherate (3.39 mg, 0.02 mmol). The reaction was stirred at room temperature for 6 h, quenched with saturated aqueous sodium bicarbonate, and extracted with DCM. The DCM layer was separated, dried over sodium sulfate, filtered, and concentrated. The crude was used in the next step without further purification.

### Step 4: (1S,3R)-3-acetamido-N-(5-chloro-4-(2,2-dimethyl-2,3-dihydrobenzofuran-4-yl)pyridin-2-yl)cyclohexane-1-carboxamide

The title compound was prepared using procedure analogous to those described for Example 1, Step 5, with 4-bromo-2,2-dimethyl-2,3-dihydrobenzofuran replacing 4-bromoindoline. LCMS calcd. for C₂₄H₂₉ClN₃O₃ (M+H)⁺ m/z=442.2; found: 442.2.

### Example 15: (1S,3R)—N-(5-chloro-4-(2,2-dimethyl-2,3-dihydrobenzofuran-7-yl)pyridin-2-yl)-3-(2-cyanoacetamido)cyclohexane-1-carboxamide

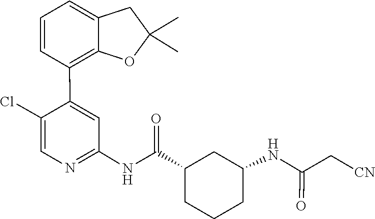 [SMILES: CC1(C)Cc2cccc(-c3cc(NC(=O)[C@H]4CCC[C@@H](NC(=O)CC#N)C4)ncc3Cl)c2O1]

### Step 1: 2,2-dimethyl-2,3-dihydrobenzofuran-7-yl trifluoromethanesulfonate

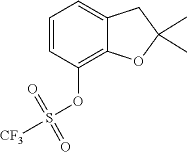 [SMILES: CC1(C)Cc2cccc(OS(=O)(=O)C(F)(F)F)c2O1]

To a solution of 2,2-dimethyl-2,3-dihydrobenzofuran-7-ol (190 μL, 1.27 mmol) and pyridine (206 μL, 2.55 mmol) in DCM (1.3 mL) at 0° C. was added triflic anhydride (287 μL, 1.56 mmol) in DCM (0.50 mL) dropwise. After complete addition, the mixture was warmed to room temperature and stirred for 1.5 hrs. The reaction was then diluted in EtOAc (10 mL) and washed with 1 N HCl (10 ml), NaHCO₃(aq) (10 mL) and brine (10 mL). The organic layer was dried with sodium sulfate and condensed to yield 2,2-dimethyl-2,3-dihydrobenzofuran-7-yl trifluoromethanesulfonate (380 mg, 1.28 mmol, 100% yield) as a yellow oil. 1H NMR (500 MHz, DMSO-d₆) δ 7.28 (d, J=7.4, 1.2 Hz, 1H), 7.21 (d, J=8.3, 1.3 Hz, 1H), 6.91 (t, J=8.4, 7.3 Hz, 1H), 3.13 (s, J=1.1 Hz, 2H), 1.45 (s, 6H). 19F NMR (470 MHz, DMSO-d6) δ −73.44 (3F).

### Step 2: (1S,3R)—N-(5-chloro-4-(2,2-dimethyl-2,3-dihydrobenzofuran-7-yl)pyridin-2-yl)-3-(2-cyanoacetamido)cyclohexane-1-carboxamide

The title compound was prepared using procedure analogous to those described for Example 12, Step 5, with 8-bromo-6-fluoro-3-methyl-3,4-dihydro-2H-benzo[b][1,4]oxazine replacing 8-bromo-6-fluoro-3,4-dihydro-2H-1,4-benzoxazine. LCMS calculated for C₂₅H₂₈ClN₄O₃ (M+H)⁺ m/z=467.2; found: 467.1.

### Biological Assays

CDK9/CyclinT1 Enzymatic Activity Assay

The inhibitory activity of compounds was evaluated in vitro using TR-FRET assay with white 384-well low volume microplate (Greiner Bio-One). CDK9/Cyclin T1 catalyzed phosphorylation of peptides in the presence and absence of compounds was measured and used in IC₅₀ determination. Recombinant protein complex CDK9/Cyclin T1, expressed from insect cell, was purchased from ProQinase. Testing compounds were dissolved in DMSO at 1 mM and tested in 9-dose IC50 mode. The reaction mixture was prepared by mixing CDK9/CyclinT1 (1 nM final), ULight-4E-BP1 (50 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by the dispenser (TECAN D300E) to make a 9.9 μL of the reaction mixture. After 20 minutes of preincubation at room temperature, 0.1 μL MgCl₂ (10 mM final) was added to initiate the reaction. Following a 45 minutes incubation at 37° C., the reaction was stopped by the addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in the dark. The reaction signal was measured by Envision multimode plate reader (PerkinElmer, 2102-0010). IC50 values were determined by fitting the data to the standard 4 parameters with Hill Slope using GraphPad Prism software. See Table B (CDK9 T1).

CDK2/CyclinA2 Enzymatic Activity Assay

The inhibitory activity of compounds was evaluated in vitro using TR-FRET assay with white 384-well low volume microplate (Greiner Bio-One). CDK2/Cyclin A2 catalyzed phosphorylation of peptides in the presence and absence of compounds was measured and used in IC₅₀ determination. Recombinant protein complex CDK2/Cyclin A2, expressed from insect cell, was purchased from ProQinase. Testing compounds were dissolved in DMSO at 1 mM and tested in 9-dose IC50 mode. The reaction mixture was prepared by mixing CDK2/CyclinA2 (1 nM final), ULight-4E-BP1 (50 nM final, Perkinelmer, TRF0128-D), and ATP (1 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by the dispenser (TECAN D300E) to make a 9.9 μL of the reaction mixture. After 20 minutes preincubation at room temperature, 0.1 μL MgCl₂ (10 mM final) was added to initiate the reaction. Following a 45 minutes incubation at 37° C., the reaction was stopped by addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in the dark. The reaction signal was measured by Envision multimode plate reader (PerkinElmer, 2102-0010). IC50 values were determined by fitting the data to the standard 4 parameters with Hill Slope using GraphPad Prism software. See Table B (CDK2 A2).

CDK4/CyclinD1 Enzymatic Activity Assay

The inhibitory activity of compounds was evaluated in vitro using TR-FRET assay with white 384-well low volume microplate (Greiner Bio-One). CDK4/Cyclin D1 catalyzed phosphorylation of peptides in the presence and absence of compounds was measured and used in IC50 determination. Recombinant protein complex CDK4/Cyclin D1, expressed from insect cell, was purchased from ProQinase. Testing compounds were dissolved in DMSO at 1 mM and tested in 9-dose IC50 mode. The reaction mixture was prepared by mixing CDK4/CyclinD1 (1 nM final), ULight-4E-BP1 (100 nM final, Perkinelmer, TRF0128-D), and ATP (2 mM final) in assay buffer (20 mM of HEPES pH 7.4, 1 mM of EGTA, 0.05% BSA, 0.005% Tween 20, and 1 mM TCEP). The compound of interest in DMSO was added to each well in 3-fold serial dilution by the dispenser (TECAN D300E) to make a 9.9 μL of the reaction mixture. After 20 minutes of preincubation at room temperature, 0.1 μL MgCl₂ (10 mM final) was added to initiate the reaction. Following a 45 minutes incubation at 37° C., the reaction was stopped by the addition of 2 μL of quenching buffer consisting of Lance detection buffer (Perkinelmer CR97-100C), LANCE Ultra Europium-anti-P-4E-BP1 (Perkinelmer, TRF0216-D), EDTA, and incubate at room temperature for additional 60 minutes in the dark. The reaction signal was measured by Envision multimode plate reader (PerkinElmer, 2102-0010). IC50 values were determined by fitting the data to the standard 4 parameters with Hill Slope using GraphPad Prism software. See Table B (CDK4 D1).

CellTiter-Glo® Protocol (Proliferation)

Dispense 10 μL aliquot of prepared H929 cells (1:1 ratio of cells: Trypan Blue (#1450013, Bio-Rad)) onto cell counting slide (#145-0011, Bio-Rad) and obtain cell density and cell viability using a cell counter (TC20, Bio-Rad). Remove appropriate volume of resuspended cells from culture flask to accommodate 4000 cells/well at 10 μL/well. Transfer H929 cells to 50 mL conical (#430290, Corning). Spin down at 1000 rpm for 5 min. using a tabletop centrifuge (SPINCHRON 15, Beckman). Discard supernatant and resuspend cell pellet in modified RPMI 1640 (#10-040-CV, Corning) cell culture media containing 10% FBS (F2422-500ML, Sigma), sodium pyruvate (100 mM) (#25-000-CL, Corning), HEPES buffer (1M) (#25-060-CL, Corning) and glucose (200 g/L) (A24940-01, Gibco) to a cell density of 400,000 cells/mL. Dispense 10 μL of resuspended H929 cells per well in 384-well small volume TC treated plate (#784080, Greiner Bio-one) using standard cassette (#50950372, Thermo Scientific) on Multidrop Combi (#5840310, Thermo Scientific) in laminar flow cabinet. Dispense compounds onto plates using a digital liquid dispenser (D300E, Tecan). Incubate plates in humidified tissue culture incubator at 37° C. for 24 hours. Add 10 μL of prepared CellTiTer-Glo® detection buffer (G7570, Promega) to each well of 384-well plate using small tube cassette (#24073295, Thermo Scientific) on Combi multi-drop, incubate at RT for 30-60 min. Read plates with a microplate reader (PheraStar, BMG Labtech) using 384 well luminescence mode. See Table B (Proliferation_CTG_H929).

TABLE B

| | CDK2_ | CDK4_ | CDK9_ | PROLIFERATION_ |
|---|---|---|---|---|
| | A2 | D1 | T1 | CTG_H929 |
| Example | IC₅₀ (nM) | IC₅₀ (nM) | IC₅₀ (nM) | IC₅₀ (nM) |
| 1 | | ++ | ++++ | +++ |
| 2 | | ++ | ++++ | +++ |
| 3 | | + | +++ | +++ |
| 4 | | + | ++++ | +++ |
| 5 | + | +++ | ++++ | +++ |
| 6 | | + | ++++ | |
| 7 | + | + | +++ | ++ |
| 8 | + | + | +++ | ++ |
| 9 | | + | ++++ | |
| 10 | | +++ | ++++ | |
| 11 | | + | ++++ | |
| 12 | ++ | +++ | ++++ | |
| 13 | | +++ | ++++ | |
| 14 | | + | ++++ | ++ |
| 15 | | + | ++++ | |

+: IC₅₀ > 1000 nM;
++: 200 nM < IC₅₀ < 1000 nM;
+++: 20 nM < IC₅₀ < 200 nM;
++++: IC₅₀ < 20 nM

1. A compound, or a pharmaceutically acceptable salt or solvate thereof, having a formula of Formula (I), Formula (II-a), Formula (II-b), Formula (X), or Formula (XI): 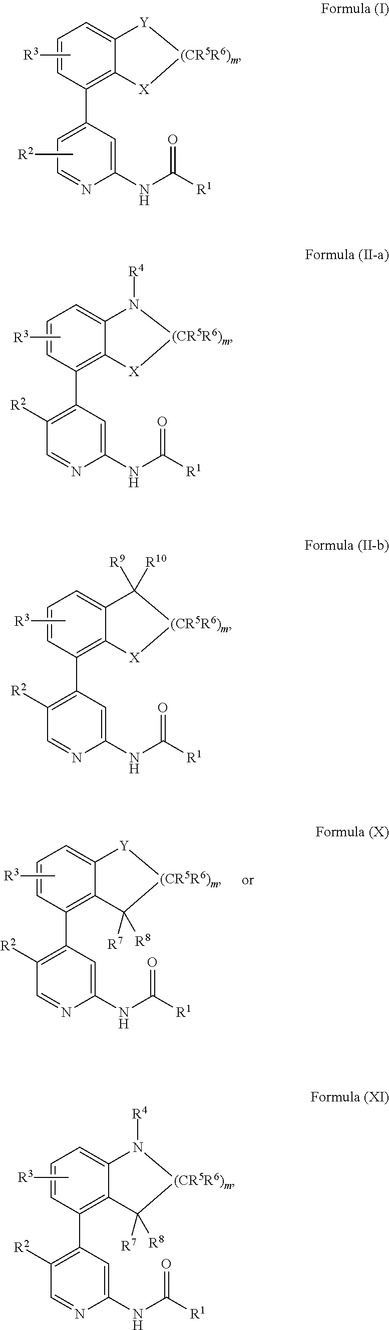
wherein
X is O, S, or CR⁷R⁸;
Y is O, S, CR⁹R¹⁰, or NR⁴;
m is 1-3;
R² and R³ is selected from H, D, halogen, oxo, CN, C₁₋₃ alkyl, C₁₋₃ alkoxy, C₂₋₄ alkenyl, C₂₋₄ alkynyl, C₁₋₃ haloalkyl, and C₁₋₃ haloalkoxy;
R⁴ is selected from H, C₁₋₃ alkyl, C₁₋₃ alkoxy, C₁₋₃ haloalkyl, and —C(O) C₁₋₃ alkyl;
R¹ is selected from C₁₋₆ alkyl, C₁₋₆ alkoxy, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, and (4-14 membered heterocycloalkyl)-C₁₋₄ alkyl;
wherein R¹ is optionally substituted with 1, 2, 3, 4, 5, 6, 7 or 8 independently selected R^{b} substituents;
each R⁵, R⁶, R⁷, R⁸, R⁹, and R¹⁰ are each independently selected from H, D, halo, oxo, C₁₋₆ alkyl, C₁₋₆ alkoxy, C₂₋₆ alkenyl, C₂₋₆ alkynyl, C₁₋₆ haloalkyl, C₁₋₆ haloalkoxy, C₆₋₁₁) aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-14 membered heteroaryl)-C₁₋₄ alkyl, (4-14 membered heterocycloalkyl)-C₁₋₄ alkyl, CN, NO₂, OR^{a1}, SR^{a1}, NHOR^{a1}, C(O)R^{a1}, C(O)NR^{a1}R^{a1}, C(O)OR^{a1}, OC(O)R^{a1}, OC(O)NR^{a1}R^{a1}, NHR^{a1}, NR^{a1}R^{a1}, NR^{a1}C(O)R^{a1}, NR^{a1}C(O)OR^{a1}, NR^{a1}C(O)NR^{a1}R^{a1}, C(═NR^{a1})R^{a1}, C(═NR^{a1})NR^{a1}R^{a1}, NR^{a1}C(═NR^{a1})NR^{a1}R^{a1}, NR^{a1}C(═NOH)NR^{a1}R^{a1}, NR^{a1}C(═NCN)NR^{a1}R^{a1}, NR^{a1}S(O)R^{a1}, NR^{a1}S(O)₂R^{a1}, NR^{a1}S(O)₂NR^{a1}R^{a1}, S(O)R^{a1}, NR^{a1}S(O)(NR^{a1})R^{a1}, S(O)NR^{a1}R^{a1}, S(O)₂R^{a1}, SF₅, P(O)R^{a1}R^{a1}, P(O)(OR^{a1})(OR^{a1}), B(OR^{a1})₂, and S(O)₂NR^{a1}R^{a1};
wherein when R², R³, R⁴, R⁵, R⁶, R⁷, R⁸, R⁹, or R¹⁰ is C₁₋₆ alkyl, C₁₋₆ alkoxy, C₂₋₆ alkenyl, C₂₋₆ alkynyl, C₆₋₁₁) aryl, C₃₋₁₀ cycloalkyl, 5-10 membered heteroaryl, 4-14 membered heterocycloalkyl, C₆₋₁₀ aryl-C₁₋₄ alkyl, C₃₋₁₁) cycloalkyl-C₁₋₄alkyl, (5-14 membered heteroaryl)-C₁₋₄ alkyl, or (4-14 membered heterocycloalkyl)-C₁₋₄ alkyl, then R², R³, R⁴, R⁵, R⁶, R⁷, R⁸, R⁹, and R¹⁰ is optionally substituted with 1, 2, 3, 4 or 5 independently selected R^{b} substituents;
optionally R⁵ and R⁶ together with the carbon atom to which they are both attached form a C₄₋₇ spirocyclic ring optionally substituted with 1, 2, 3, 4 or 5 independently selected R^{b} substituents;
optionally R⁴ and R⁵ together with the atoms to which they are attached form a 4-, 5-, 6-, or 7-membered heterocycloalkyl ring optionally substituted with 1, 2, 3, 4 or 5 independently selected R^{b} substituents;
optionally R⁴ and R⁶ together with the atoms to which they are attached form a 4-, 5-, 6-, or 7-membered heterocycloalkyl ring optionally substituted with 1, 2, 3, 4 or 5 independently selected R^{b} substituents;
optionally R⁷ and R⁸ together with the carbon atom to which they are both attached form a C₃-C₇ spirocyclic ring optionally substituted with 1, 2, 3, 4 or 5 independently selected R^{b} substituents,
optionally one of R⁵ and R⁶ and one of R⁷ and R⁸ together with the atoms to which they are attached form a 4-, 5-, 6-, or 7-membered cycloalkyl ring optionally substituted with 1, 2, 3, 4 or 5 independently selected R^{b} substituents; and
optionally one of R⁵ and R⁶ and one of R⁹ and R¹⁰ together with the atoms to which they are attached form a 4-, 5-, 6-, or 7-membered cycloalkyl ring optionally substituted with 1, 2, 3, 4 or 5 independently selected R^{b} substituents; and
wherein when m is 2 or 3, then two R⁵ or two R⁶ together with the atoms to which they are attached optionally form a 4-, 5-, 6-, or 7-membered cycloalkyl or heterocycloalkyl ring optionally substituted with 1, 2, 3, 4 or 5 independently selected R^{b} substituents;
each R^{a1} is independently selected from H, D, C₁₋₆ alkyl, C₁₋₄haloalkyl, C₂₋₆ alkenyl, C₂₋₆ alkynyl, C₆₋₁₁) aryl, C₃₋₁₀ cycloalkyl, 5-10 membered heteroaryl, 4-14 membered heterocycloalkyl, C₆₋₁₀ aryl-C₁₋₄alkyl, C₃₋₁₁) cycloalkyl-C₁₋₄ alkyl, (5-10 membered heteroaryl)-C₁₋₄ alkyl, and (4-14 membered heterocycloalkyl)-C₁₋₄ alkyl;
wherein when R^{a1} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀cycloalkyl-C₁₋₄ alkyl, (5-10 membered heteroaryl)-C₁₋₄ alkyl- or (4-14 membered heterocycloalkyl)-C₁₋₄ alkyl, then R^{a1} is optionally substituted with 1, 2, 3, 4, or 5 independently selected R^{d} substituents;
each R^{b} substituent is independently selected from D, halo, oxo, C₁₋₄ alkyl, C₁₋₆ alkoxy, C₁₋₄ haloalkyl, C₁₋₄ haloalkoxy, C₆₋₁₀ aryl, C₃₋₁₁) cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, (4-14 membered heterocycloalkyl)-C₁₋₄ alkyl, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NR^{c}C(═NOH)NR^{c}R^{c}, NR^{c}C(═NCN)NR^{c}R^{c}, SF₅, P(O)R^{c}R^{c}, P(O)(OR^{c})(OR^{c}), NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, NR^{c}S(O)(NR^{c})R^{c}, S(O)NR^{c}R^{c}, S(O)₂R^{c}, and S(O)₂NR^{c}R^{c};
wherein when R^{b} is C₁₋₄ alkyl, C₁₋₆ alkoxy, C₁₋₄haloalkyl, C₁₋₄ haloalkoxy, C₆₋₁₀ aryl, C₃₋₁₀ cycloalkyl, 5-10 membered heteroaryl, 4-14 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-14 membered heterocycloalkyl)-C₁₋₄ alkyl, then R^{b} is optionally substituted with 1, 2, or 3 independently selected R^{d} substituents;
each R^{c} is independently selected from H, D, —OH, C₁₋₆ alkyl, C₁₋₆ alkoxy, C₁₋₄ haloalkyl, C₂₋₆alkenyl, C₂₋₆ alkynyl, C₆₋₁₁) aryl, C₃₋₁₁) cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄alkyl, (5-10 membered heteroaryl)-C₁₋₄ alkyl, and (4-10 membered heterocycloalkyl)-C₁₋₄ alkyl;
wherein when R^{c} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{c} is optionally substituted with 1, 2, 3, 4, or 5 independently selected R^{f} substituents;
each R^{f} is independently selected from halogen, C₁₋₄ alkyl, C₁₋₄ haloalkyl, C₂₋₆ alkenyl, C₂₋₆ alkynyl, C₆₋₁₁) aryl, C₃₋₁₀ cycloalkyl, 5-10 membered heteroaryl, 4-10 membered heterocycloalkyl, C₆₋₁₀ aryl-C₁₋₄ alkyl, C₃₋₁₁) cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g} C(═NR^{g})NR^{g}R^{g}, NR^{g} C(═NOH)NR^{g}R^{g}, NR^{g}C(═NCN)NR^{g}R^{g}, SF₅, P(O)R^{g}R^{g}, P(O)(OR^{g})(OR^{g}), S(O)R^{g}, NR^{g}S(O)(NR^{g})R^{g}, S(O)NR^{g}R^{g}, S(O)₂R^{g}, NR^{g}S(O)₂R^{g}, NR^{g} S(O)₂NR^{g}R^{g}, and S(O)₂NR^{g}R^{g};
wherein when R^{f} is C₁₋₄alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, then R^{f} is optionally substituted with 1, 2, 3, 4, or 5 independently selected Rⁿ substituents;
each Rⁿ is independently selected from C₁₋₄ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHOR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, NR^{o}C(═NOH)NR^{o}R^{o}, NR^{o}C(═NCN)NR^{o}R^{o}, SF₅, P(O)R^{o}R^{o}, P(O)(OR^{o})(OR^{o}), S(O)R^{o}, NR^{o}S(O)(NR^{o})R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{d} is independently selected from D, oxo, C₁₋₆ alkyl, C₁₋₆ haloalkyl, halo, C₃₋₁₀ cycloalkyl, C₆₋₁₀ aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, SF₅, P(O)R^{e}R^{e}, P(O)(OR^{e})(OR^{e}), S(O)R^{e}, NR^{e}S(O)(NR^{a1})R^{a1}, S(O)NR^{e}R^{e}, S(O)₂R^{e}, NR^{e}S(O)₂R^{e}, NR^{e}S(O)₂NR^{e}R^{e}, and S(O)₂NR^{e}R^{e},
wherein when R^{d} is C₁₋₆alkyl, C₃₋₁₁) cycloalkyl, C₆₋₁₁) aryl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, or (4-10 membered heterocycloalkyl)-C₁₋₄ alkyl, then R^{d} is optionally substituted with 1, 2, or 3 independently selected R^{f} substituents;
each R^{e} is independently selected from H, D, CN, C₁₋₆ alkyl, C₁₋₄ haloalkyl, C₂₋₆ alkenyl, C₂₋₆ alkynyl, C₆₋₁₁) aryl, C₃₋₁₀ cycloalkyl, 5-10 membered heteroaryl, 4-10 membered heterocycloalkyl, C₆₋₁₀ aryl-C₁₋₄ alkyl, C₃₋₁₁) cycloalkyl-C₁₋₄alkyl, (5-10 membered heteroaryl)-C₁₋₄ alkyl, and (4-10 membered heterocycloalkyl)-C₁₋₄ alkyl,
wherein when R^{e} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{e} is optionally substituted with 1, 2 or 3 independently selected R^{g} substituents;
each R^{g} is independently selected from H, D, C₁₋₆ alkyl, C₁₋₄ haloalkyl, C₂₋₆alkenyl, C₂₋₆ alkynyl, C₆₋₁₁) aryl, C₃₋₁₀ cycloalkyl, 5-10 membered heteroaryl, 4-10 membered heterocycloalkyl, C₆₋₁₀ aryl-C₁₋₄ alkyl, C₃₋₁₁) cycloalkyl-C₁₋₄alkyl, (5-10 membered heteroaryl)-C₁₋₄ alkyl, and (4-10 membered heterocycloalkyl)-C₁₋₄ alkyl,
wherein when R^{g} is C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl- or (4-10 membered heterocycloalkyl)-C₁₋₄ alkyl, then R^{g} is optionally substituted with 1, 2 or 3 independently selected R^{P} substituents;
each R^{P} is independently selected from C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl, C₃₋₁₀ cycloalkyl-C₁₋₄ alkyl, (5-10 membered heteroaryl)-C₁₋₄ alkyl, and (4-10 membered heterocycloalkyl)-C₁₋₄ alkyl, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, SF₅, P(O)R^{r}R^{r}, P(O)(OR^{r})(OR^{r}), S(O)R^{r}, NR^{r}S(O)(NR^{r})R^{r}, S(O)NR^{r}R^{r}, S(O)₂R^{r}, NR^{r}S(O)₂R^{r}, NR^{r}S(O)₂NR^{r}R^{r}, and S(O)₂NR^{r}R^{r};
each R^{o} or R^{r} is independently selected from H, D, C₁₋₄ alkyl, C₃₋₆cycloalkyl, C₆₋₁₀ aryl, 5 or 6-membered heteroaryl, C₁₋₄ haloalkyl, C₂₋₄alkenyl, and C₂₋₄ alkynyl,
wherein when R^{o} or R^{r} is C₁₋₄ alkyl, C₃₋₆ cycloalkyl, C₆₋₁₀ aryl, 5 or 6-membered heteroaryl, C₂₋₄ alkenyl, and C₂₋₄ alkynyl, then R^{o} or R^{r} is optionally substituted with 1, 2 or 3 independently selected R^{q} substituents;
each R^{q} is independently selected from D, OH, CN, —COOH, NH₂, halo, C₁₋₆alkyl, C₁₋₆ haloalkyl, C₁₋₆ alkoxy, C₁₋₆ haloalkoxy, C₁₋₄ alkylthio, phenyl, 5-6 membered heteroaryl, C₃₋₆ cycloalkyl, 4-6 membered heterocycloalkyl, —CONHR¹¹, —NHC(O)R¹¹, —OC(O)R¹¹, C(O)OR¹¹, —C(O)R¹¹, —SO₂R¹¹, —NHSO₂R¹¹, —SO₂NHR¹¹ and NR¹¹R¹¹,
wherein when R^{q} is C₁₋₆ alkyl, phenyl, 4-6 membered heterocycloalkyl or 5-6 membered heteroaryl, then R^{q} is optionally substituted with OH, CN, —COOH, NH₂, C₁₋₆ alkoxy, C₃₋₆cycloalkyl or 4-6 membered heterocycloalkyl; and
each R¹¹ is independently C₁₋₆ alkyl.

2. The compound of claim 1, or a pharmaceutically acceptable salt or solvate thereof, wherein X is O.

3. The compound of claim 2, or a pharmaceutically acceptable salt or solvate thereof, wherein m is 2.

4. The compound of claim 3, or a pharmaceutically acceptable salt or solvate thereof, wherein the compound has a formula of 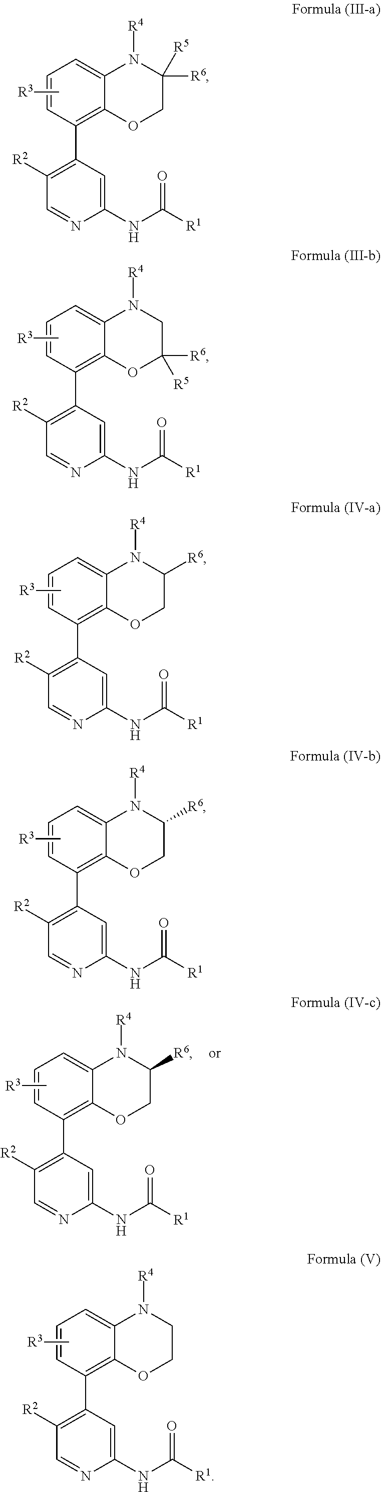 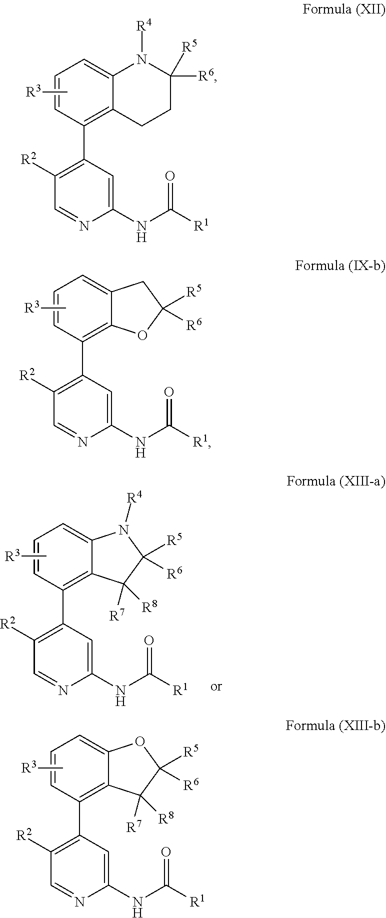
wherein the variables are as defined in claim 1.

5. The compound of claim 4, or a pharmaceutically acceptable salt or solvate thereof, wherein R⁵, R⁶, R⁷ and R⁸ are independently H or C₁₋₆ alkyl.

6. The compound of claim 4, or a pharmaceutically acceptable salt or solvate thereof, wherein both R⁷ and R⁸ are H.

7. The compound of claim 4, or a pharmaceutically acceptable salt or solvate thereof, wherein R⁵ and R⁶ are independently H or C₁₋₆ alkyl.

8. The compound of claim 4, or a pharmaceutically acceptable salt or solvate thereof, wherein both R⁵ and R⁶ are CH₃.

9. The compound of claim 4, or a pharmaceutically acceptable salt or solvate thereof, wherein R⁵ is H.

10. The compound of claim 1, or a pharmaceutically acceptable salt or solvate thereof, wherein R¹ is C₃₋₁₀ cycloalkyl, C₃₋₇ cycloalkyl C₅₋₆ cycloalkyl, each of which is optionally substituted with 1, 2, 3, or 4 independently selected R^{b} substituents.

11. The compound of claim 1, or a pharmaceutically acceptable salt or solvate thereof, wherein R¹ is 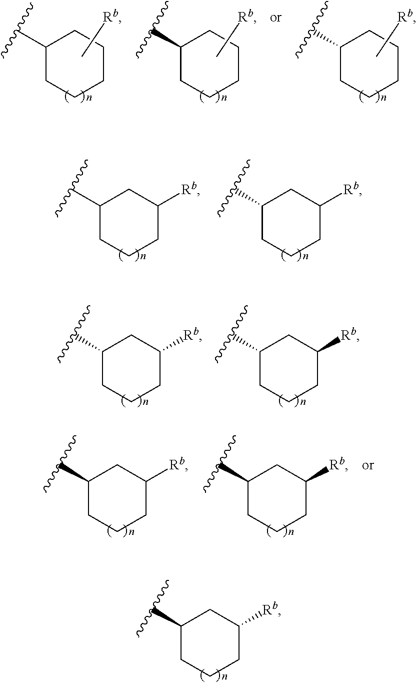
wherein n is 0 or 1 and R^{b} is as defined in claims 1-9.

12. The compound of claim 1, or a pharmaceutically acceptable salt or solvate thereof, wherein R^{b} is NR^{c}C(O)R^{c}, NR^{c}C(O)NR^{c}R^{c}, NRCS(O)(NR^{c})R^{c}, or NRCS(O)₂NR^{c}R^{c}.

13. The compound of claim 12, or a pharmaceutically acceptable salt or solvate thereof, wherein the R^{c} in NR^{c}C(O)R^{c}, NR^{c}C(O)NR^{c}R^{c}, or NR^{c}S(O)₂NR^{c}R^{c} is independently H, C₁₋₆ alkyl, C₃₋₁₀ cycloalkyl, 4-10 membered heterocycloalkyl, or (5-10 membered heteroaryl)-C₁₋₄ alkyl; wherein when R^{c} is C₁₋₆ alkyl, C₁₋₆ alkoxy, C₃₋₁₀ cycloalkyl, 4-10 membered heterocycloalkyl, or (5-10 membered heteroaryl)-C₁₋₄ alkyl, then R^{c} is optionally substituted with 1, 2, 3, 4, or 5 independently selected R^{f} substituents.

14. The compound of claim 13, or a pharmaceutically acceptable salt or solvate thereof, wherein the R^{f} substituents are independently halogen, CN, or OR^{g}.

15. The compound of claim 14, or a pharmaceutically acceptable salt or solvate thereof, wherein the R^{g} is independently H or C₁₋₆ alkyl.

16. The compound of claim 1, or a pharmaceutically acceptable salt or solvate thereof, wherein R^{b} is acetamido (—NHC(O)CH₃), 3-hydroxybutanamido (—NHC(O)CH₂CH(OH)CH₃), propionamido (—NHC(O)CH₂CH₃), 2-methoxyacetamido (—NHC(O)CH₂—OCH₃), 2-cyanoacetamido (—NHC(O)CH₂—CN), 1-hydroxycyclopropane-1-carboxamido, 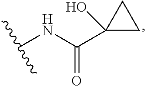 [SMILES: *NC(=O)C1(O)CC1]
2-(thiazol-4-yl)acetamido, 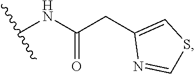 [SMILES: *NC(=O)Cc1cscn1]
methylsulfonamido (—NSO₂CH₃), 3-methylureido (—NC(O)NHCH₃), 3-methoxyureido (—NC(O)NHOCH₃), 3,3-dimethylureido (—NC(O)N(CH₃)₂), or 3-ethylureido (—NC(O)NHCH₂CH₃), morpholine-4-carboxamido, i.e., 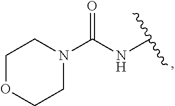 [SMILES: *NC(=O)N1CCOCC1]
or 4-methylpiperazine-1-carboxamide, i.e.,

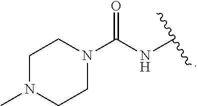 [SMILES: *NC(=O)N1CCN(C)CC1]

17. The compound of claim 16, or a pharmaceutically acceptable salt or solvate thereof, wherein the compound has a formula of 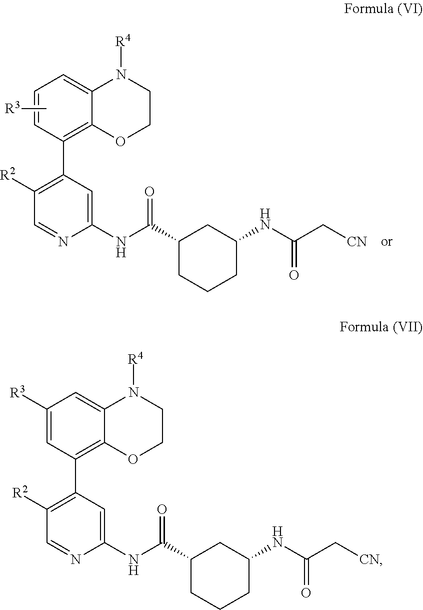
wherein the variables are as defined in claim 1.

18. The compound of claim 1, or a pharmaceutically acceptable salt or solvate thereof, wherein R² is H, halogen, or C₁₋₆ alkyl.

19. The compound of claim 1, or a pharmaceutically acceptable salt or solvate thereof, wherein R² is Cl.

20. The compound of claim 1, or a pharmaceutically acceptable salt or solvate thereof, wherein R¹ is CH₃.

21. The compound of claim 1, or a pharmaceutically acceptable salt or solvate thereof, wherein R³ is H or halogen.

22. The compound of claim 1, or a pharmaceutically acceptable salt or solvate thereof, wherein R³ is F.

23. The compound of claim 1, or a pharmaceutically acceptable salt or solvate thereof, wherein R³ is H

24. The compound of claim 1, or a pharmaceutically acceptable salt or solvate thereof, wherein the compound have a formula of 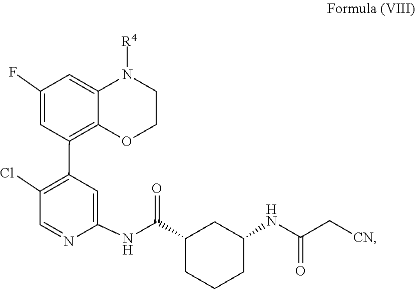
wherein R⁴ is as defined in claim 1.

25. The compound of claim 1, or a pharmaceutically acceptable salt or solvate thereof, wherein R⁴ is H, C₁₋₆ alkyl, or C₁₋₆ alkoxy.

26. The compound of claim 1, or a pharmaceutically acceptable salt or solvate thereof, wherein R⁴ is H.

27. The compound of claim 1, wherein the compound is selected from the group consisting of:
(1S,3R)-3-acetamido-N-(5-chloro-4-(indolin-4-yl)pyridin-2-yl)cyclohexane-1-carboxamide;
(1S,3R)-3-acetamido-N-(5-chloro-4-(2-methylindolin-4-yl)pyridin-2-yl)cyclohexane-1-carboxamide;
(1S,3R)-3-acetamido-N-(5-chloro-4-(1,2,3,4-tetrahydroquinolin-5-yl)pyridin-2-yl)cyclohexane-1-carboxamide;
(1S,3R)-3-acetamido-N-(5-chloro-4-(3,4-dihydro-2H-benzo[b][1,4]oxazin-8-yl)pyridin-2-yl)cyclohexane-1-carboxamide;
(1S,3R)-3-acetamido-N-(5-chloro-4-(2,2-dimethylindolin-4-yl)pyridin-2-yl)cyclohexane-1-carboxamide;
(1S,3R)-3-(2-cyanoacetamido)-N-(4-(2,2-dimethylindolin-4-yl)-5-methylpyridin-2-yl)cyclohexane-1-carboxamide;
(1S,3R)-3-(2-cyanoacetamido)-N-(4-(3,3-dimethyl-3,4-dihydro-2H-benzo[b][1,4]oxazin-8-yl)-5-methylpyridin-2-yl)cyclohexane-1-carboxamide;
(1S,3R)-3-(2-cyanoacetamido)-N-(4-(3,4-dihydro-2H-benzo[b][1,4]oxazin-8-yl)-5-methylpyridin-2-yl)cyclohexane-1-carboxamide;
(1S,3R)-3-acetamido-N-(5-chloro-4-(3,3-dimethyl-3,4-dihydro-2H-benzo[b][1,4]oxazin-8-yl)pyridin-2-yl)cyclohexane-1-carboxamide;
(1S,3R)—N-(5-chloro-4-(3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(3,3-dimethyl-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3,4-dihydro-2H-benzo[b][1,4]oxazin-8-yl)pyridin-2-yl)-3-(2-cyanoacetamido)cyclohexane-1-carboxamide;
(1S,3R)—N-(5-chloro-4-(6-fluoro-3-methyl-3,4-dihydro-2H-benzo[b][1,4]oxazin-8-yl)pyridin-2-yl)-3-(2-cyanoacetamido)cyclohexane-1-carboxamide;
(1S,3R)-3-acetamido-N-(5-chloro-4-(2,2-dimethyl-2,3-dihydrobenzofuran-4-yl)pyridin-2-yl)cyclohexane-1-carboxamide; and
(1S,3R)—N-(5-chloro-4-(2,2-dimethyl-2,3-dihydrobenzofuran-7-yl)pyridin-2-yl)-3-(2-cyanoacetamido)cyclohexane-1-carboxamide.

28. A pharmaceutical composition comprising a compound according to claim 1, or a pharmaceutically acceptable salt or solvate thereof, and optionally a pharmaceutically acceptable excipient.

29. The pharmaceutical composition of claim 28, wherein the pharmaceutical composition comprises an enantiomeric excess of at least 90% of one enantiomer of the compound, or a pharmaceutically acceptable salt or solvate thereof.

30. The pharmaceutical composition of claim 28, wherein the pharmaceutical composition comprises an enantiomeric excess of at least 95% of one enantiomer of the compound, or a pharmaceutically acceptable salt or solvate thereof.

31. The pharmaceutical composition of claim 28, wherein the pharmaceutical composition comprises an enantiomeric excess of at least 98% of one enantiomer of the compound, or a pharmaceutically acceptable salt or solvate thereof.

32. The pharmaceutical composition of claim 28, wherein the pharmaceutical composition comprises an enantiomeric excess of at least 99% of one enantiomer of the compound, or a pharmaceutically acceptable salt or solvate thereof.

33. A method of inhibiting a CDK enzyme comprising: contacting the CDK enzyme with an effective amount of a compound of claim 1, or a pharmaceutically acceptable salt or solvate thereof.

34. The method of claim 33, wherein the CDK enzyme is CDK9.

35. A method of treating a disease or disorder associated with aberrant CDK activity in a subject or a subject in need thereof comprising administering to the subject, a compound of claim 1, or a pharmaceutically acceptable salt or solvate thereof.

36. The method of claim 35, wherein the disease or disorder associated with aberrant CDK activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

37. A method of treating cancer in a subject or a subject in need thereof comprising administering to the subject, a compound of claim 1, or a pharmaceutically acceptable salt or solvate thereof.

38. The method of claim 37, wherein the cancer is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
